(19) United States
(12) Patent Application Publication
(10) Pub. No.: US 2023/0339865 A1
(43) Pub. Date: Oct. 26, 2023

(54) QUINAZOLINE-DERIVED HCK INHIBITORS FOR USE IN THE TREATMENT OF MYD88 MUTATED DISEASES

(71) Applicant: Dana-Farber Cancer Institute, Inc., Boston, MA (US)

(72) Inventors: Nathanael S. Gray, Stanford, CA (US); Steven P. Treon, Los Angele, CA (US); Sara Jean Buhrlage, Somerville, MA (US); Guang Yang, Natick, MA (US); Jinhua Wang, Winchester, MA (US); Li Tan, Pudong, Shanghai (CN); Zachary R. Hunter, Cambridge, MA (US)

(73) Assignee: Dana-Farber Cancer Institute, Inc., Boston, MA (US)

(21) Appl. No.: 18/008,152

(22) PCT Filed: Jun. 3, 2021
(86) PCT No.: PCT/US2021/035677

§ 371 Date: Dec. 2, 2022

Related U.S. Application Data
(60) Provisional application No. 63/035,493, filed on Jun. 5, 2020.

(51) Int. Cl. C07D 239/88; A61K 45/06; A61P 35/00; A61K 31/496; A61K 31/5377; A61K 31/517; C07D 487/04; A61K 39/395; C07D 403/12; C07D 401/12; A61K 31/541

(52) U.S. Cl. CPC C07D 239/88; A61K 45/06; A61P 35/00; A61K 31/496; A61K 31/5377; A61K 31/517; C07D 487/04; A61K 39/39533; C07D 403/12; C07D 401/12; A61K 31/541

(57) ABSTRACT

The present disclosure provides compounds of Formula (I), and pharmaceutically acceptable salts, solvates, hydrates, polymorphs, co-crystals, tautomers, stereoisomers, isotopically labeled derivatives, and prodrugs thereof. The provided compounds may be kinase (e.g., HCK, BTK, LYN) inhibitors. Also provided are pharmaceutical compositions and kits including the provided compounds. Further provided are methods of using the provided compounds, pharmaceutical compositions, and kits (e.g., for treating diseases (e.g., proliferative diseases) in a subject in need thereof). (I)

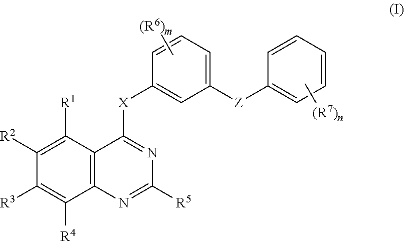

### RELATED APPLICATIONS

The present application claims priority under 35 U.S.C. § 119(e) to U.S. provisional application, U.S. Ser. No. 63/035,493, filed Jun. 5, 2020, which is incorporated herein by reference.

### GOVERNMENT SUPPORT

This invention was made with government support under NIH 2P50CA100707-16A1 (Sub-Project ID: 8568) awarded by the National Institutes of Health. The government has certain rights in the invention.

### BACKGROUND OF THE INVENTION

Hematopoietic cell kinase (HCK) transcription and activation is triggered by mutated myeloid differentiation primary response 88 (MYD88), and is an important determinant of pro-survival signaling. Accordingly, inhibition of the kinase activity of HCK triggers apoptosis in mutated MYD88 cells. For example, the expression of MYD88 mutations in Waldenström's Macroglobulinemia (WM), wherein 95-97% of patients express MYD88^{L265P}, and more rarely non-L265P MYD88 mutations. WM is considered to correspond to lymphoplasmacytic lymphoma (LPL) as defined by the World Health Organization classification system. Up to 30% of patients with Activated B-Cell (ABC) Subtype of Diffuse Large B-cell lymphoma (ABC DLBCL) also express activating MYD88 mutations, including MYD88^{L265P}. Mutations in MYD88 promote Myddosome self-assembly and can trigger NF-kB signaling in the absence of Toll (TLR) or IL1 (IL1R) receptor signaling through IL1 Receptor Associated Kinases (IRAK4/IRAK1) or Bruton's Tyrosine Kinase (BTK).

Next generation sequencing has revealed activating myeloid differentiation primary response 88 (MYD88) mutations in several B-cell malignancies including Waldenström's macroglobulinemia (immunoglobulin M (IgM) secreting lymphoplasmacytic lymphoma), non-IgM secreting lymphoplasmacytic lymphoma, ABC subtype of diffuse large B-cell lymphoma, primary central nervous system (CNS) lymphoma, immune privileged lymphomas that include testicular lymphoma, marginal zone lymphoma, and chronic lymphocytic leukemia. Particularly striking has been the expression of MYD88 mutations in Waldenström's macroglobulinemia (WM), wherein 95-97% of patients express MYD88^{L265P}, and more rarely non-L265P MYD88 mutations. Waldenström's macroglobulinemia is considered to correspond to lymphoplasmacytic lymphoma (LPL) as defined by the World Health Organization classification system. Up to 30% of patients with Activated B-Cell (ABC) Subtype of Diffuse Large B-cell lymphoma (ABC-DLBCL) also express activating MYD88 mutations, including MYD88^{L265P}. Mutations in MYD88 promote Myddosome self-assembly and can trigger NF-kB signaling in the absence of Toll (TLR) or IL1 (IL1R) receptor signaling through IL1 Receptor Associated Kinases (IRAK4/IRAK1) or Bruton's Tyrosine Kinase (BTK).

Ibrutinib is an inhibitor of BTK that is highly active in WM, resulting in responses in 91% of previously treated patients. In WM patients, both major and overall responses to ibrutinib are higher in patients with MYD88 mutations. Ibrutinib also shows activity in previously treated patients with ABC DLBCL, particularly among patients with MYD88 mutations. Ibrutinib is also active in other B-cell malignancies including Chronic Lymphocytic Leukemia (CLL) and Mantle Cell Lymphoma (MCL). Suppression of tonic B-cell receptor (BCR) activity mediated by BTK has been implicated as the mechanism underlying ibrutinib activity in non-WM B-cell diseases.

### SUMMARY OF THE INVENTION

Provided herein are methods of treating a disease a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))) in a subject in need thereof, comprising administering to the subject a therapeutically effective amount of a compound of Formula (I). In certain aspects, provided herein are methods for inhibiting a mutated BTK (e.g., a C481S mutant) in a subject in need thereof.

Also provided herein are compounds of Formula (I):

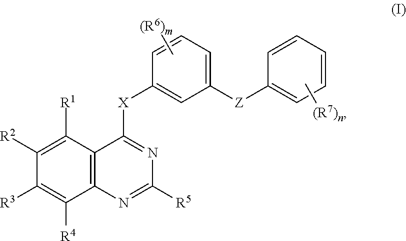

wherein R¹, R², R³, R⁴, R⁵, R⁶, R⁷, X, and Z are as defined herein, and pharmaceutically acceptable salts, solvates, hydrates, polymorphs, co-crystals, tautomers, stereoisomers, isotopically labeled derivatives, and prodrugs thereof.

In certain embodiments, a compound of Formula (I) is of formula:

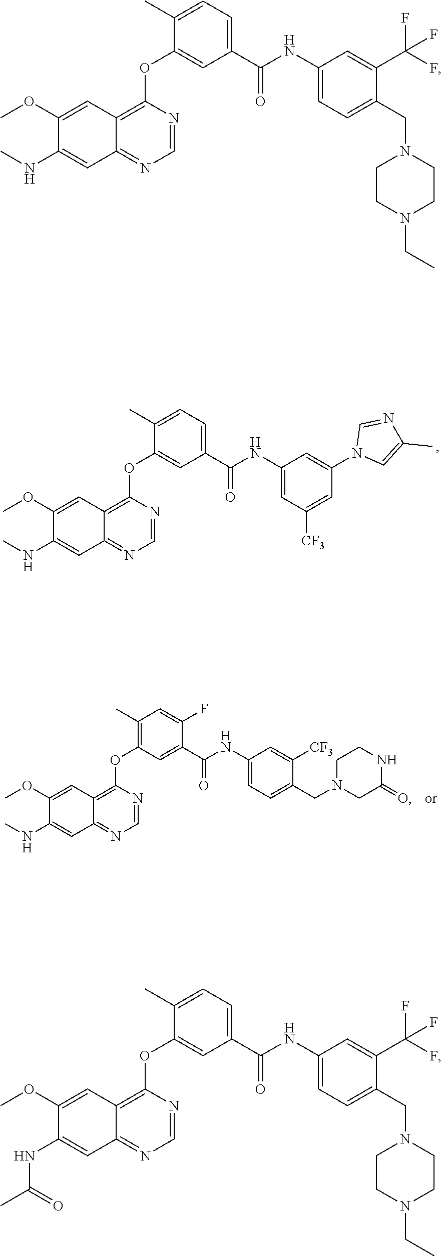

or a pharmaceutically acceptable salt, solvate, hydrate, polymorph, co-crystal, tautomer, stereoisomer, isotopically labeled derivative, or prodrug thereof.

The provided compounds may be kinase (e.g., an SRC family kinase (i.e. SFK) (e.g., HCK, LYN), a Tec family kinase (e.g., BTK)) inhibitors, and in certain aspects, the compounds may be specific or selective for SFKs (e.g., HCK, LYN) or Tec family kinases (e.g., BTK) over one or more other kinases. Also provided are pharmaceutical compositions and kits comprising the disclosed compounds. The present disclosure also provides methods of using the disclosed compounds, pharmaceutical compositions, and kits (e.g., for treating a disease (e.g., a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))) in a subject in need thereof, or inhibiting the activity of a kinase in a subject in need thereof, a biological sample, or a cell).

In yet another aspect, the present disclosure provides compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, for use in the treatment and/or prevention of a disease (e.g., a proliferative disease, such as an IgM gammopathy, mastocytosis, or cancer) in a subject in need thereof.

In another aspect, the present disclosure provides uses of compounds of Formula (I), and pharmaceutically acceptable salts, solvates, hydrates, polymorphs, co-crystals, tautomers, stereoisomers, isotopically labeled derivatives, and prodrugs thereof, and pharmaceutical compositions thereof, in the manufacture of a medicament for treating and/or preventing a disease in a subject in need thereof.

In another aspect, the present disclosure provides methods of preparing a compound of Formula (I), or a pharmaceutically acceptable salt, solvate, hydrate, polymorph, co-crystal, tautomer, stereoisomer, isotopically labeled derivative, or prodrug thereof.

In one aspect, the present disclosure provides pharmaceutical compositions including a compound described herein, and a pharmaceutically acceptable excipient. In certain embodiments, the pharmaceutical composition further comprises an additional pharmaceutical agent. In certain embodiments, the additional pharmaceutical agent is selected from the group consisting of chemotherapy drugs, epigenetic modifiers, glucocorticoids, biologics, and immunotherapy agents. In another aspect, the additional pharmaceutical agents is a BCL-2 inhibitor (e.g., venetoclax, navitoclax, obatoclax).

The pharmaceutical compositions may be useful for treating a disease in a subject in need thereof, inhibiting the activity of a kinase in a subject in need thereof, a biological sample, or a cell. In certain aspects, the disease is a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))).

The present disclosure provides methods of treating a disease in subject by administering to a subject in need thereof an effective amount of a compound, or a pharmaceutical composition thereof, as described herein. In certain aspects, the disease is a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))). Also described are methods for contacting a biological sample or cell with an effective amount of a compound, or pharmaceutical composition thereof, as described herein. In certain embodiments, a method described herein further includes administering to the subject in need thereof an additional pharmaceutical agent. In certain embodiments, a method described herein further includes contacting the biological sample or cell with an additional pharmaceutical agent.

In one aspect, provided are methods for treating a disease in a subject who is resistant to treatment with a BTK inhibitor (e.g., ibrutinib, CC-292, ONO-4059, evobrutinib, spebrutinib, BGB-3111, HM71224, or ACP-196 (i.e., acalabrutinib)). In certain aspects, a subject who is resistant to treatment with a BTK inhibitor has a mutated BTK (e.g., a C481S mutated BTK). In another aspect, the subject who is resistant to treatment with a BTK inhibitor is diagnosed to have an MYD88 mutated disease (e.g., a proliferative disease (e.g., IgM gammopathy, mastocytosis, cancer)).

In another aspect, provided herein are methods of inhibiting a kinase (e.g., SFK (e.g., HCK, LYN), Tec family kinases (e.g., BTK)) in a subject in need thereof, comprising administering to the subject a therapeutically effective amount of a compound of Formula (I), or a pharmaceutically acceptable salt, solvate, hydrate, polymorph, co-crystal, isotopically-labeled derivative, stereoisomer, or prodrug thereof. In certain aspects, provided herein are methods for inhibiting a mutated BTK (e.g., a C481S mutant) in a subject in need thereof.

In certain aspects, the method further comprises administering an anti-cancer agent to the subject. In some embodiments, the anti-cancer agent is a chemotherapeutic agent. In another aspect, the method further comprises administering to the subject one or more of a proteasome inhibitor (e.g., bortezomib, carfilzomib, ixazomib, or oprozomib), a monoclonal antibody (e.g., rituximab, daratumumab, ofatumumab, or obinutuzumab), an alkylator drug (e.g., bendamustine, cyclophosphamide), a nucleoside analogue (e.g., fludarabine or cladribine), an mTOR inhibitor (e.g., everolimus), a BTK inhibitor (e.g., ibrutinib, acalabrutinib, or BGB-3111), a BCR inhibitor (e.g., a SYK inhibitor) and/or an immunomodulating agent (e.g., thalidomide or lenalidomide).

In certain embodiments, the proteasome inhibitor is bortezomib. In certain embodiments, the proteasome inhibitor is carfilzomib. In certain embodiments, the proteasome inhibitor is ixazomib. In certain embodiments, the proteasome inhibitor is oprozomib.

In certain embodiments, the monoclonal antibody is rituximab. In certain embodiments, the monoclonal antibody is daratumumab. In certain embodiments, the monoclonal antibody is ofatumumab. In certain embodiments, the monoclonal antibody is obinutuzumab).

In certain embodiments, the CXCR4 antagonist is KRH-3955. In certain embodiments, the CXCR4 antagonist is plerixafor. In certain embodiments, the CXCR4 antagonist is motixafortide. In certain embodiments, the CXCR4 antagonist is a T140 analog.

In another aspect, the present disclosure provides kits comprising: a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof; and instructions for using the compound, or a pharmaceutically acceptable salt, solvate, hydrate, polymorph, co-crystal, tautomer, stereoisomer, isotopically labeled derivative, or prodrug thereof, or the pharmaceutical composition.

The details of one or more embodiments of the present disclosure are set forth herein. Other features, objects, and advantages of the present disclosure will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” as used herein includes alkyl, alkenyl, alkynyl, cycloalkyl, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in —CH).

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more (e.g., two, three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

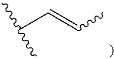

may be in the (E)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₅ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C=triple bonds in the carbocyclic ring is referred to as “cycloalkynyl.” Carbocyclyl includes aryl. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅_cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Partially unsaturated heterocyclyl groups includes heteroaryl. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5.6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6.6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi-electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 pi-electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5.6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6.6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{cc})₂, —P(═O)(OR^{cc})₂, —OP(═O)(R^{cc})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂-6 alkenyl, heteroC₂₋₆alkynyl, C₃₋₁₀ carbocyclyl, C₆₋₁₀ aryl, 3-10 membered heterocyclyl, or 5-10 membered heteroaryl; or two geminal R^{gg} substituents are joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻), B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonoethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneimine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfonamide (Nps), 2,4-dinitrobenzenesulfenamide, pentachlorobenzenesulfenamide, 2-nitro-4-methoxybenzenesulfonamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{a}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

These and other exemplary substituents are described in more detail in the Detailed Description, Examples, Figures, and Claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” refer to compounds that are interchangeable forms of a particular compound structure, and that vary in the displacement of hydrogen atoms and electrons. Thus, two structures may be in equilibrium through the movement of pi-electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I), which have cleavable groups and become by solvolysis or under physiological conditions the compounds of Formula (I) which are pharmaceutically active in vivo. Such examples include, but are not limited to, ester derivatives and the like. Other derivatives of the compounds of this disclosure have activity in both their acid and acid derivative forms, but in the acid sensitive form often offers advantages of solubility, tissue compatibility, or delayed release in the mammalian organism (See, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this disclosure are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₆-C₁₂ substituted aryl, and C₆-C₁₂ arylalkyl esters of the compounds of Formula (I) may be preferred.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. A non-human animal may be a transgenic animal. A subject who is resistant to treatment with a BTK inhibitor is one who shows no or minimal response to the treatment. In some embodiments, response to a treatment is measured by reduction in tumor cells or tumor cell killing. In some embodiments, response to a treatment is measured by changes in symptoms of the disease, condition or malignancy (e.g., a proliferative disease). It has been discovered that the compounds that block ATP binding to HCK as described herein are able to cause tumor cell killing even in cells that are derived from subjects who are resistant to a BTK inhibitor treatment.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration,” refers to implanting, absorbing, ingesting, injecting, inhaling, applying, or otherwise introducing a compound, or a pharmaceutical composition thereof, to a subject in need thereof, a biological sample, or a cell.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, auto inflammatory diseases, and autoimmune diseases.

The terms “neoplasm” and “tumor” are used interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

A “protein” or “peptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, or synthetic, or any combination of these.

The term “kinase” refers to any enzyme that catalyzes the addition of phosphate groups to an amino acid residue of a substrate (e.g., a protein or nucleoside). For example, a serine kinase catalyzes the addition of a phosphate group to serine residue in a protein. In certain embodiments, the kinase is a tyrosine kinase. Examples of kinases include, but are not limited to, a Janus kinase (e.g., SRC family kinases (e.g., HCK, LYN), Tec family kinases (e.g., BTK), Janus kinase 1 (JAK1), Janus kinase 2 (JAK2), Janus kinase 3 (JAK3), tyrosine kinase 2 (TYK2)), a CMGC kinase (e.g., a cyclin-dependent kinase (CDK, e.g., CDK1, CDK2, CDK2, CDK4, CDK5, CDK7, CDK8, CDK9, CDK10, CDK11, CDK12, CDK13, CDK14, CDK16, CDK20), a mitogen-activated protein kinase (MAPK, e.g., MAPK1, MAPK3, MAPK4, MAPK6, MAPK7, MAPK8, MAPK9, MAPK10, MAPK11, MAPK12, MAPK13, MAPK14, MAPK15), a glycogen synthase kinase 3 (GSK3, e.g., GSK3a, GSK3P), or a CDC-like kinase (CLK, e.g., CLK1, CLK2, CLK3, CLK4)), an AGC kinase (e.g., protein kinase A (PKA), protein kinase C (PKC), protein kinase G (PKG)), a Ca²⁺/calmodulin-dependent protein kinase (CaM kinase, e.g., a specialized CaM kinase, a multifunctional CaM kinase), a casein kinase 1 (CK1, e.g., CK1 alpha, CK1 beta 1, CK1 gamma 1, CK1 gamma 2, CK1 gamma 3, CK1 delta, CK1 epsilon), a STE kinase (e.g., a homolog of yeast Sterile 7, Sterile 11, or Sterile 20 kinase), a tyrosine kinase (TK, e.g., a receptor tyrosine kinase (RTK), a non-receptor tyrosine kinase (nRTK)), and a tyrosine-kinase-like kinase (TKL, e.g., a mixed lineage kinase (MLK), RAF, a serine threonine kinase receptor (STKR), a leucine rich repeat kinase (LRRK), a LIM domain kinase (LIMK), a testis expressed serine kinase (TESK), an IL1 receptor associated kinase (IRAK), a receptor interacting protein kinase (RIPK)).

HCK is a member of the src-family of protein tyrosine kinases, and is aberrantly up-regulated in WM cells. In myeloma cells, HCK is activated by interleukin 6 (IL6) through the IL6 co-receptor IL6ST (GP130).

Bruton's tyrosine kinase (BTK) is a member of the src-related BTK/Tec family of cytoplasmic tyrosine kinases, is required for B cell receptor signaling, plays a key role in B-cell maturation, and exhibits increased activation in a number of B-cell malignancies.

LYN proto-oncogene (LYN) is a member of the src-family of protein tyrosine kinases, plays an important role in the regulation of B-cell differentiation, proliferation, survival, and apoptosis, is important for immune self-tolerance, and acts downstream of several immune receptors, including the B-cell receptor (BCR). Without wishing to be bound by any particular theory, BCR signaling is thought to be involved in pro-growth and survival signaling in MYD88 mutated disease, as well as being involved in non-MYD88 mutated disease. For example, BCR signaling is thought to be active in Waldenström's macroglobulinemia, ABC subtype of diffuse large B-cell lymphoma, and chronic lymphocytic leukemia.

Proto-oncogene tyrosine-protein kinase SRC (SRC) is a protein tyrosine kinase, plays a central role in the regulation of a variety of biological processes, such as cell proliferation, migration, adhesion, and survival in solid tumors, and is overexpressed in Waldenström's macroglobulinemia.

As used herein “inhibition”, “inhibiting”, “inhibit,” and “inhibitor”, and the like, refer to the ability of a compound to reduce, slow, halt, block, or prevent activity of a particular biological process (e.g., a kinase (e.g., SFK (e.g., HCK, LYN), Tec family kinases (e.g., BTK)) in a cell relative to vehicle.

The terms “block” or “blocking” refer to the ability of a compound to prevent a biological interaction (e.g., binding) in a cell relative to a negative control, e.g., vehicle. For example, a compound can block ATP from binding to the ATP binding pocket of a kinase. Such blocking may occur by direct binding of the compound to the ATP binding pocket itself, or indirect blocking. In some embodiments, the term refers to a reduction in the level of binding of ATP to a kinase (e.g., BTK, HCK, LYN, SRC) to a level that is statistically significantly lower than an initial level, which may, for example, be a baseline level of ATP binding. In some embodiments, the term refers to a reduction in the level of ATP binding to a kinase (e.g., BTK, HCK, LYN, SRC) to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of ATP binding. In some embodiments, blocking ATP binding leads to a reduction in the level of enzyme activity (e.g., BTK, HCK, LYN, SRC activity) to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

When a compound or pharmaceutical composition is referred to as “selectively,” “specifically,” or “competitively” binding a first protein, the compound binds the first protein, e.g., BTK, HCK, LYN, or SRC, with a higher binding affinity (e.g., not less than about 2-fold, not less than about 5-fold, not less than about 10-fold, not less than about 30-fold, not less than about 100-fold, not less than about 1,000-fold, or not less than about 10,000-fold) than binding a second protein that is different from the first protein, e.g., BTK. In some embodiments, a compound blocks ATP binding to a first protein, e.g., HCK, LYN, or SRC, at a lower concentration (e.g., not less than about 2-fold, not less than about 5-fold, not less than about 10-fold, not less than about 30-fold, not less than about 100-fold, not less than about 1,000-fold, or not less than about 10,000-fold) than it blocks ATP binding a second protein that is different from the first protein, e.g., BTK.

Compounds which selectively block ATP binding to a kinase (e.g., BTK, HCK, LYN) provided herein can be identified and/or characterized by methods known in the art. Methods include purified enzyme and cell based biochemical and binding assays such as an HCK gatekeeper mutant rescue assay, an in vitro kinase assay, e.g., using HCK gatekeeper mutated kinase, competitive binding assays using KiNativ technology or biotin tagged inhibitors, e.g., HCK inhibitors. Suitable assays for determining selective inhibition of HCK by a compound include, but are not limited to, Life Technology Z-Lyte activity assays (e.g., including HCK gatekeeper mutants and GK+6 mutants); DiscoverX KinomeScan binding assays; MRC radioactivity assays; ACD Ba/F₃ viability assays (e.g., including HCK gatekeeper mutants and GK+6 mutants); Yeast hybrid proliferation assays; Protein thermostability assays; and cancer cells with HCK gatekeeper mutants or GK+6 mutants proliferation-rescue assays. Such assays can also be used to determine selective inhibition of LYN and/or SRC by a compound.

The term “MYD88 mutation” means any change or difference in the nucleic acid or protein sequence of MYD88 as compared to the wild type sequence that results in the activation of MYD88 which leads to the activation of NF-κB. Mutations include, but are not limited to, nonsense mutations, missense mutations, frameshift mutations, rearrangement mutations, insertion mutations, and deletion mutations. In some embodiments, the mutation is a somatic mutation at position 38182641 in chromosome 3p22.2 which results in a single nucleotide change from T→C in the myeloid differentiation primary response (MYD88) gene, and a predicted non-synonymous change at amino acid position 265 from leucine to proline (L265P). In some embodiments, the mutation is another activating mutation in MYD88, such as V217F, W218R, I220T, S222R, M232T, S243N, T294P. Signaling studies show that SU-DHL-2 lymphoma cells that express the serine to arginine mutation at amino acid position 222 also have upregulated HCK (Yang et al., Blood 2016). In some embodiments, Sanger sequencing, whole exome or whole genome sequencing can be used to identify somatic mutations in MYD88.

The term “MYD88 mutated disease” or “disease associated with mutated MYD88” means any disease in a subject that is related to a change or difference in the nucleic acid or protein sequence of MYD88 as compared to the wild type sequence that results in the activation of MYD88 which leads to the activation of NF-1B. In some embodiments, mutated MYD88 disease is associated with a cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), and leukemia (e.g., chronic lymphocytic leukemia (CLL), acute lymphoblastic leukemia, myelogenous leukemia (e.g., chronic myelogenous leukemia, acute myelogenous leukemia))))). In some embodiments, mutated MYD88 is associated with susceptibility to an infectious disease. In some embodiments, mutated MYD88 is associated with susceptibility to an autoimmune disease

### BRIEF DESCRIPTION OF THE DRAWINGS

FIG. 1 shows the mean tumor volume during a rat efficacy study for Compound (I-1) in MYD88 mutated ABC DLBCL TMD8 xenografted rat model.

FIG. 2 shows the survival proportions during a rat efficacy study for Compound (I-1) in MYD88 mutated ABC DLBCL TMD8 xenografted rat model.

FIG. 3 shows the mean tumor volume during a rat efficacy study for Compound (I-1) in MYD88 mutated ABC DLBCL TMD8 xenografted rat model.

FIG. 4 shows the plot of the Invitrogen kinase assay for Compound (I-1) across several different cell lines, indicated with labels A-I.

FIG. 5 shows the plot of the Invitrogen kinase assay for Compound (I-12) across several different cell lines, indicated with labels A-F.

FIG. 6 shows the plot of the Invitrogen kinase assay for Compound (I-4) across several different cell lines, indicated with labels A-I.

FIG. 7 shows the plot of the Invitrogen kinase assay for Compound (I-16) across several different cell lines, indicated with labels A-H.

FIG. 8 shows the structure of TL2-059 (See: PCT publication WO 2011/090738 for synthesis details).

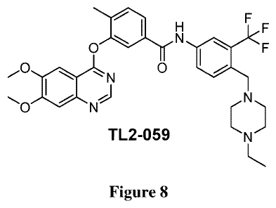

### DETAILED DESCRIPTION OF CERTAIN EMBODIMENTS OF THE INVENTION

Kinases are implicated in a wide variety of diseases, e.g., proliferative diseases. Provided herein are compounds of Formula (I), and pharmaceutically acceptable salts, solvates, hydrates, polymorphs, co-crystals, tautomers, stereoisomers, isotopically labeled derivatives, and prodrugs thereof. The provided compounds may be kinase inhibitors. In certain embodiments, the kinase being targeted is an SRC family kinase (e.g., HCK, LYN). In certain embodiments, the kinase being targeted is a Tec family kinase (e.g., BTK). Also provided are pharmaceutical compositions and kits comprising the provided compounds. Further provided are methods of using the compounds, pharmaceutical compositions, and kits for treating a disease (e.g., proliferative disease) in a subject in need thereof. In certain embodiments, the disease is a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))). Further provided are methods of using the provided compounds, pharmaceutical compositions, and kits for inhibiting the activity of a kinase in a subject in need thereof, in a biological sample, or in a cell.

### Compounds

In one aspect of the present disclosure, provided herein are compounds of Formula (I):

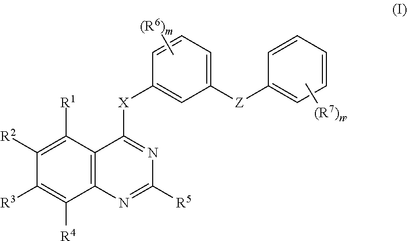

or a pharmaceutically acceptable salt, solvate, hydrate, polymorph, co-crystal, tautomer, stereoisomer, isotopically labeled compound, or prodrug thereof, wherein:

R¹, R², R³, R⁴, and R⁵ are each independently hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —N(R^{d})₂, —OR^{a}, —SR^{a}, —CN, —SCN, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
provided that at least one of R¹, R², R³, and R⁴ is —N(R^{d})₂, —NR^{a}C(═O)R^{a}, —NR^{a}C(═O)OR^{a}, or —NR^{a}C(═O)N(R^{a})₂;
each instance of R^{a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on the same nitrogen atom are joined to form substituted or unsubstituted heterocyclyl or substituted or unsubstituted heteroaryl;
each instance of R^{d} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group; or two instances of R^{d} on the same nitrogen atom are joined to form substituted or unsubstituted heterocyclyl or substituted or unsubstituted heteroaryl; or one instance of R^{d} is joined with one of R¹, R², R³, R⁴, or the other instance of R^{d} to form an optionally substituted heterocyclyl or optionally substituted heteroaryl;
R⁶ and R⁷ are each independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}—CN, —SCN, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, or —OC(═O)N(R^{b})₂;
each instance of R^{b} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{b} on the same nitrogen atom are joined to form substituted or unsubstituted heterocyclyl or substituted or unsubstituted heteroaryl;
m is 0, 1, 2, 3, or 4;
n is 0, 1, 2, 3, 4, or 5;
X is —N(R^{b})—, —O—, or —S—;
Z is —C(═O)N(R^{c})—, —N(R^{c})C(═O)—, —N(R^{c})C(═NR^{c})—, —C(═O)O—, or —C(═O)—; and
R^{c} is hydrogen, optionally substituted alkyl, optionally substituted acyl, or a nitrogen protecting group.

Formula (I), as described herein, contains the substituents R¹, R², R³, R⁴, and R⁵, wherein at least one of R¹, R², R³, and R⁴ is —N(R^{d})₂, —NR^{a}C(═O)R^{a}, —NR^{a}C(═O)OR^{a}, or —NR^{a}C(═O)N(R^{a})₂. In certain embodiments, at least on instance of R¹, R², R³, R⁴, or R⁵ is independently hydrogen. In certain embodiments, at least on instance of R¹, R², R³, R⁴, or R⁵ is independently halogen. In certain embodiments, at least on instance of R¹, R², R³, R⁴, or R⁵ is independently optionally substituted acyl. In certain embodiments, at least on instance of R¹, R², R³, R⁴, or R⁵ is independently optionally substituted alkyl. In certain embodiments, at least on instance of R¹, R², R³, R⁴, or R⁵ is independently optionally substituted alkenyl. In certain embodiments, at least on instance of R¹, R², R³, R⁴, or R⁵ is independently optionally substituted alkynyl. In certain embodiments, at least on instance of R¹, R², R³, R⁴, or R⁵ is independently optionally substituted carbocyclyl. In certain embodiments, at least on instance of R¹, R², R³, R⁴, or R⁵ is independently optionally substituted heterocyclyl. In certain embodiments, at least on instance of R¹, R², R³, R⁴, or R⁵ is independently optionally substituted aryl, optionally substituted heteroaryl. In certain embodiments, at least on instance of R¹, R², R³, R⁴, or R⁵ is independently —N(R^{d})₂. In certain embodiments, at least on instance of R¹, R², R³, R⁴, or R⁵ is independently —OR^{a}. In certain embodiments, at least on instance of R¹, R², R³, R⁴, or R⁵ is independently —SR^{a}. In certain embodiments, at least on instance of R¹, R², R³, R⁴, or R⁵ is independently —CN. In certain embodiments, at least on instance of R¹, R², R³, R⁴, or R⁵ is independently —SCN. In certain embodiments, at least on instance of R¹, R², R³, R⁴, or R⁵ is independently —C(═O)R^{a}. In certain embodiments, at least on instance of R¹, R², R³, R⁴, or R⁵ is independently —C(═O)OR^{a}. In certain embodiments, at least on instance of R¹, R², R³, R⁴, or R⁵ is independently —C(═O)N(R^{a})₂. In certain embodiments, at least on instance of R¹, R², R³, R⁴, or R⁵ is independently —NO₂. In certain embodiments, at least on instance of R¹, R², R³, R⁴, or R⁵ is independently —NR^{a}C(═O)R^{a}. In certain embodiments, at least on instance of R¹, R², R³, R⁴, or R⁵ is independently —NR^{a}C(═O)OR^{a}. In certain embodiments, at least on instance of R¹, R², R³, R⁴, or R⁵ is independently —NR^{a}C(═O)N(R^{a})₂. In certain embodiments, at least on instance of R¹, R², R³, R⁴, or R⁵ is independently —OC(═O)R^{a}. In certain embodiments, at least on instance of R¹, R², R³, R⁴, or R⁵ is independently —OC(═O)OR^{a}. In certain embodiments, at least on instance of R¹, R², R³, R⁴, or R⁵ is independently —OC(═O)N(R^{a})₂.

In certain embodiments, R¹ is H.

In certain embodiments, R⁴ is H.

In certain embodiments, R⁵ is H.

In certain embodiments, R¹ is H, and R⁴ is H. In certain embodiments, R¹ is H, and R⁵ is H. In certain embodiments, R⁴ is H, and R⁵ is H. In certain embodiments, R¹ is H, R⁴ is H, and R⁵ is H. In certain embodiments, R¹ is H, R² is H, R⁴ is H, and R⁵ is H.

In certain embodiments, R² is —OR^{a}, —OC(═O)R^{a}, —OC(═O)OR^{a}, or —OC(═O)N(R^{a})₂. In certain embodiments, R² is —OR^{a}. In certain embodiments, R² is —N(R^{d})₂. In certain embodiments, R² is —NR^{a}C(═O)R^{a}. In certain embodiments, R² is —NR^{a}C(═O)OR^{a}. In certain embodiments, R² is —NR^{a}C(═O)N(R^{a})₂. In certain embodiments, R² is H.

In certain embodiments, R³ is —N(R^{d})₂, —NR^{a}C(═O)R^{a}, —NR^{a}C(═O)OR^{a}, or —NR^{a}C(═O)N(R^{a})₂. In certain embodiments, R³ is —OR^{a}, —OC(═O)R^{a}, —OC(═O)OR^{a}, or —OC(═O)N(R^{a})₂.

In certain embodiments, R² is —N(R^{d})₂, —NR^{a}C(═O)R^{a}, —NR^{a}C(═O)OR^{a}, or —NR^{a}C(═O)N(R^{a})₂; and R³ is —OR^{a}, —OC(═O)R^{a}, —OC(═O)OR^{a}, or —OC(═O)N(R^{a})₂.

In certain embodiments, R² is —N(R^{d})₂, —NR^{a}C(═O)R^{a}, —NR^{a}C(═O)OR^{a}, or —NR^{a}C(═O)N(R^{a})₂; and R³ is —OR^{a}.

In certain embodiments, R² is —OR^{a}, —OC(═O)R^{a}, —OC(═O)OR^{a}, or —OC(═O)N(R^{a})₂; and R³ is —N(R^{d})₂, —NR^{a}C(═O)R^{a}, —NR^{a}C(═O)OR^{a}, or —NR^{a}C(═O)N(R^{a})₂.

In certain embodiments, R² is —OR^{a}; and R³ is —N(R^{d})₂, —NR^{a}C(═O)R^{a}, —NR^{a}C(═O)OR^{a}, or —NR^{a}C(═O)N(R^{a})₂.

In certain embodiments, at least one R^{a} is hydrogen. In certain embodiments, at least one R^{a} is substituted or unsubstituted alkyl. In certain embodiments, at least one R^{a} is substituted or unsubstituted alkenyl. In certain embodiments, at least one R^{a} is substituted or unsubstituted alkynyl. In certain embodiments, at least one R^{a} is substituted or unsubstituted carbocyclyl. In certain embodiments, at least one R^{a} is substituted or unsubstituted heterocyclyl. In certain embodiments, at least one R^{a} is substituted or unsubstituted aryl. In certain embodiments, at least one R^{a} is substituted or unsubstituted heteroaryl. In certain embodiments, at least one R^{a} is a nitrogen protecting group when attached to a nitrogen atom. In certain embodiments, at least one R^{a} is an oxygen protecting group when attached to an oxygen atom. In certain embodiments, at least one R^{a} is or a sulfur protecting group when attached to a sulfur atom. In certain embodiments, two instances of R^{a} on the same nitrogen atom are joined to form substituted or unsubstituted heterocyclyl or substituted or unsubstituted heteroaryl.

In certain embodiments, at least one R^{a} is substituted or unsubstituted alkyl. In certain embodiments, at least one R^{a} is optionally substituted C₁-C₆ alkyl. In certain embodiments, at least one R^{a} is optionally substituted methyl. In certain embodiments, at least one R^{a} is unsubstituted methyl.

In certain embodiments, at least one R^{d} is hydrogen. In certain embodiments, at least one R^{d} is substituted or unsubstituted alkyl. In certain embodiments, at least one R^{d} is substituted or unsubstituted alkenyl. In certain embodiments, at least one R^{d} is substituted or unsubstituted alkynyl. In certain embodiments, at least one R^{d} is substituted or unsubstituted carbocyclyl. In certain embodiments, at least one R^{d} is substituted or unsubstituted heterocyclyl. In certain embodiments, at least one R^{d} is substituted or unsubstituted aryl. In certain embodiments, at least one R^{d} is substituted or unsubstituted heteroaryl. In certain embodiments, at least one R^{d} is a nitrogen protecting group. In certain embodiments, two instances of R^{d} on the same nitrogen atom are joined to form substituted or unsubstituted heterocyclyl. In certain embodiments, two instances of R^{d} are joined to form a substituted or unsubstituted heteroaryl. In certain embodiments, one instance of R^{d} is joined with one of R¹, R², R³, R⁴, or another instance of R^{d} to form an optionally substituted heterocyclyl. In certain embodiments, one instance of R^{d} is joined with one of R¹, R², R³, R⁴, or another instance of R^{d} to form an optionally substituted heteroaryl.

In certain embodiments, at least one R^{d} is substituted or unsubstituted alkyl. In certain embodiments, at least one R^{d} is optionally substituted C₁-C₆ alkyl. In certain embodiments, at least one R^{d} is optionally substituted methyl. In certain embodiments, at least one R^{d} is unsubstituted methyl.

In certain embodiments, at least one R⁶ is halogen. In certain embodiments, at least one R⁶ is optionally substituted acyl. In certain embodiments, at least one R⁶ is optionally substituted alkyl. In certain embodiments, at least one R⁶ is optionally substituted alkenyl. In certain embodiments, at least one R⁶ is optionally substituted alkynyl. In certain embodiments, at least one R⁶ is optionally substituted carbocyclyl. In certain embodiments, at least one R⁶ is optionally substituted heterocyclyl. In certain embodiments, at least one R⁶ is optionally substituted aryl. In certain embodiments, at least one R⁶ is optionally substituted heteroaryl. In certain embodiments, at least one R⁶ is —OR^{b}. In certain embodiments, at least one R⁶ is —N(R^{b})₂. In certain embodiments, at least one R⁶ is —SR^{b}. In certain embodiments, at least one R⁶ is —CN. In certain embodiments, at least one R⁶ is —SCN. In certain embodiments, at least one R⁶ is —C(═O)R^{b}. In certain embodiments, at least one R⁶ is —C(═O)OR^{b}. In certain embodiments, at least one R⁶ is —C(═O)N(R^{b})₂. In certain embodiments, at least one R⁶ is —NO₂. In certain embodiments, at least one R⁶ is —NR^{b}C(═O)R^{b}. In certain embodiments, at least one R⁶ is —NR^{b}C(═O)OR^{b}. In certain embodiments, at least one R⁶ is —NR^{b}C(═O)N(R^{b})₂. In certain embodiments, at least one R⁶ is —OC(═O)R^{b}. In certain embodiments, at least one R⁶ is —OC(═O)OR^{b}, In certain embodiments, at least one R⁶ is —OC(═O)N(R^{b})₂.

In certain embodiments, at least one R⁷ is halogen. In certain embodiments, at least one R⁷ is optionally substituted acyl. In certain embodiments, at least one R⁷ is optionally substituted alkyl. In certain embodiments, at least one R⁷ is optionally substituted alkenyl. In certain embodiments, at least one R⁷ is optionally substituted alkynyl. In certain embodiments, at least one R⁷ is optionally substituted carbocyclyl. In certain embodiments, at least one R⁷ is optionally substituted heterocyclyl. In certain embodiments, at least one R⁷ is optionally substituted aryl. In certain embodiments, at least one R⁷ is optionally substituted heteroaryl. In certain embodiments, at least one R⁷ is —OR^{b}. In certain embodiments, at least one R⁷ is —N(R^{b})₂. In certain embodiments, at least one R⁷ is —SR^{b}. In certain embodiments, at least one R⁷ is —CN. In certain embodiments, at least one R⁷ is —SCN. In certain embodiments, at least one R⁷ is —C(═O)R^{b}. In certain embodiments, at least one R⁷ is —C(═O)OR^{b}. In certain embodiments, at least one R⁷ is —C(═O)N(R^{b})₂. In certain embodiments, at least one R⁷ is —NO₂. In certain embodiments, at least one R⁷ is —NR^{b}C(═O)R^{b}. In certain embodiments, at least one R⁷ is —NR^{b}C(═O)OR^{b}. In certain embodiments, at least one R⁷ is —NR^{b}C(═O)N(R^{b})₂. In certain embodiments, at least one R⁷ is —OC(═O)R^{b}. In certain embodiments, at least one R⁷ is —OC(═O)OR^{b}, In certain embodiments, at least one R⁷ is —OC(═O)N(R^{b})₂.

In certain embodiments, at least one R^{b} is hydrogen. In certain embodiments, at least one R^{b} is optionally substituted acyl. In certain embodiments, at least one R^{b} is optionally substituted alkyl. In certain embodiments, at least one R^{b} is optionally substituted alkenyl. In certain embodiments, at least one R^{b} is optionally substituted alkynyl. In certain embodiments, at least one R^{b} is optionally substituted carbocyclyl. In certain embodiments, at least one R^{b} is optionally substituted heterocyclyl. In certain embodiments, at least one R^{b} is optionally substituted aryl. In certain embodiments, at least one R^{b} is optionally substituted heteroaryl. In certain embodiments, at least one R^{b} is a nitrogen protecting group when attached to a nitrogen atom. In certain embodiments, at least one R^{b} is an oxygen protecting group when attached to an oxygen atom. In certain embodiments, at least one R^{b} is a sulfur protecting group when attached to a sulfur atom. In certain embodiments, two instances of R^{b} on the same nitrogen atom are joined to form substituted or unsubstituted heterocyclyl or substituted or unsubstituted heteroaryl.

In certain embodiments, at least one R⁶ is optionally substituted alkyl. In certain embodiments, at least one R⁶ is optionally substituted methyl. In certain embodiments, at least one R⁶ is unsubstituted methyl.

In certain embodiments, at least one instance of R⁷ is R^{7a}. In certain embodiments, at least one R⁷ is R^{7b}. In certain embodiments, at least one instance of R⁷, R^{7a}, or R^{7b} is optionally substituted alkyl.

In certain embodiments, at least one instance of R⁷, R^{7a}, or R^{7b} is of the formula:

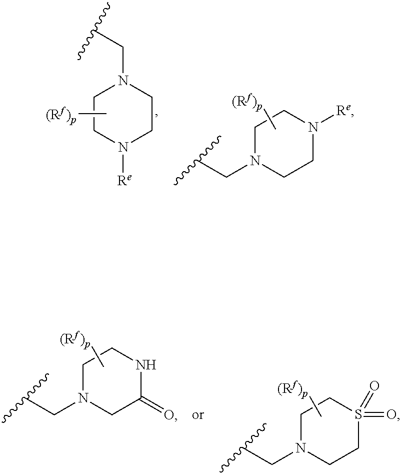

wherein R^{e} is hydrogen, substituted or unsubstituted alkyl, or substituted or unsubstituted acyl, or a nitrogen protecting group;
each instance of R^{f} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}—CN, —SCN, —C(═O)R^{g}, —C(═O)OR^{g}, —C(═O)N(R^{g})₂, —NO₂, —NR^{g}C(═O)R^{g}, —NR^{g}C(═O)OR^{g}, —NR^{g}C(═O)N(R^{g})₂, —OC(═O)R^{g}, —OC(═O)OR^{g}, or —OC(═O)N(R^{g})₂, wherein each instance of R^{g} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom; and
p is 0, 1, 2, 3, 4, 5, 6, 7, or 8.

In certain embodiments, R^{e} is hydrogen. In certain embodiments, R^{e} is substituted or unsubstituted alkyl. In certain embodiments, R^{e} is substituted or unsubstituted acyl. In certain embodiments, R^{e} is a nitrogen protecting group

In certain embodiments, at least one instance of R⁷, R^{7a}, or R^{7b} is of the formula:

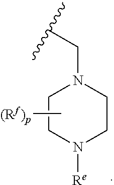

In certain embodiments, at least one instance of R⁷, R^{7a}, or R^{7b} is of the formula:

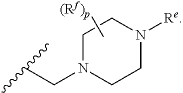

In certain embodiments, at least one instance of R⁷, R^{7a}, or R^{7b} is of the formula:

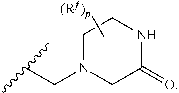

In certain embodiments, at least one instance of R⁷, R^{7a}, or R^{7b} is of the formula:

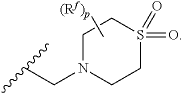

In certain embodiments, at least one instance of R⁷, R^{7a}, or R^{7b} is of the formula:

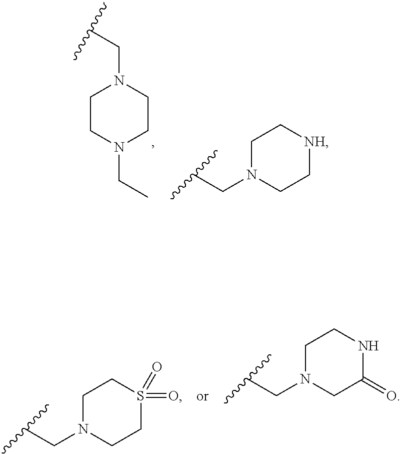

In certain embodiments, at least one instance of R⁷, R^{7a}, or R^{7b} is of the formula:

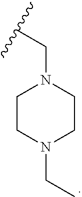 [SMILES: *CN1CCN(CC)CC1]

In certain embodiments, at least one instance of R⁷, R^{7a}, or R^{7b} is of the formula:

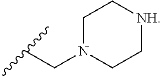 [SMILES: *CN1CCNCC1]

In certain embodiments, at least one instance of R⁷, R^{7a}, or R^{7b} is of the formula:

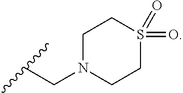 [SMILES: *CN1CCS(=O)(=O)CC1]

In certain embodiments, at least one instance of R⁷, R^{7a}, or R^{7b} is of the formula:

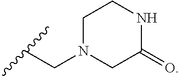 [SMILES: *CN1CCNC(=O)C1]

In certain embodiments, at least one instance of R⁷, R^{7a}, or R^{7b} is C₁₋₆ perhaloalkyl. In certain embodiments, at least one instance of R⁷, R^{7a}, or R^{7b} is —CF₃.

In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8.

In certain embodiments, X is —N(R^{b})—, —O—, or —S—. In certain embodiments, X is —O—.

In certain embodiments, Z is —C(═O)N(R^{c})—, —N(R^{c})C(═O)—, —N(R^{c})C(═NR^{c})—, —C(═O)O—, or —C(═O)—. In certain embodiments, Z is —C(═O)N(R^{c})—. In certain embodiments, Z is —C(═O)N(H)—.

In certain embodiments, R^{c} is hydrogen, optionally substituted alkyl, optionally substituted acyl, or a nitrogen protecting group. In certain embodiments, R^{c} is H.

In certain embodiments, the compound of Formula (I) is of the Formula (II):

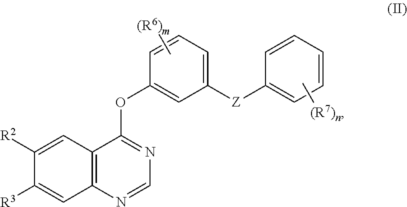

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (III):

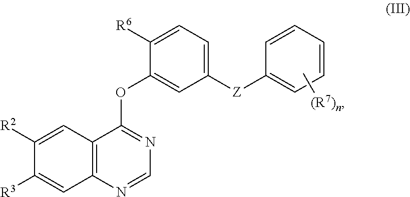

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (IV):

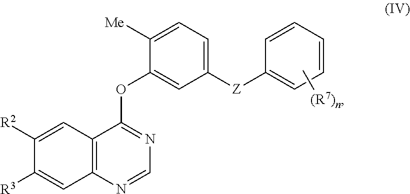

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (V):

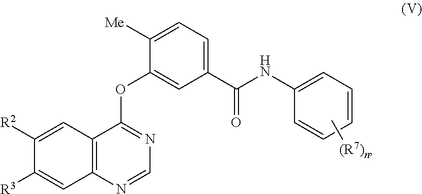

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (VI):

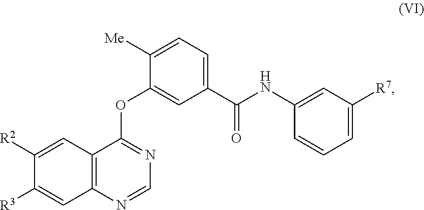

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (VII):

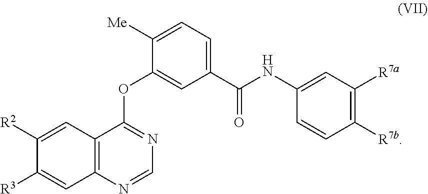

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (VI-a):

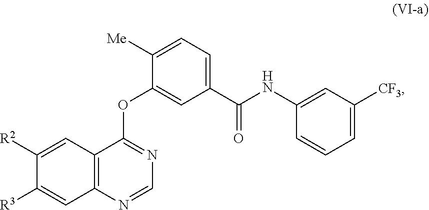

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (VII-a):

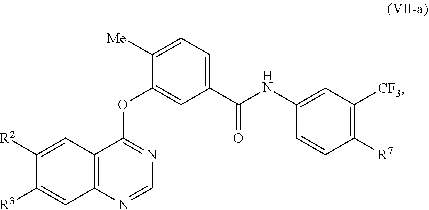

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (VII-b):

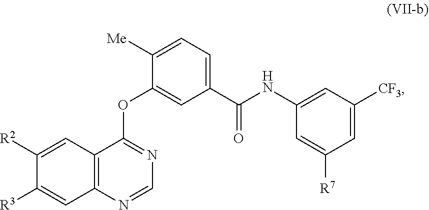

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (VII-c):

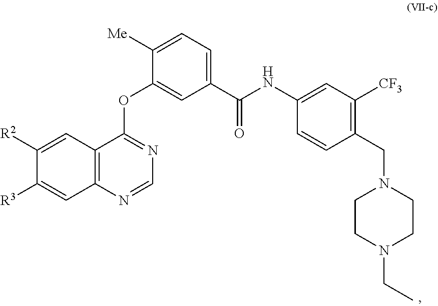

or a pharmaceutically acceptable salt, solvate, hydrate, polymorph, co-crystal, tautomer, stereoisomer, isotopically labeled compound, or prodrug thereof.

In certain embodiments, a compound of Formula (I) is a compound, or a pharmaceutically acceptable salt, solvate, hydrate, polymorph, co-crystal, tautomer, stereoisomer, isotopically labeled compound, or prodrug thereof, of the formula:

| # | Structure |
|---|---|
| (I-1) | 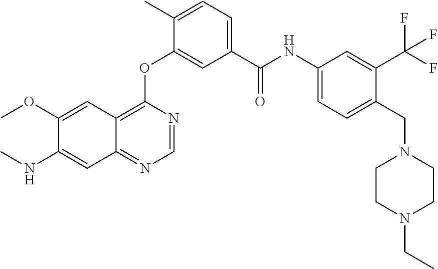 [SMILES: CCN1CCN(Cc2ccc(NC(=O)c3ccc(C)c(Oc4ncnc5cc(NC)c(OC)cc45)c3)cc2C(F)(F)F)CC1] |
| (I-2) | 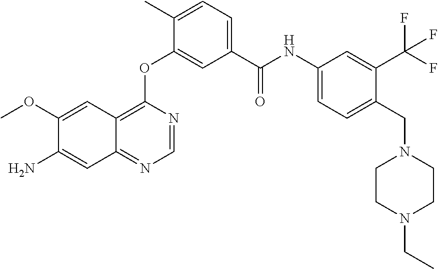 [SMILES: CCN1CCN(Cc2ccc(NC(=O)c3ccc(C)c(Oc4ncnc5cc(N)c(OC)cc45)c3)cc2C(F)(F)F)CC1] |
| (I-3) | 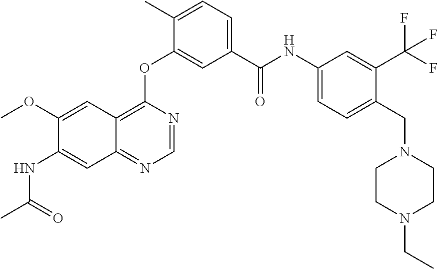 [SMILES: CCN1CCN(Cc2ccc(NC(=O)c3ccc(C)c(Oc4ncnc5cc(NC(C)=O)c(OC)cc45)c3)cc2C(F)(F)F)CC1] |
| (I-4) | 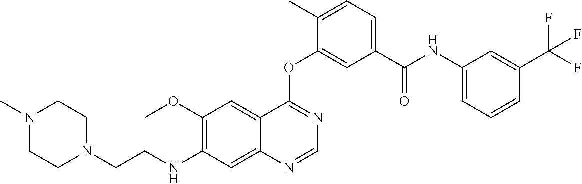 [SMILES: COc1cc2c(Oc3cc(C(=O)Nc4cccc(C(F)(F)F)c4)ccc3C)ncnc2cc1NCCN1CCN(C)CC1] |
| (I-5) | 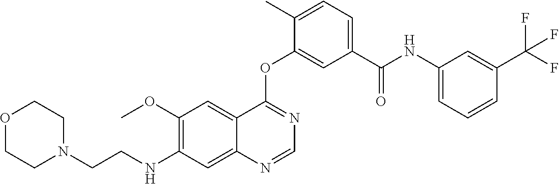 [SMILES: COc1cc2c(Oc3cc(C(=O)Nc4cccc(C(F)(F)F)c4)ccc3C)ncnc2cc1NCCN1CCOCC1] |
| (I-6) | 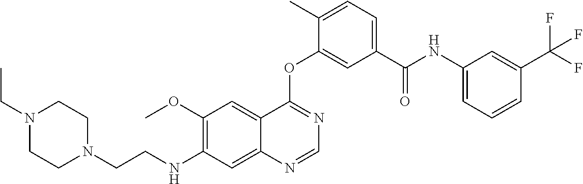 [SMILES: CCN1CCN(CCNc2cc3ncnc(Oc4cc(C(=O)Nc5cccc(C(F)(F)F)c5)ccc4C)c3cc2OC)CC1] |
| (I-7) | 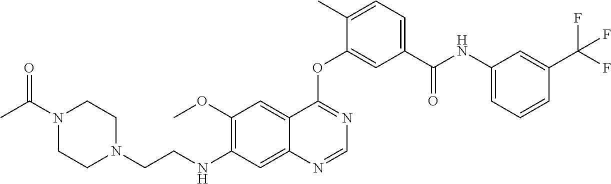 [SMILES: COc1cc2c(Oc3cc(C(=O)Nc4cccc(C(F)(F)F)c4)ccc3C)ncnc2cc1NCCN1CCN(C(C)=O)CC1] |
| (I-8) | 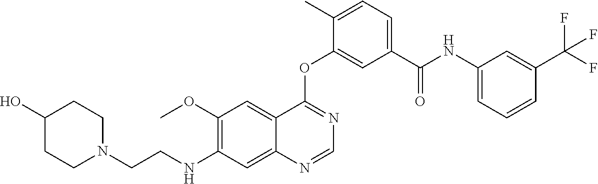 [SMILES: COc1cc2c(Oc3cc(C(=O)Nc4cccc(C(F)(F)F)c4)ccc3C)ncnc2cc1NCCN1CCC(O)CC1] |
| (I-9) | 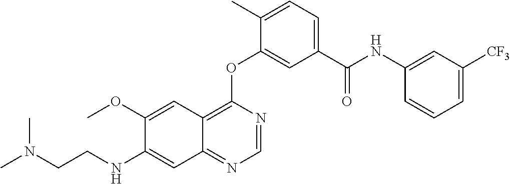 [SMILES: COc1cc2c(Oc3cc(C(=O)Nc4cccc(C(F)(F)F)c4)ccc3C)ncnc2cc1NCCN(C)C] |
| (I-10) | 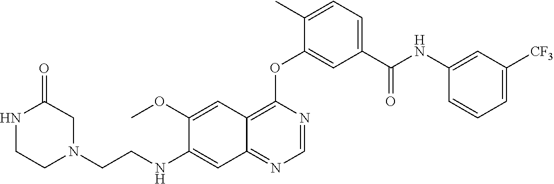 [SMILES: COc1cc2c(Oc3cc(C(=O)Nc4cccc(C(F)(F)F)c4)ccc3C)ncnc2cc1NCCN1CCNC(=O)C1] |
| (I-12) | 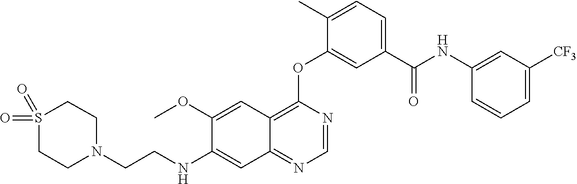 [SMILES: COc1cc2c(Oc3cc(C(=O)Nc4cccc(C(F)(F)F)c4)ccc3C)ncnc2cc1NCCN1CCS(=O)(=O)CC1] |
| (I-13) | 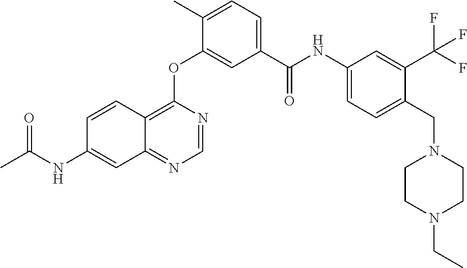 [SMILES: CCN1CCN(Cc2ccc(NC(=O)c3ccc(C)c(Oc4ncnc5cc(NC(C)=O)ccc45)c3)cc2C(F)(F)F)CC1] |
| (I-14) | 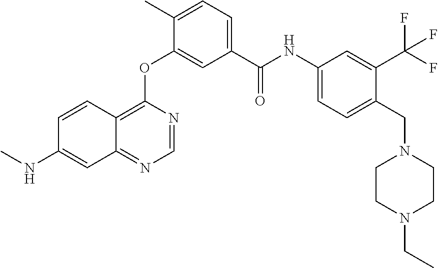 [SMILES: CCN1CCN(Cc2ccc(NC(=O)c3ccc(C)c(Oc4ncnc5cc(NC)ccc45)c3)cc2C(F)(F)F)CC1] |
| (I-15) | 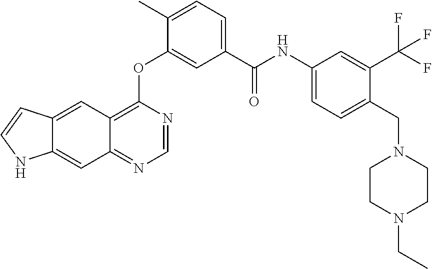 [SMILES: CCN1CCN(Cc2ccc(NC(=O)c3ccc(C)c(Oc4ncnc5cc6[nH]ccc6cc45)c3)cc2C(F)(F)F)CC1] |
| (I-16) | 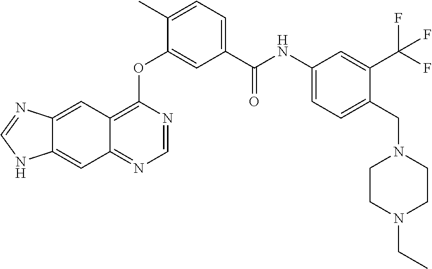 [SMILES: CCN1CCN(Cc2ccc(NC(=O)c3ccc(C)c(Oc4ncnc5cc6[nH]cnc6cc45)c3)cc2C(F)(F)F)CC1] |
| (I-17) | 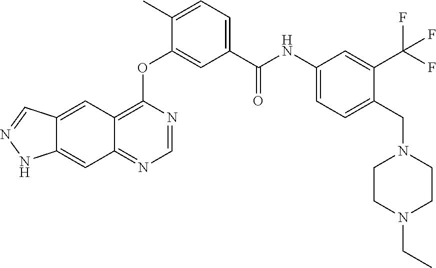 [SMILES: CCN1CCN(Cc2ccc(NC(=O)c3ccc(C)c(Oc4ncnc5cc6[nH]ncc6cc45)c3)cc2C(F)(F)F)CC1] |
| (I-18) | 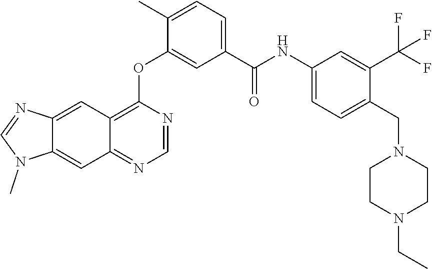 [SMILES: CCN1CCN(Cc2ccc(NC(=O)c3ccc(C)c(Oc4ncnc5cc6c(cc45)ncn6C)c3)cc2C(F)(F)F)CC1] |
| (I-19) | 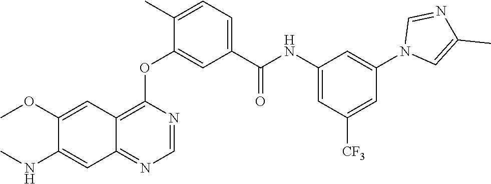 |
| (I-20) | 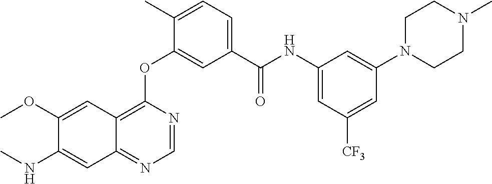 |
| (I-21) | 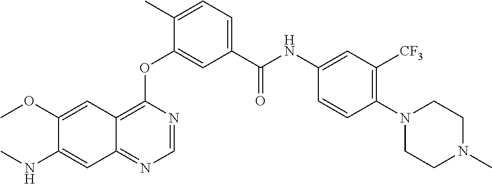 |
| (I-22) | 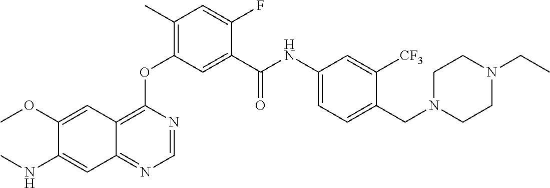 [SMILES: CCN1CCN(Cc2ccc(NC(=O)c3cc(Oc4ncnc5cc(NC)c(OC)cc45)c(C)cc3F)cc2C(F)(F)F)CC1] |
| (I-23) | 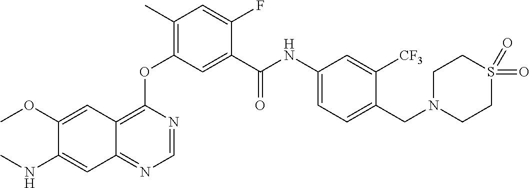 [SMILES: CNc1cc2ncnc(Oc3cc(C(=O)Nc4ccc(CN5CCS(=O)(=O)CC5)c(C(F)(F)F)c4)c(F)cc3C)c2cc1OC] |
| (I-24) | 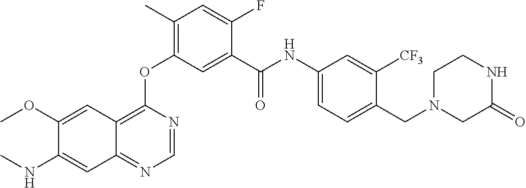 [SMILES: CNc1cc2ncnc(Oc3cc(C(=O)Nc4ccc(CN5CCNC(=O)C5)c(C(F)(F)F)c4)c(F)cc3C)c2cc1OC] |
| (I-25) | 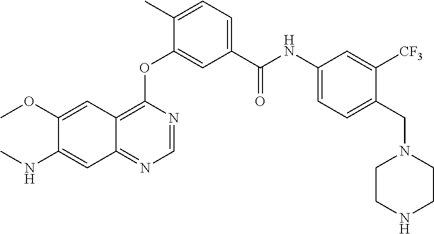 [SMILES: CNc1cc2ncnc(Oc3cc(C(=O)Nc4ccc(CN5CCNCC5)c(C(F)(F)F)c4)ccc3C)c2cc1OC] |
| (I-26) | 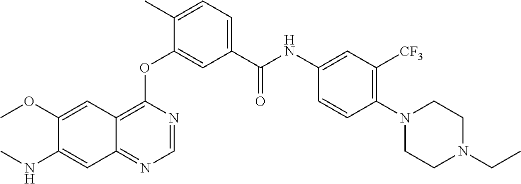 [SMILES: CCN1CCN(c2ccc(NC(=O)c3ccc(C)c(Oc4ncnc5cc(NC)c(OC)cc45)c3)cc2C(F)(F)F)CC1] |
| (I-27) | 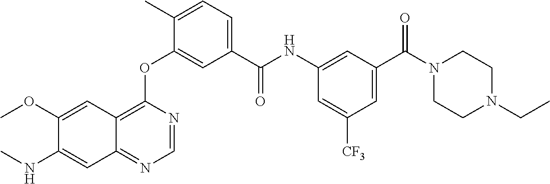 [SMILES: CCN1CCN(C(=O)c2cc(NC(=O)c3ccc(C)c(Oc4ncnc5cc(NC)c(OC)cc45)c3)cc(C(F)(F)F)c2)CC1] |

### Pharmaceutical Compositions, Administration, and Kits

The present disclosure also provides pharmaceutical compositions comprising a compound described herein and a pharmaceutically acceptable excipient. In certain embodiments, the pharmaceutical composition further comprises an additional pharmaceutical agent.

In certain embodiments, the compound described herein is provided in an effective (e.g., effective for inhibiting a kinase, such as BTK, HCK, LYN) amount in the pharmaceutical composition. In certain embodiments, the effective amount is a therapeutically effective amount. In certain embodiments, a therapeutically effective amount is an amount effective for inhibiting a kinase. In certain embodiments, a therapeutically effective amount is an amount effective for treating a disease (e.g., a disease associated with aberrant activity of a kinase (e.g., proliferative disease)). In certain embodiments, a therapeutically effective amount is an amount effective for inhibiting the activity of a kinase and treating a disease (e.g., a disease associated with aberrant activity of a kinase (e.g., proliferative disease (e.g., cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), and leukemia (e.g., chronic lymphocytic leukemia (CLL), acute lymphoblastic leukemia, myelogenous leukemia (e.g., chronic myelogenous leukemia, acute myelogenous leukemia))))).

In certain embodiments, the effective amount is an amount effective for inhibiting the activity of a kinase by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a kinase by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human (e.g., an adult, juvenile, or child). In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a dog. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the subject is a genetically engineered animal. In certain embodiments, the subject is a transgenic animal (e.g., transgenic mice, transgenic pigs). In certain embodiments, the subject is a fish or reptile.

In certain embodiments, the subject is a human. In certain embodiments, the human is an adult. In certain embodiments, the human is a juvenile. In certain embodiments, the human is a child.

In certain embodiments, the biological sample or cell (e.g., the biological sample or cell being contacted with a compound or pharmaceutical composition described herein) is in vitro. In certain embodiments, the biological sample or cell is in vivo or ex vivo. In certain embodiments, the cell is a malignant cell or premalignant cell.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragées, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample or cell, any two doses of the multiple doses include different or substantially the same amounts of a compound described herein. In certain embodiments, when multiple doses are administered to a subject or applied to a biological sample or cell, the frequency of administering the multiple doses to the subject or applying the multiple doses to the biological sample or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample or cell is one dose per day. In certain embodiments, the frequency of administering the multiple doses to the subject or applying the multiple doses to the biological sample or cell is two doses per day. In certain embodiments, the frequency of administering the multiple doses to the subject or applying the multiple doses to the biological sample or cell is three doses per day. In certain embodiments, when multiple doses are administered to a subject or applied to a biological sample or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject or cell. In certain embodiments, the duration between the first dose and last dose of the multiple doses is three months, six months, or one year. In certain embodiments, the duration between the first dose and last dose of the multiple doses is the lifetime of the subject or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity of a kinase (e.g., BTK, HCK, LYN) in a subject, biological sample, or cell), improve bioavailability, improve safety, reduce drug resistance, reduce and/or modify metabolism, inhibit excretion, and/or modify distribution in a subject, biological sample, or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, cancer, inflammatory disease, autoimmune disease, genetic disease, hematological disease, neurological disease, painful condition, psychiatric disorder, or metabolic disorder) or premalignant condition. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, cytotoxic chemotherapeutic agents, epigenetic modifiers, glucocorticoids, immunotherapeutic agents, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmazine), BELEODAQ (belinostat), BEXXAR (tositumomab and iodine I131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOLVADEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055, BEZ235, BGT226, XL765, PF-4691502, GDC0980, SF1126, and OSI-027), oblimersen, gemcitabine, carminomycin, leucovorin, pemetrexed, cyclophosphamide, dacarbazine, procarbazine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof.

In certain embodiments, the additional pharmaceutical agent is a cytotoxic chemotherapeutic agent (e.g., gemcitabine, cytarabine, daunorubicin, doxorubicin, vincristine, 1-asparaginase, cyclophosphamide, or etoposide). In certain embodiments, the additional pharmaceutical agent is an epigenetic modifier such as azacitidine or romidepsin. In certain embodiments, the additional pharmaceutical agent is ruxolitinib, BBT594, CHZ868, CYT387, or BMS911543. In certain embodiments, the additional pharmaceutical agent is an inhibitor of a tyrosine kinase. In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In certain embodiments, the additional pharmaceutical agent is a BCL-2 inhibitor. In certain embodiments, the additional pharmaceutical agent is venetoclax. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. In certain embodiments, the additional pharmaceutical agent is a binder or inhibitor of a kinase (e.g., JAK, ABL1, CDC2L1, CDC2L2, CSF1R, DDR1, DDR2, FLT4, KIT, PDGFRB, RET, TAOK2, or a combination thereof). In certain embodiments, the additional pharmaceutical agent is an antibody or a fragment thereof (e.g., monoclonal antibody). In certain embodiments, the additional pharmaceutical agent is a tyrosine kinase inhibitor. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the additional pharmaceutical agent is a glucocorticoid (e.g., cortisol, cortisone, prednisone, methylprednisolone, dexamethasone, betamethasone, triamcinolone, fludrocortisone acetate, or deoxycorticosterone acetate). In certain embodiments, the additional therapy is an immunotherapy (e.g., an immunotherapeutic monoclonal antibody). In certain embodiments, the additional pharmaceutical agent is an immunomodulator. In certain embodiments, the additional pharmaceutical agent is an immune checkpoint inhibitor. In certain embodiments, the additional pharmaceutical agent is a programmed cell death 1 protein (PD-1) inhibitor. In certain embodiments, the additional pharmaceutical agent is a programmed cell death 1 protein ligand 1 (PD-L1) inhibitor. In certain embodiments, the additional pharmaceutical agent is a cytotoxic T-lymphocyte-associated protein 4 (CTLA-4) inhibitor. In certain embodiments, the additional pharmaceutical agent is a BTK inhibitor (e.g., ibrutinib, CC-292, ONO-4059, evobrutinib, spebrutinib, BGB-3111, HM71224, or ACP-196 (i.e., acalabrutinib)). In certain embodiments, the compounds described herein or pharmaceutical compositions can be administered in combination with an anti-cancer therapy including, but not limited to, surgery, radiation therapy, and transplantation (e.g., stem cell transplantation, bone marrow transplantation).

In certain embodiments, the additional pharmaceutical agent is a BCL-2 inhibitor (e.g., venetoclax, navitoclax, obatoclax), a BCL-2/BCL-xL inhibitor (e.g., APG-1252, BM-1197).

In certain embodiments, the additional pharmaceutical agent is venetoclax. In certain embodiments, the additional pharmaceutical agent is navitoclax. In certain embodiments, the additional pharmaceutical agents is obatoclax.

Also encompassed by the present disclosure are kits (e.g., pharmaceutical packs). In certain embodiments, the kit comprises a compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the compound or pharmaceutical composition included in the first container and the excipient or additional pharmaceutical agent included in the second container are combined to form one unit dosage form. In some embodiments, the compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the compound or pharmaceutical composition to a subject (e.g., a subject in need of treatment or prevention of a disease described herein). In certain embodiments, the instructions are for contacting a biological sample or cell with the compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

The compounds, pharmaceutical compositions, and kits described herein may synergistically augment inhibition of a kinase (e.g., BTK, HCK, LYN) induced by the additional pharmaceutical agent(s) in the biological sample or subject. Thus, the combination of the compounds, pharmaceutical compositions, or kits with additional pharmaceutical agent(s) may be useful in treating diseases resistant to a treatment using the additional pharmaceutical agent(s) without the compounds, pharmaceutical compositions, or kits described herein.

### Methods of Treatment and Uses

The present disclosure provides methods of modulating (e.g., inhibiting or increasing) the activity (e.g., aberrant activity, such as increased or decreased activity) of a kinase (e.g., BTK, HCK, LYN). The present disclosure also provides methods of modulating (e.g., inhibiting or increasing) the activity (e.g., undesired or aberrant activity, such as increased activity (e.g., activity above normal levels) or decreased activity (e.g., activity below normal levels)), of a kinase in a subject, biological sample, or cell. The present disclosure also provides methods for the treatment of a range of diseases and conditions, such as diseases and conditions associated with undesired or aberrant activity (e.g., increased activity) or overexpression of a kinase (e.g., BTK, HCK, LYN). In certain embodiments, the diseases include a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))).

In another aspect, the present disclosure provides methods of treating a disease in a subject in need thereof, the method comprising administering to the subject in need thereof an effective amount (e.g., therapeutically effective amount) of a compound described herein or a pharmaceutical composition described herein.

In another aspect, the present disclosure provides methods of preventing a disease in a subject in need thereof, the method comprising administering to the subject in need thereof an effective amount (e.g., prophylactically effective amount) of a compound described herein or a pharmaceutical composition described herein.

In another aspect, the present disclosure provides methods of inhibiting the activity of a kinase in a subject in need thereof, the method comprising administering to the subject in need thereof an effective amount of a compound described herein or a pharmaceutical composition described herein.

In another aspect, the present disclosure provides methods of inhibiting the activity of a kinase in a biological sample (e.g., an in vitro biological sample), the method comprising contacting the biological sample with an effective amount of a compound described herein or a pharmaceutical composition described herein.

In another aspect, the present disclosure provides methods of inhibiting the activity of a kinase in a cell (e.g., an in vitro cell), the method comprising contacting the cell with an effective amount of a compound described herein or a pharmaceutical composition described herein.

Without wishing to be bound by any particular theory, in certain embodiments the compounds described herein are able to bind (e.g., covalently modify) the kinase being inhibited. In certain embodiments, a compound described herein is able to bind (e.g., covalently modify) to the kinase. In certain embodiments, the kinase is HCK. In certain embodiments, the kinase is BTK. In certain embodiments, the kinase is LYN.

In certain embodiments, provided are methods of decreasing the activity of a kinase (e.g., HCK, BTK, LYN) in a subject, biological sample, or cell by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a kinase in a subject, biological sample, or cell is decreased by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a kinase in a subject, biological sample, or cell is selectively inhibited by the method. In some embodiments, the activity of a kinase (e.g., HCK, BTK, LYN) in a subject, biological sample, or cell is selectively decreased by a compound or pharmaceutical composition described herein.

A disease, including proliferative disease, may be associated with aberrant or undesired activity of a kinase, and/or overexpression of the kinase. Aberrant or undesired activity of a kinase may be an increased or a decreased level of activity of the kinase. Proliferative diseases are sometimes associated with abnormal levels of HCK, BTK, or LYN activity, frequently through increased or decreased HCK, BTK, or LYN activation. Inhibition of the activity of HCK, BTK, or LYN would be expected to inhibit phosphorylation. In certain embodiments, HCK, BTK, or LYN is not overexpressed, but the activity of HCK, BTK, or LYN is increased. In certain embodiments, HCK, BTK, or LYN is overexpressed, and the activity of HCK, BTK, or LYN is increased. The compounds and pharmaceutical compositions described herein may inhibit the activity of HCK, BTK, or LYN and be useful in treating and/or preventing diseases, such as diseases associated with the aberrant, increased, or undesired activity of a kinase, over activation of the kinase, and/or overexpression of the kinase.

In certain embodiments, the disease (e.g., the disease to be treated or prevented by a method described herein) is associated with the increased activity of a kinase (e.g., HCK, BTK, LYN). In certain embodiments, the disease is associated with overexpression of a kinase (e.g., HCK, BTK, LYN). In certain embodiments, the disease is a proliferative disease. In certain embodiments, the proliferative disease is cancer. In certain embodiments, the cancer is associated with a mutation in MYD88. In another embodiment, the cancer is associated with mutated BTK. In certain embodiments, the proliferative disease is mastocytosis. In certain embodiments, the mastocytosis is systemic mastocytosis. In certain embodiments, the proliferative disease is an IgM gammopathy. In certain embodiments, the IgM gammopathy is IgM monoclonal gammopathy with undetermined significance.

In certain embodiments, the cancer is breast cancer. In certain embodiments, the cancer is colon cancer. In certain embodiments, the cancer is testicular cancer. In certain embodiments, the cancer is CNS cancer. In certain embodiments, the cancer is stomach cancer. In certain embodiments, the cancer is lymphoma. In certain embodiments, the lymphoma is a B-cell Lymphoma. In certain embodiments, the B-cell lymphoma is lymphoplasmacytic lymphoma. In certain embodiments, the lymphoplasmacytic lymphoma is IgM secreting lymphoplasmacytic lymphoma (i.e., Waldenström's macroglobulinemia). In certain embodiments, the lymphoplasmacytic lymphoma is non-IgM secreting. In certain embodiments, the B-cell lymphoma is Diffuse Large B-Cell Lymphoma (DLBCL). In certain embodiments, the DLBCL is activated B-cell-like (ABC)-DLBCL. In certain embodiments, the DLBCL is germinal center B-cell-like (GBC)-DLBCL. In certain embodiments, the lymphoma is follicular lymphoma. In certain embodiments, the lymphoma is marginal zone B-cell lymphoma. In certain embodiments, the lymphoma is small lymphocytic lymphoma. In certain embodiments, the small lymphocytic lymphoma is mantle cell lymphoma. In certain embodiments, the cancer is leukemia. In certain embodiments, the leukemia is chronic lymphocytic leukemia (CLL). In certain embodiments, the leukemia is myelogenous leukemia. In certain embodiments, the myelogenous leukemia is chronic myelogenous leukemia. In certain embodiments, the myelogenous leukemia is acute myelogenous leukemia. In certain embodiments, the acute myelogenous leukemia is mast cell leukemia. In certain embodiments, the cancer is myeloma. In certain embodiments. the myeloma is IgM myeloma. In certain embodiments, the IgM myeloma is IgM multiple myeloma. In certain embodiments, the cancer is a myeloproliferative disease. In certain embodiments, the myeloproliferative disease is myelodysplastic syndrome.

Further provided herein is a method of treating a subject comprising administering to a subject with an MYD88 mutated disease. An MYD88 mutated disease can include, but is not limited to a proliferative disease (e.g., an IgM gammopathy (e.g., an IgM Monoclonal gammopathy of undetermined significance (MGUS), amyloid light chain (AL) amyloidosis), mastocytosis (e.g., systemic mastocytosis) cancer (e.g., breast cancer, colon cancer, testicular cancer, CNS cancer, stomach cancer, lymphoma (e.g., B-cell lymphoma (e.g., lymphoplasmacytic lymphoma (e.g., IgM secreting lymphoplasmacytic lymphoma (i.e., Waldenström's Macroglobulinemia), non-IgM secreting lymphoplasmacytic lymphoma)), diffuse large B-cell lymphoma (e.g., activated B-cell-like (ABC)-DLBCL, germinal center B-cell-like (GBC)-DLBCL), follicular lymphoma, marginal zone B-cell lymphoma, small lymphocytic lymphoma, mantle cell lymphoma), myeloma (e.g., IgM myelomas (e.g., IgM multiple myeloma)), and leukemia (e.g., chronic lymphocytic leukemia (CLL), acute lymphoblastic leukemia, myelogenous leukemia (e.g., chronic myelogenous leukemia, acute myelogenous leukemia (e.g., mast cell leukemia) myeloproliferative diseases (e.g., myelodysplastic syndrome))))).

In certain embodiments, the method described herein is superior (e.g., showing improved safety and/or therapeutic effects; decreased adverse effects) or comparable to existing therapy (e.g., chemotherapy).

In certain embodiments, the biological sample or cell (e.g., the biological sample or cell being contacted with a compound or pharmaceutical composition described herein) is in vitro. In certain embodiments, the cell is in vivo. In certain embodiments, the biological sample or cell is ex vivo.

In certain embodiments, the cell is a malignant cell (e.g., cancer cell). In certain embodiments, the cell is a malignant blood cell. In certain embodiments, the cell is a malignant bone marrow cell. In certain embodiments, the cell is a malignant white blood cell. In certain embodiments, the cell is an adenocarcinoma cell, blastoma cell, carcinoma cell, or sarcoma cell. In certain embodiments, the cell is a pre-malignant cell (e.g., pre-cancerous cell).

In certain embodiments, the method described herein further comprises administering to the subject in need thereof an additional therapy. In certain embodiments, the additional therapy is an additional pharmaceutical agent described herein. In certain embodiments, the additional therapy is a cytotoxic chemotherapy (e.g., gemcitabine, cytarabine, daunorubicin, doxorubicin, vincristine, 1-asparaginase, cyclophosphamide, or etoposide). In certain embodiments, the additional therapy is an epigenetic modifier (e.g., azacitidine or romidepsin). In certain embodiments, the additional therapy is a glucocorticoid. In certain embodiments, the additional therapy is an immunotherapy (e.g., an immunotherapeutic monoclonal antibody). In some embodiments, the additional pharmaceutical agent is etoposide, obatoclax, or navitoclax, and optionally the disease is breast cancer, e.g., triple-negative breast cancer, HER2 positive breast cancer, HER2 negative breast cancer, ER-positive breast cancer, ER-negative breast cancer, or ER/PR-positive breast cancer. In some embodiments, the additional pharmaceutical agent is etoposide, JIB04, or cisplatin. In some embodiments, the additional pharmaceutical agent is JQ1 or NVP2, and optionally the disease is leukemia, e.g., acute myelogenous leukemia, myeloblastic leukemia, promyelocytic leukemia, myelomonocytic leukemia, monocytic leukemia, monoblastic leukemia, or megakaryoblastic leukemia.

In yet another aspect, the present disclosure provides compounds and pharmaceutical compositions described herein for use in the treatment of a disease (e.g., a proliferative disease, such as an IgM gammopathy, mastocytosis, or cancer) in a subject in need thereof.

In yet another aspect, the present disclosure provides compounds and pharmaceutical compositions described herein for use in the prevention of a disease (e.g., a proliferative disease, such as an IgM gammopathy, mastocytosis, or cancer) in a subject in need thereof.

In another aspect, the present disclosure provides compounds and pharmaceutical compositions described herein for use in inhibiting the activity of a kinase (e.g., SRC Family kinases (e.g., HCK, LYN), Tec family kinases (e.g., BTK)) in a subject in need thereof.

In another aspect, the present disclosure provides compounds and pharmaceutical compositions described herein for use in inhibiting the activity of a kinase in a biological sample (e.g., an in vivo or ex vivo biological sample).

In another aspect, the present disclosure provides compounds and pharmaceutical compositions described herein for use in inhibiting the activity of a kinase in a cell (e.g., an in vivo or ex vivo cell).

In another aspect, the present disclosure provides uses of compounds and pharmaceutical compositions described herein in the manufacture of a medicament for treating a disease in a subject in need thereof.

In another aspect, the present disclosure provides uses of compounds and pharmaceutical compositions described herein in the manufacture of a medicament for preventing a disease in a subject in need thereof.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Example 1. In-Vivo Rat Efficacy Study in MYD88 Mutated ABC DLBCL TMD8 Xenografted Rats

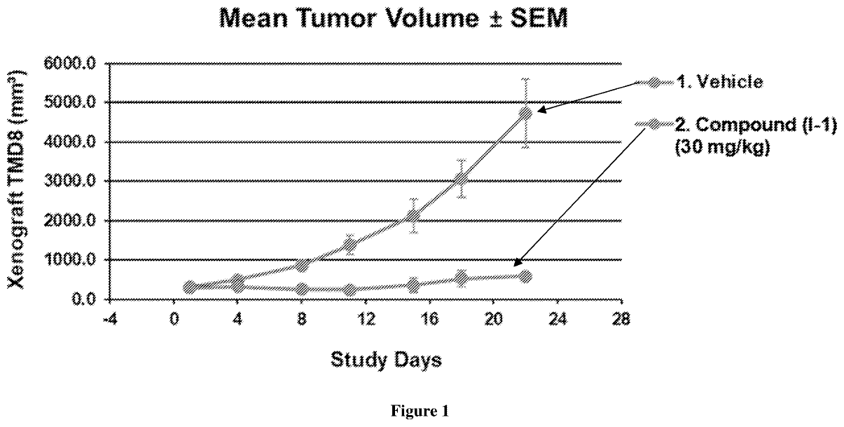

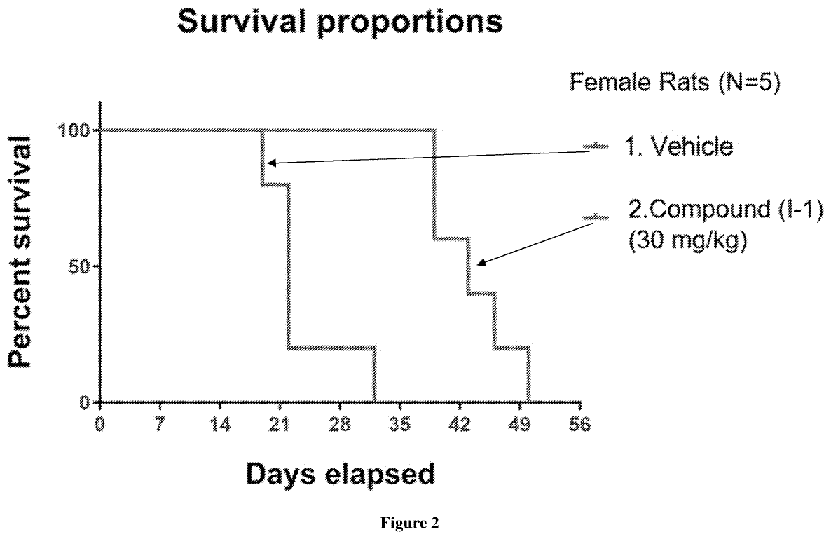

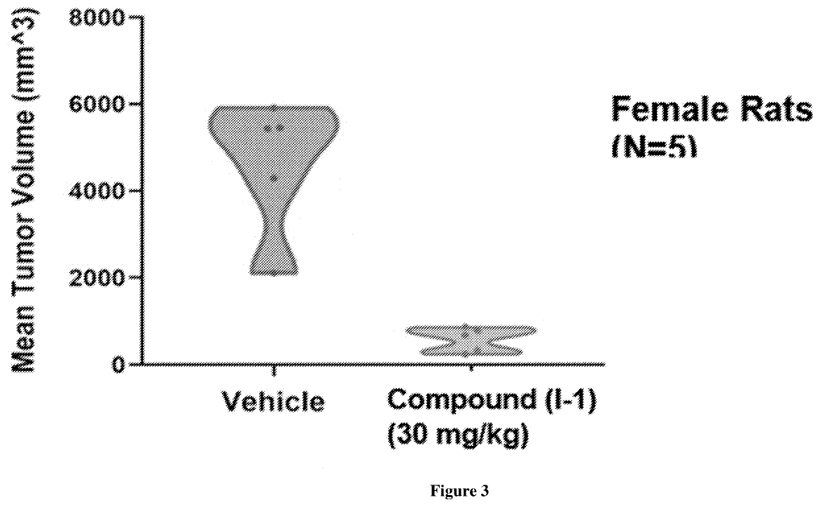

A rat efficacy study for was carried out with Compound (I-1) using an MYD88 mutated ABC DLBCL TMD8 xenografted rat model. Pharmacodynamic studies in mice show inhibition of HCK, BTK, and NFκB, and dose-related tumor redistribution. Administration of Compound (I-1) at 30 mg/kg resulted in lower Mean tumor volume compared to vehicle (FIG. 1 and FIG. 3). Survival proportions were measured, and mice treated with Compound (I-1) had a 43 survival days, compared to 22 survival days in vehicle (FIG. 2).

It was found that Compound (I-1) inhibits HCK in a dose dependent Type II inhibition in MYD88 mutated WM and ABC DLBCL cell lines and primary WM cells. Compound (I-1) also exhibits potent apoptotic effects (vs. ibrutinib or A419259) in MYD88 mutated WM and ABC DLBCL cell lines, primary WM cells, and sparing of healthy donor B- and T-cells.

### Example 2. In-Vitro Assays

In-vitro assays were carried out to test the activity of the compounds against different kinases and cell lines.

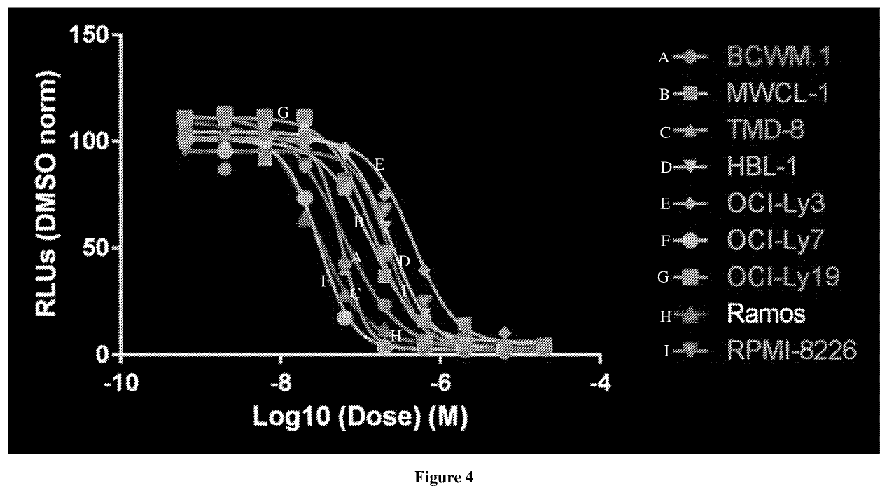

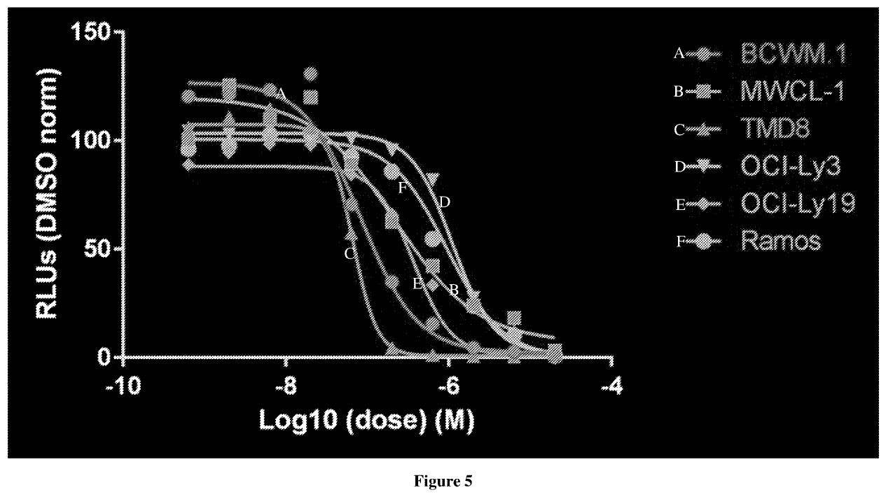

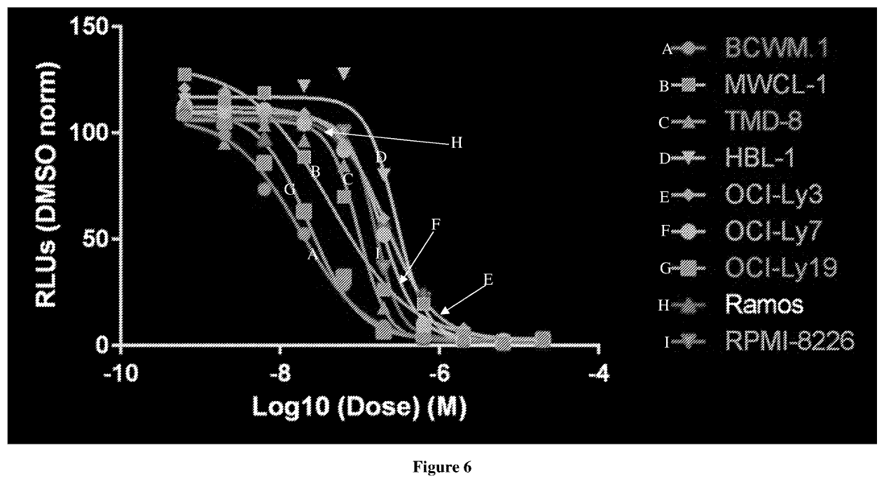

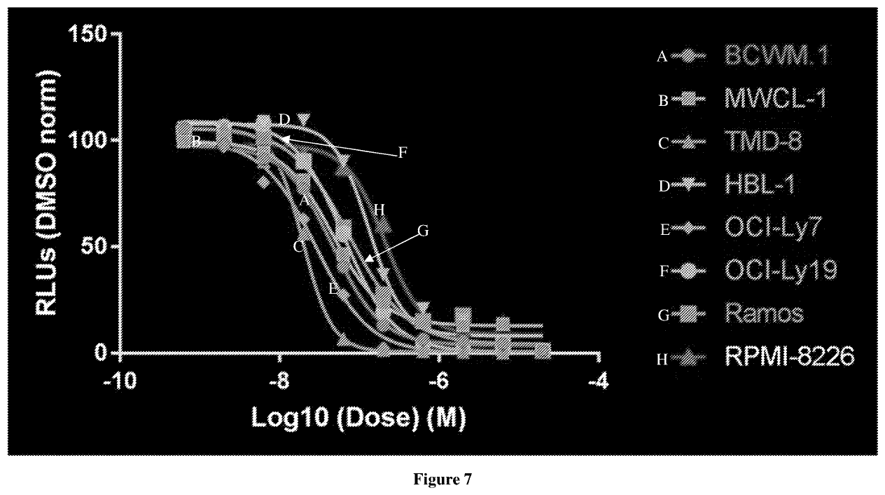

The compounds were tested against several different cell lines, and the ED₅₀ values determined, along with the microsome stability. See Table 1 and Table 2. The plots of this data are shown for Compound (I-1) in FIG. 4 (indicated with labels A-I), Compound (I-12) in FIG. 5 (indicated with labels A-F), Compound (I-4) in FIG. 6 (indicated with labels A-I), and Compound (I-16) in FIG. 7 (indicated with labels A-H).

TABLE 1
ED₅₀ values for compounds across different cell lines

| Compound | BCWM.1 | MWCL-1 | TMD8 | HBL-1 | OCI-Ly3 |
|---|---|---|---|---|---|
| (I-1) | 5.88E−08 | 1.33E−07 | 5.74E−08 | 2.36E−07 | 4.51E−07 |
| (I-12) | 8.20E−08 | 1.20E−07 | 6.91E−08 | | 6.16E−07 |
| (I-13) | 1.25E−07 | 2.47E−07 | 1.68E−07 | | 6.28E−07 |
| (I-14) | 1.55E−07 | 9.04E−08 | 3.00E−08 | | 1.01E−06 |
| (I-2) | 3.87E−08 | 1.01E−07 | 5.93E−08 | | 1.77E−07 |
| (I-3) | 2.15E−07 | 5.42E−07 | 2.05E−07 | | 8.15E−07 |
| (I-4) | 1.77E−08 | 4.27E−08 | 9.63E−08 | 2.86E−07 | 2.09E−07 |
| (I-5) | 1.60E−06 | 2.57E−06 | 1.67E−06 | 6.37E−06 | |
| (I-6) | 6.38E−07 | 1.17E−06 | 6.31E−07 | 1.24E−06 | |
| (I-7) | 1.32E−06 | 1.78E−06 | 1.11E−06 | 4.09E−06 | |
| (I-8) | 7.25E−07 | 1.28E−06 | 1.27E−06 | 2.47E−06 | |
| (I-15) | 1.05E−07 | 1.40E−07 | 2.26E−08 | 2.61E−07 | |
| (I-16) | 4.31E−08 | 4.56E−08 | 1.92E−08 | 1.34E−07 | |
| (I-9) | 4.66E−07 | 8.35E−07 | 6.54E−07 | 2.79E−06 | |
| (I-10) | 2.00E−06 | 3.88E−06 | 1.09E−06 | 1.05E−05 | |
| (I-11) | 4.09E−07 | 1.40E−06 | 6.29E−07 | 2.84E−06 | |
| (I-19) | 3.39E−07 | 7.10E−07 | 2.96E−07 | 2.98E−06 | |
| (I-21) | 2.85E−07 | 3.44E−07 | 1.95E−07 | 8.50E−07 | |
| (I-22) | 1.15E−07 | 2.56E−07 | 3.68E−08 | 5.35E−07 | |
| (I-23) | 3.95E−07 | 2.27E−07 | 1.13E−06 | 9.73E−07 | |
| (I-24) | 2.87E−07 | 1.38E−06 | 1.67E−06 | 6.18E−05 | |
| TL2-059 | 1.22E−09 | 3.93E−07 | 1.52E−07 | | 1.49E−06 |

TABLE 2
ED₅₀ values for compound across different cell lines

| | | | | | microsome |
|---|---|---|---|---|---|
| Compound | OCI-Ly7 | OCI-Ly19 | Ramos | RPMI-8226 | stability (T_{1/2}, min) |
| (I-1) | 3.02E−08 | 1.44E−07 | 2.90E−08 | 2.23E−07 | 42.4 (mouse); 60.2 |
| | | | | | (rat); 79.3 (human) |
| (I-12) | 1.11E−07 | 2.71E−07 | 5.61E−07 | 7.53E−07 | 22.9 (mouse) |
| (I-13) | 2.86E−07 | 3.63E−07 | 8.83E−07 | 1.04E−06 | |
| (I-14) | 3.03E−08 | 1.30E−07 | 8.67E−08 | 3.86E−07 | 20.4 (mouse) |
| (I-2) | 6.12E−08 | 1.59E−07 | 1.09E−07 | 3.92E−07 | 41.7 (mouse) |
| (I-3) | 2.34E−07 | 1.86E−07 | 2.83E−07 | 5.31E−07 | |
| (I-4) | 1.84E−07 | 2.35E−08 | 2.26E−07 | 1.55E−07 | 5.7 (mouse) |
| (I-5) | 4.07E−06 | 1.59E−06 | 3.58E−06 | 6.46E−06 | |
| (I-6) | 8.42E−07 | 6.82E−07 | 1.22E−06 | 1.45E−06 | |
| (I-7) | 3.05E−06 | 1.66E−06 | 6.40E−06 | 6.35E−06 | |
| (I-8) | 1.79E−06 | 9.23E−07 | 2.33E−06 | 3.96E−06 | |
| (I-15) | 1.26E−07 | 1.22E−07 | 7.23E−07 | 5.72E−07 | |
| (I-16) | 2.86E−08 | 6.40E−08 | 7.55E−08 | 2.06E−07 | 8.6 (mouse) |
| (I-9) | 8.83E−07 | 8.44E−07 | 1.16E−06 | 1.36E−06 | |
| (I-10) | 2.66E−06 | 6.40E−07 | 4.42E−06 | 1.14E−05 | |
| (I-11) | 1.49E−06 | 6.80E−07 | 1.48E−06 | 2.53E−06 | |
| (I-19) | 5.99E−07 | 9.07E−07 | 4.90E−07 | 2.70E−06 | |
| (I-21) | 7.67E−08 | 3.84E−07 | 7.75E−08 | 4.89E−07 | |
| (I-22) | 5.26E−08 | 2.98E−07 | 6.11E−08 | 3.90E−07 | |
| (I-23) | 1.39E−07 | 1.04E−06 | 1.55E−07 | 3.23E−06 | |
| (I-24) | 1.55E−07 | 4.85E−07 | 2.15E−07 | | |
| TL2-059 | 1.35E−07 | 3.77E−07 | 2.92E−07 | | 52 (mouse); 61.4 |
| | | | | | (rat); >120 (human) |

The IC₅₀ values were also determined for Compound (I-1) using an Invitrogen kinase assay. The results are shown in Table 2, along with comparison data with TL2-059.

TABLE 2
IC₅₀ values from Invitrogen kinase assay
of compounds with different kinases.

| Kinase | Compound (I-1) | TL2059 |
|---|---|---|
| MAP4K2 (GCK) | 8.35 | 30.5 |
| LYN A | 8.77 | 23.8 |
| MAPK14 (p38 alpha) | 79.7 | 164 |
| Direct | | |
| FER | 9.81 | 36.1 |
| MAP4K1 (HPK1) | 11.8 | 55 |
| FES (FPS) | 11 | 50.8 |
| ABL1 | 32.3 | 62.6 |
| ABL2 (Arg) | 45.5 | 73.6 |
| MAP3K7/MAP3K7IP1 | 24.1 | 129 |
| (TAK1-TAB1) | | |
| SRC | 77.5 | 117 |
| LCK | 4.73 | 13.4 |
| HCK | 7.39 | 16.6 |
| TAOK3 (JIK) | 413 | 734 |
| FGR | 8.02 | Not determined |
| TAOK2 (TAO1) | 61.3 | 238 |
| FYN | 419 | 619 |
| TAOK1 | 194 | 314 |

### Example 3. Synthesis of Compounds

Unless otherwise noted, reagents and solvents were used as received from commercial suppliers. Proton nuclear magnetic resonance spectra were obtained on Bruker AVANCE spectrometer at 400 MHz for proton. Spectra are given in ppm (d) and coupling constants, J, are reported in Hertz. The solvent peak was used as the reference peak for proton spectra. LC-MS spectra were obtained on Agilent 1100 HPLC LC-MS ion trap electrospray ionization (ESI) mass spectrometer.

### Compound (I-12)

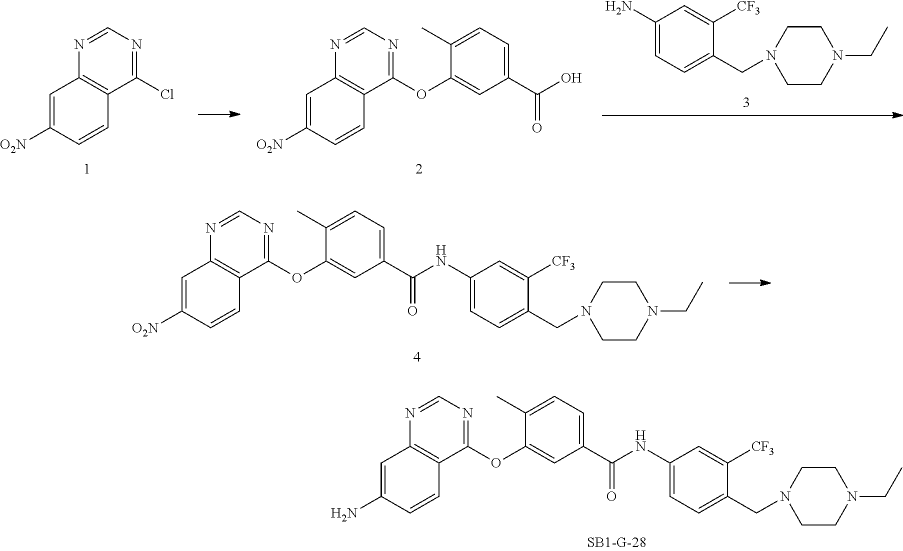 [SMILES: O=[N+]([O-])c1ccc2c(Cl)ncnc2c1 ; Cc1ccc(C(=O)O)cc1Oc1ncnc2cc([N+](=O)[O-])ccc12 ; CCN1CCN(Cc2ccc(N)cc2C(F)(F)F)CC1 ; CCN1CCN(Cc2ccc(NC(=O)c3ccc(C)c(Oc4ncnc5cc([N+](=O)[O-])ccc45)c3)cc2C(F)(F)F)CC1 ; CCN1CCN(Cc2ccc(NC(=O)c3ccc(C)c(Oc4ncnc5cc(N)ccc45)c3)cc2C(F)(F)F)CC1]

### 4-methyl-3-(7-nitroquinazolin-4-yloxy)benzoic Acid (2)

The mixture of 1 (880 mg, 4.20 mmol), 2 (640 mg, 4.21 mmol), Cs₂CO₃ (2.8 g, 8.59 mmol) in i-PrOH (50 mL) was stirred at 50° C. overnight, after the reaction was completed the mixture was concentrated to remove the solvent, added H₂O (50 mL), then added con HCl to make PH<7, filtered, washed with H₂O, dried to get 3 (light yellow solid, 1.1 g, yield 78%). LCMS (m/z): 326 [M+H]⁺.

### N-(4-((4-ethylpiperazin-1-yl)methyl)-3-(trifluoromethyl)phenyl)-4-methyl-3-(7-nitroquinazolin-4-yloxy)benzamide (4)

To the mixture of 2 (1.0 g, 3.07 mmol) in DMF (0.3 mL) and DCM (30 mL) was added (COCl)₂ (5 mL, excess) dropwise under 0° C., stirred at 0° C. for 1 h, then concentrated to remove the solvent, the residue was re-dissolved in DCM (20 mL), followed by addition of 3 (900 mg, 3.13 mmol) in DIPEA (3 mL) and DCM (10 mL) under 0° C., further stirred at 0° C. to r.t for 2 h, after the reaction was completed the mixture was concentrated to remove the solvent, then extracted with ethyl acetate (80 mL×3), the organic phase was washed with brine (50 mL×2), dried over Na₂SO₄, filtered, concentrated and purified by silica gel (DCM/MeOH=8/1) to obtain 4 (light brown solid, 450 mg, yield 25%). LCMS (m/z): 595 [M+H]⁺.

### 3-(7-aminoquinazolin-4-yloxy)-N-(4-((4-ethylpiperazin-1-yl)methyl)-3-(trifluoromethyl)phenyl)-4-methylbenzamide (Compound (I-12))

The mixture of 4 (70 mg, 0.118 mmol), Pd/C (20 mg) in MeOH (10 mL) was stirred at r.t under H₂ (1 atm) overnight, filtered, concentrated to remove the solvent, purified by prep-TLC (DCM/MeOH=8/1) to get Compound (I-12) (light yellow solid, 50 mg, 76%). LCMS (m/z): 565 [M+H]⁺. ¹H NMR (400 MHz, DMSO-d₆) δ 10.44 (s, 1H), 8.37 (s, 1H), 8.18 (d, J=2.0 Hz, 1H), 8.07 (d, J=8.0 Hz, 1H), 8.04 (d, J₁=10.0 Hz, J₂=2.0 Hz, 1H), 7.87 (d, J=7.6 Hz, 1H), 7.85 (s, 1H), 7.69 (d, J=8.8 Hz, 1H), 7.53 (d, J=8.4 Hz, 1H), 7.08 (dd, J₁=8.8 Hz, J₂=2.4 Hz, 1H), 6.85 (d, J=2.0 Hz, 1H), 6.41 (s, 2H), 3.55 (s, 2H), 2.27-2.38 (m+q, 8+2 H), 2.17 (s, 3H), 0.98 (t, J=9.6 Hz, 3H).

### Compound (I-13)

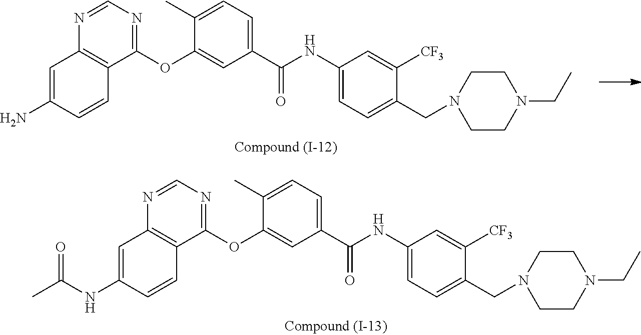

### 3-(7-acetamidoquinazolin-4-yloxy)-N-(4-((4-ethylpiperazin-1-yl)methyl)-3-(trifluoromethyl)phenyl)-4-methylbenzamide (Compound (I-13))

To the mixture of Compound (I-12) (50 mg, 0.0886 mmol), DIPEA (1 mL) in CH₃CN (5 mL) was added Acetyl chloride (15 mg, 0.191 mmol), then stirred at r.t for 4 h, concentrated to remove the solvent the residue was purified by prep-TLC (DCM/MeOH=10/1) and prep-HPLC (C18 column, CH₃CN/H₂O, containing 0.05% NH₄HCO₃) to obtain Compound (I-13) (light yellow solid, 6 mg, yield 11%). LCMS (m/z): 607 [M+H]⁺. ¹H NMR (400 MHz, DMSO-d₆) δ 8.60 (s, 1H), 8.45 (s, 1H), 8.44 (d, J=7.2 Hz, 1H), 8.14 (d, J=2.0 Hz, 1H), 7.89-7.95 (m, 3H), 7.84 (d, J=1.6 Hz, 1H), 7.77 (d, J=8.4 Hz, 1H), 7.55 (d, J=7.6 Hz, 1H), 4.62 (s, 2H), 3.68 (s, 2H), 2.47-2.58 (m, 10H), 2.27 (s, 3H), 2.26 (s, 3H), 1.13 (t, J=7.2 Hz, 3H).

### Compound (I-14)

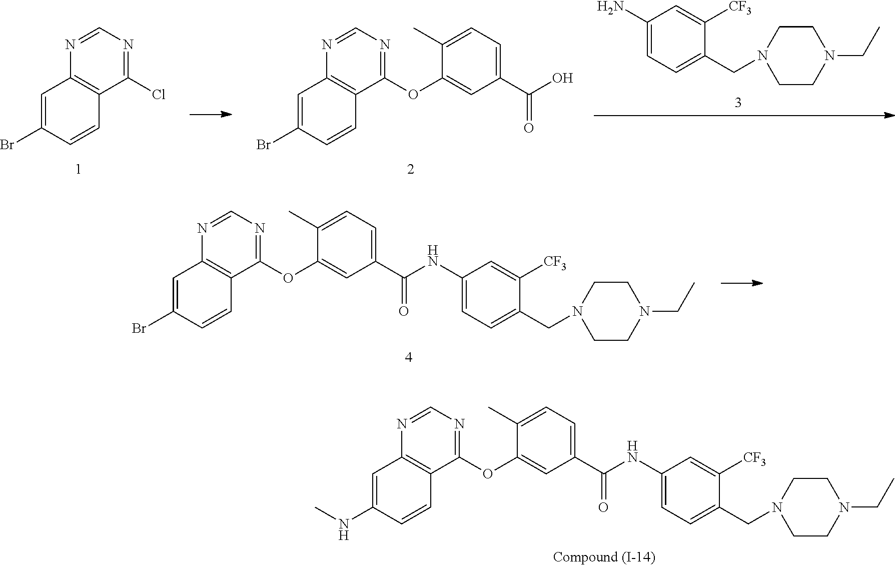

### 3-(7-bromoquinazolin-4-yloxy)-4-methylbenzoic Acid (2)

The mixture of 1 (550 mg, 2.26 mmol), 2 (352 mg, 2.31 mmol), Cs₂CO₃ (900 mg, 2.76 mmol) in i-PrOH (30 mL) was stirred at 50° C. overnight, after the reaction was completed the mixture was concentrated to remove the solvent, added H₂O (50 mL), then added con HCl to make PH<7, filtered, washed with H₂O, dried to get 2 (white solid, 800 mg, yield 83%). LCMS (m/z): 360 [M+H]⁺.

### 3-(7-bromoquinazolin-4-yloxy)-N-(4-((4-ethylpiperazin-1-yl)methyl)-3-(trifluoromethyl)phenyl)-4-methylbenzamide (4)

To the mixture of 2 (510 mg, 1.42 mmol) in DMF (0.2 mL) and DCM (25 mL) was added (COCl)₂ (3 mL, excess) dropwise under 0° C., stirred at 0° C. for 1H, then concentrated to remove the solvent, the residue was re-dissolved in DCM (30 mL), followed by addition of 3 (500 mg, 1.74 mmol) in DIPEA (3 mL) and DCM (10 mL) under 0° C., further stirred at 0° C. to r.t for 3 h, after the reaction was completed the mixture was concentrated to remove the solvent, then extracted with ethyl acetate (100 mL), the organic phase was washed with brine (50 mL×2), dried over Na₂SO₄, filtered, concentrated and purified by silica gel (DCM/MeOH=20/1) to obtain 4 (light yellow solid, 500 mg, yield 56%). LCMS (m/z): 628 [M+H]⁺.

### N-(4-((4-ethylpiperazin-1-yl)methyl)-3-(trifluoromethyl)phenyl)-4-methyl-3-(7-(methylamino)quinazolin-4-yloxy)benzamide (Compound (I-14))

The mixture of 4 (120 mg, 0.191 mmol) and MeNH₂—HCl (80 mg, 1.18 mmol), Pd₂(dba)₃ (20 mg, 0.0218 mmol), XantPhos (25 mg, 0.0432 mmol), Cs₂CO₃ (530 mg, 1.63 mmol) in dioxane (3 mL) was stirred at 130° C. for 30 min under N₂ in microwave, after completion, filtered, concentrated under reduced pressure, the residue was extracted with ethyl acetate (30 mL×3), the organic phase was washed with brine (20 mL×2), dried over Na₂SO₄, filtered, concentrated and purified by prep-TLC (DCM/MeOH=9/1) and prep-HPLC (C18 column, CH₃CN/H₂O, containing 0.05% NH₄HCO₃) to obtain Compound (I-14) (white solid, 28 mg, yield 25%). LCMS (m/z): 579 [M+H]⁺. ¹H NMR (400 MHz, DMSO-d₆) δ 10.44 (s, 1H), 8.41 (s, 1H), 8.18 (d, J=2.0 Hz, 1H), 8.07 (d, J=8.8 Hz, 1H), 8.04 (d, J=8.8 Hz, 1H), 7.88 (dd, J₁=8.8 Hz, J₂=1.6 Hz, 1H), 7.86 (d, J=2.8 Hz, 1H), 7.69 (d, J=8.8 Hz, 1H), 7.53 (d, J=7.6 Hz, 1H), 7.11 (dd, J₁=9.2 Hz, J₂=2.4 Hz, 1H), 7.00 (d, J=5.2 Hz, 1H), 6.69 (d, J=2.0 Hz, 1H), 3.55 9 s, 2H), 2.84 (d, J=4.8 Hz, 3H), 2.27-2.38 (m, 10H), 2.17 (s, 3H), 2.17 (s, 3H), 0.97 (t, J=7.2 Hz, 3H).

### Compound (I-2)

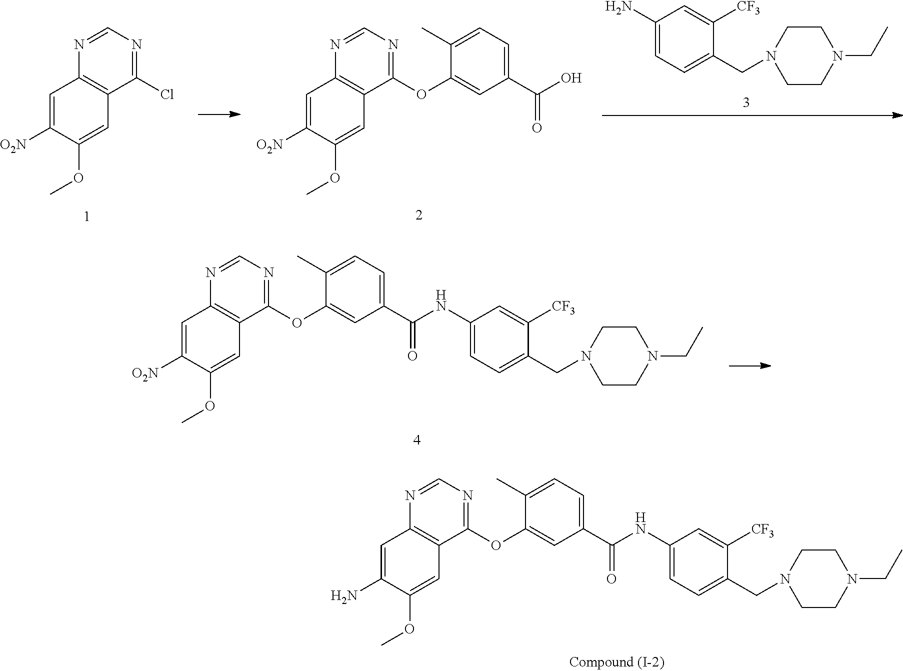 [SMILES: COc1cc2c(Cl)ncnc2cc1[N+](=O)[O-] ; COc1cc2c(Oc3cc(C(=O)O)ccc3C)ncnc2cc1[N+](=O)[O-] ; CCN1CCN(Cc2ccc(N)cc2C(F)(F)F)CC1 ; CCN1CCN(Cc2ccc(NC(=O)c3ccc(C)c(Oc4ncnc5cc([N+](=O)[O-])c(OC)cc45)c3)cc2C(F)(F)F)CC1 ; CCN1CCN(Cc2ccc(NC(=O)c3ccc(C)c(Oc4ncnc5cc(N)c(OC)cc45)c3)cc2C(F)(F)F)CC1]

### 3-(7-amino-6-methoxyquinazolin-4-yloxy)-N-(4-((4-ethylpiperazin-1-yl)methyl)-3-(trifluoromethyl)phenyl)-4-methylbenzamide (Compound (I-2))

The mixture of 4 (400 mg, 0.640 mmol), Pd/C (80 mg) in MeOH (10 mL) was stirred at r.t under H₂ (1 atm) overnight, filtered, concentrated to remove the solvent, purified by flash column (DCM/MeOH=25/1) to get Compound (I-2) (light yellow solid, 380 mg, 44%). LCMS (m/z): 595 [M+H]⁺. ¹H NMR (400 MHz, DMSO-d₆) δ 10.45 (s, 1H), 8.34 (s, 1H), 8.18 (d, J=2.0 Hz, 1H), 8.05 (d, J=8.8 Hz, 1H), 7.88 (d, J₁=9.2 Hz, J₂=1.2 Hz, 1H), 7.86 (s, 1H), 7.70 (d, J=8.4 Hz, 1H), 7.54 (d, J=8.0 Hz, 1H), 7.46 (s, 1H), 6.98 (s, 2H), 6.22 (s, 1H), 4.01 (s, 3H), 3.55 (s, 2H), 2.27-2.38 (m+q, 8+2 H), 2.18 (s, 3H), 0.98 (t, J=9.6 Hz, 3H).

### Compound (I-3)

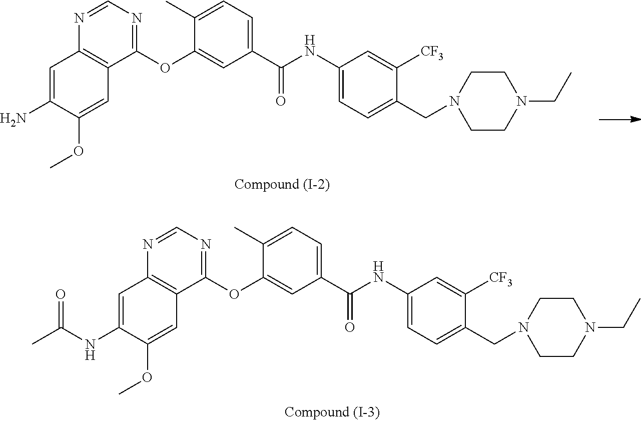

### 3-(7-acetamido-6-methoxyquinazolin-4-yloxy)-N-(4-((4-ethylpiperazin-1-yl)methyl)-3-(trifluoromethyl)phenyl)-4-methylbenzamide (Compound (I-3))

To the mixture of Compound (I-2) (80 mg, 0.135 mmol), DIPEA (0.5 mL) in CH₃CN (6 mL) was added Acetyl chloride (40 mg, 0.509 mmol), then stirred at r.t overnight, concentrated to remove the solvent the residue was purified by prep-TLC (DCM/NH₃-MeOH=25/1) and prep-HPLC (C18 column, CH₃CN/H₂O, containing 0.05% NH₄HCO₃) to obtain Compound (I-3) (white solid, 4 mg, yield 5%). LCMS (m/z): 637 [M+H]⁺. ¹H NMR (400 MHz, MeOD) δ 8.83 (s, 1H), 8.40 (s, 1H), 8.02 (d, J=2.4 Hz, 1H), 7.76-7.81 (m, 2H), 7.72 (d, J=2.0 Hz, 1H), 7.67 (s, 2H), 7.43 (d, J=8.0 Hz, 1H), 4.04 (s, 2H), 3.55 (s, 2H), 2.35-2.46 (m, 10H), 2.20 (s, 3H), 2.16 (s, 3H), 1.01 (t, J=7.2 Hz, 3H).

### Compound (I-1)

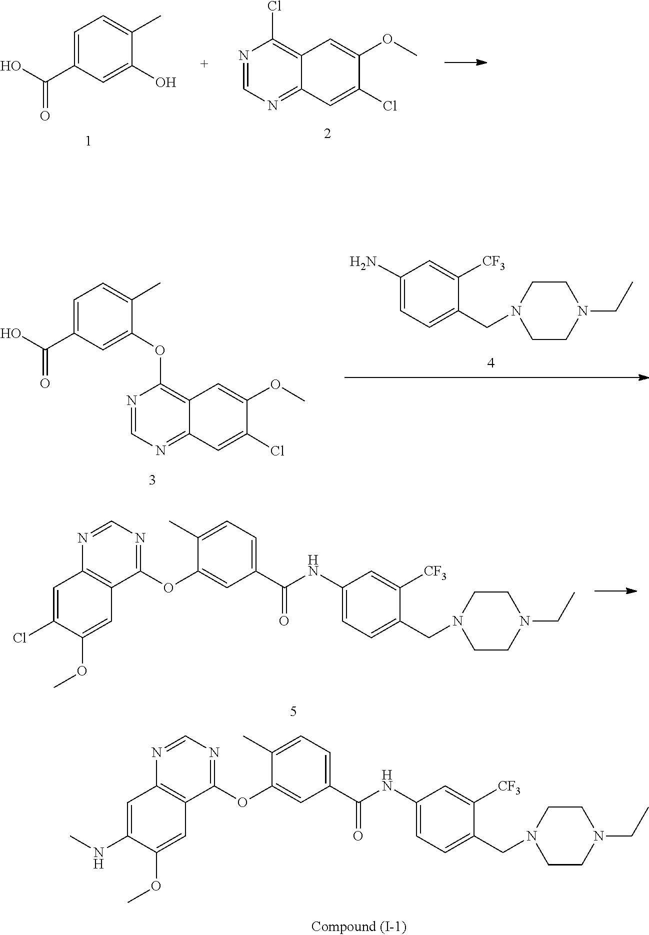

### 3-(7-chloro-6-methoxyquinazolin-4-yloxy)-4-methylbenzoic Acid (3)

The mixture of 1 (5.0 g, 21.8 mmol), 2 (3.32 g, 21.8 mmol), Cs₂CO₃ (14.5 g, 44.5 mmol) in i-PrOH (70 mL) was stirred at 50° C. overnight, after the reaction was completed the mixture was concentrated to remove the solvent, added H₂O (200 mL), then added con HCl to make PH<7, filtered, washed with H₂O, dried to get 3 (white solid, 7.6 g, yield 98%). LCMS (m/z): 345 [M+H]⁺.

### 3-(7-chloro-6-methoxyquinazolin-4-yloxy)-N-(4-((4-ethylpiperazin-1-yl)methyl)-3-(trifluoromethyl)phenyl)-4-methylbenzamide (5)

To the mixture of 3 (25 g, 72.5 mmol) in DMF (2 mL) and DCM (500 mL) was added (COCl)₂ (50 mL, excess) dropwise under 0° C., stirred at 0° C. for 1H, then concentrated to remove the solvent, the residue was re-dissolved in DCM (200 mL), followed by addition of 4 (21 g, 73.1 mmol) in DIPEA (100 mL) and DCM (400 mL) under 0° C., further stirred at 0° C. to r.t overnight, after the reaction was completed the mixture was concentrated to remove the solvent, then extracted with ethyl acetate (2 L×4), the organic phase was washed with brine (1 L×2), dried over Na₂SO₄, filtered, concentrated and purified by silica gel (DCM/MeOH=50/1) to obtain 5 (white solid, 26 g, yield 58%). LCMS (m/z): 614 [M+H]⁺.

### N-(4-((4-ethylpiperazin-1-yl)methyl)-3-(trifluoromethyl)phenyl)-3-(6-methoxy-7-(methylamino)quinazolin-4-yloxy)-4-methylbenzamide (Compound (I-1))

The mixture of 5 (1.3 g, 2.12 mmol) and MeNH₂—HCl (1.43 g, 21.2 mmol), Pd₂(dba)₃ (195 mg, 0.213 mmol), JohnPhos (127 mg, 0.427 mmol), t-BuONa (2.44 g, 25.4 mmol) in toluene (20 mL) was stirred at r.t overnight under N₂ in sealed tube, after completion, diluted with EA, acidified with AcOH, filtered, washed with EA, the organic phase was concentrated to remove the solvent, the residue was purified by flash column (DCM/MeOH=50/1-40/1) and prep-HPLC (C18 column, CH₃CN/H₂O, containing 0.05% NH₄HCO₃) to obtain Compound (I-1) (light yellow solid, 374 mg, yield 29%). LCMS (m/z): 609 [M+H]⁺. ¹H NMR (400 MHz, DMSO-d₆) δ 10.45 (s, 1H), 8.37 (s, 1H), 8.18 (d, J=2.0 Hz, 1H), 8.05 (d, J=8.4 Hz, 1H), 7.88 (dd, J1=8.8 Hz, J2=1.6 Hz, 1H), 7.87 (d, J=1.6 Hz, 1H), 7.69 (d, J=8.4 Hz, 1H), 7.54 (d, J=8.0 Hz, 1H), 7.44 (s, 1H), 6.72 (s, 1H), 6.57 (d, J=5.2 Hz, 1H), 4.02 (s, 3H), 3.55 (s, 2H), 2.88 (d, J=5.2 Hz, 3H), 2.27-2.38 (m, 10H), 2.17 (s, 3H), 2.18 (s, 3H), 0.97 (t, J=7.2 Hz, 3H).

### Compound (I-4)

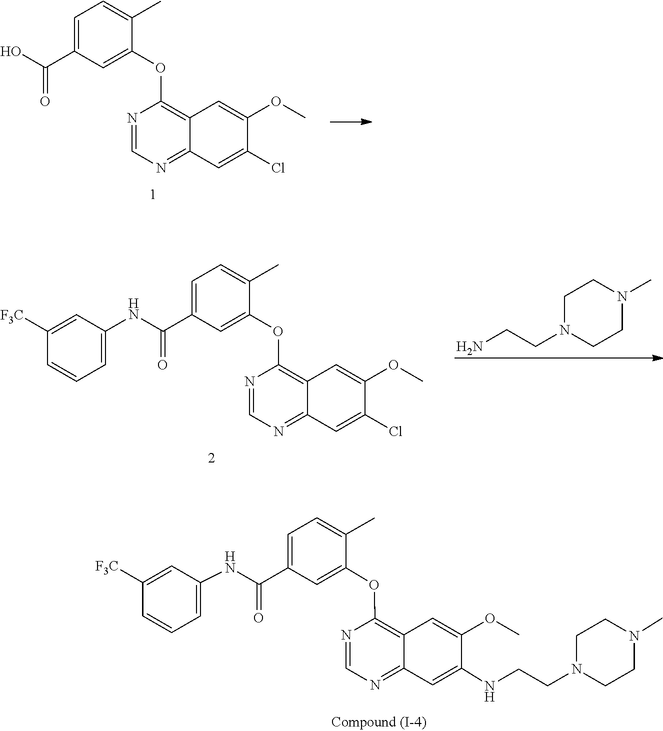

### 3-(7-chloro-6-methoxyquinazolin-4-yloxy)-4-methyl-N-(3-(trifluoromethyl)phenyl)benzamide (2)

To a solution of 3-(7-chloro-6-methoxyquinazolin-4-yloxy)-4-methylbenzoic acid (500 mg, 1.45 mmol) in DCM (100 mL) was added (COCl)₂ (365 mg, 2.9 mmol) and a drop of DMF at 0° C., stirred at 0° C. for 2 h. The reaction mixture was added to the solution of 3-(trifluoromethyl)aniline (234 mg, 1.45 mmol) and DIPEA (561 mg, 4.35 mmol) in DCM (100 mL) at 0° C., stirred at 0° C. for 2 h. after the reaction was completed the mixture was concentrated and purified by silica gel (DCM/MeOH=10/1) to obtain 2 (yellow solid, 500 mg, 70% yield). LCMS: 488 (M+H)⁺.

### 3-(6-methoxy-7-(2-(4-methylpiperazin-1-yl)ethylamino)quinazolin-4-yloxy)-4-methyl-N-(3-(trifluoromethyl)phenyl)benzamide (Compound (I-4))

The mixture of 4 (76 mg, 0.156 mmol), SM (55 mg, 0.384 mmol), CS₂CO₃ (110 mg, 0.338 mmol), Xant-phos (19 mg, 0.0328 mmol), and Pd₂(dba)₃ (15 mg, 0.0164 mmol) in DMSO (2 mL) was stirred at 150° C. for 4 h, after completion, extracted with ethyl acetate (30 mL×3), the organic phase was washed with brine (20 mL×2), dried with Na₂SO₄, filtered, removed the solvent, the residue was purified by prep-TLC (DCM/MeOH=10/1) and prep-TLC (DCM/MeOH—NH₃=15/1) to obtain Compound (I-4) (off-white solid, 6 mg, yield 7%). LCMS (m/z): 595 [M+H]⁺; ¹H NMR (400 MHz, DMSO) δ 10.50 (s, 1H), 8.38 (s, 1H), 8.22 (s, 1H), 8.06 (d, J=8.8 Hz, 1H), 7.90 (d, J=8.0 Hz, 1H), 7.87 (s, 1H), 7.59 (t, J=8.0 Hz, 1H), 7.54 (d, J=8.0 Hz, 1H), 7.46 (s, 1H), 7.45 (d, J=8.8 Hz, 1H), 6.82 (s, 1H), 6.19 (t, J=5.2 Hz, 1H), 4.03 (s, 3H), 3.36 (t, J=6.0 Hz, 2H), 2.62 (t, J=6.0 Hz, 2H), 2.34-2.42 (m, 8H), 2.18 (s, 3H), 2.17 (s, 3H).

### Compound (I-5)

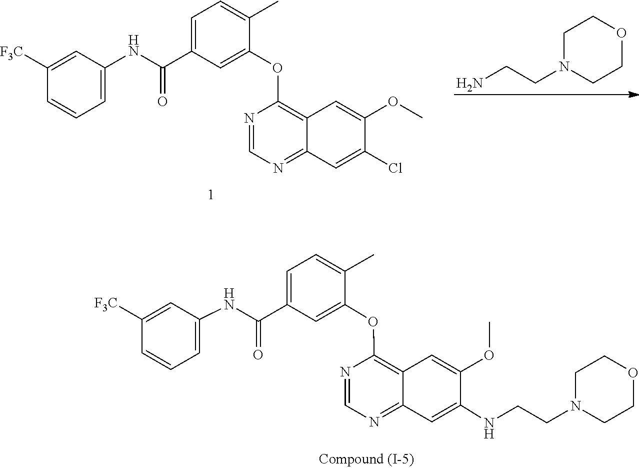 [SMILES: COc1cc2c(Oc3cc(C(=O)Nc4cccc(C(F)(F)F)c4)ccc3C)ncnc2cc1Cl ; NCCN1CCOCC1 ; COc1cc2c(Oc3cc(C(=O)Nc4cccc(C(F)(F)F)c4)ccc3C)ncnc2cc1NCCN1CCOCC1]

### 3-(6-methoxy-7-(2-morpholinoethylamino)quinazolin-4-yloxy)-4-methyl-N-(3-(trifluoromethyl)phenyl)benzamide (Compound (I-5))

The mixture of 3-(7-chloro-6-methoxyquinazolin-4-yloxy)-4-methyl-N-(3-(trifluoromethyl)phenyl)benzamide (120 mg, 0.24 mmol), 2-morpholineethanamine (31 mg, 0.24 mmol), Cs₂CO₃ (156 mg, 0.48 mmol), Xant-phos (14 mg, 0.024 mmol), and Pd₂(dba)₃ (24 mg, 0.028 mmol) in toluene (5 mL) was stirred at 100° C. for 16 h, after the reaction was completed the mixture was concentrated and purified by prep-HPLC (C18 column, CH₃CN/H₂O, containing 0.05% NH₄HCO₃) to get Compound (I-5) (white solid, 76 mg, 51%). LCMS: 582 (M+H)+; HPLC: 100% (@254 nm); ¹H NMR (400 MHz, DMSO) δ 10.51 (s, 1H), 8.38 (s, 1H), 8.23 (s, 1H), 8.07 (d, J=8.3 Hz, 1H), 7.95-7.81 (m, 2H), 7.62-7.35 (m, 4H), 6.83 (s, 1H), 6.23 (t, J=5.2 Hz, 1H), 4.04 (s, 3H), 3.70-3.51 (m, 4H), 3.38 (dd, J=11.8, 6.1 Hz, 2H), 2.62 (t, J=6.4 Hz, 2H), 2.40 (d, J=47.2 Hz, 4H), 2.18 (s, 3H).

### Compound (I-6)

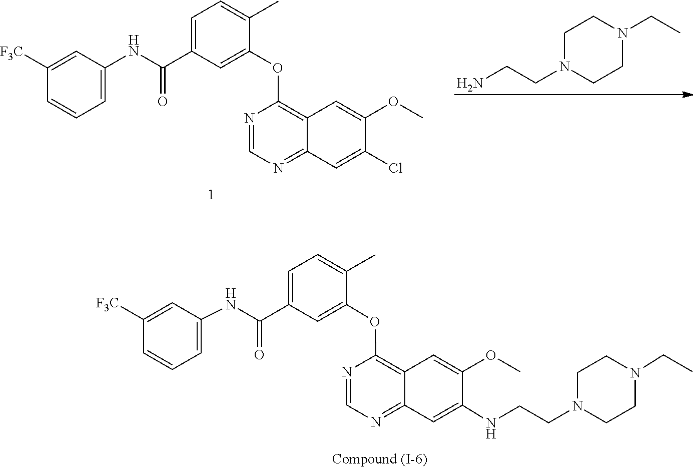 [SMILES: COc1cc2c(Oc3cc(C(=O)Nc4cccc(C(F)(F)F)c4)ccc3C)ncnc2cc1Cl ; CCN1CCN(CCN)CC1 ; CCN1CCN(CCNc2cc3ncnc(Oc4cc(C(=O)Nc5cccc(C(F)(F)F)c5)ccc4C)c3cc2OC)CC1]

### 3-(7-(2-(4-ethylpiperazin-1-yl)ethylamino)-6-methoxyquinazolin-4-yloxy)-4-methyl-N-(3-(trifluoromethyl)phenyl)benzamide (Compound (I-6))

The mixture of 3-(7-chloro-6-methoxyquinazolin-4-yloxy)-4-methyl-N-(3-(trifluoromethyl)phenyl)benzamide (120 mg, 0.24 mmol), 2-(4-ethylpiperazin-1-yl)ethanamine (38 mg, 0.24 mmol), Cs₂CO₃ (156 mg, 0.48 mmol), Xant-phos (14 mg, 0.024 mmol), and Pd₂(dba)₃ (24 mg, 0.028 mmol) in toluene (5 mL) was stirred at 100° C. for 16 h, after the reaction was completed the mixture was concentrated and purified by prep-HPLC (C18 column, CH₃CN/H₂O, containing 0.05% NH₄HCO₃) to get Compound (I-6) (white solid, 76 mg, 51%). LCMS: 609 (M+H)⁺; HPLC: 100% (254 nm); ¹H NMR (400 MHz, DMSO) δ 10.51 (s, 1H), 8.38 (s, 1H), 8.23 (s, 1H), 8.07 (d, J=8.3 Hz, 1H), 7.96-7.78 (m, 2H), 7.72-7.27 (m, 4H), 6.82 (s, 1H), 6.20 (t, J=5.2 Hz, 1H), 4.04 (s, 3H), 3.36 (s, 2H), 2.62 (t, J=6.4 Hz, 2H), 2.51-2.25 (m, 10H), 2.18 (s, 3H), 0.99 (t, J=7.2 Hz, 3H).

### Compound (I-7)

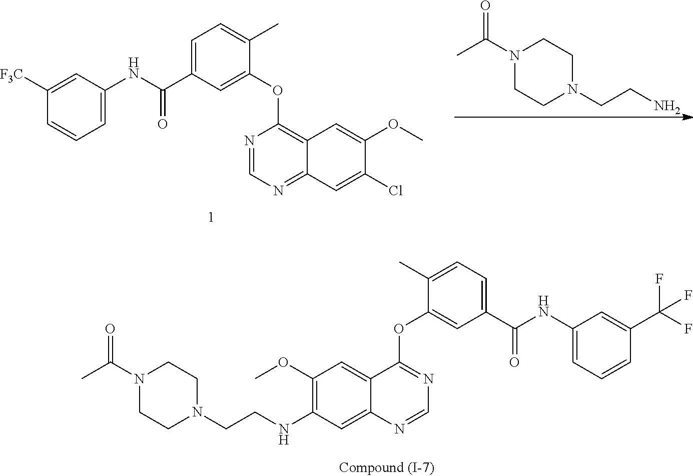

### 3-(7-(2-(4-acetylpiperazin-1-yl)ethylamino)-6-methoxyquinazolin-4-yloxy)-4-methyl-N-(3-(trifluoromethyl)phenyl)benzamide (Compound (I-7))

The mixture of 3-(7-chloro-6-methoxyquinazolin-4-yloxy)-4-methyl-N-(3-(trifluoromethyl)phenyl)benzamide (100 mg, 0.205 mmol), 1-(4-(2-aminoethyl)piperazin-1-yl)ethanone (53 mg, 0.308 mmol), Cs₂CO₃ (133 mg, 0.0205 mmol), Xant-phos (12 mg, 0.0205 mmol), and Pd₂(dba)₃ (18 mg, 0.41 mmol) in toluene (5 mL) was stirred at 100° C. for 16 h, after the reaction was completed the mixture was concentrated and purified by prep-HPLC (C18 column, CH₃CN/H₂O, containing 0.05% NH₄HCO₃) to get Compound (I-7) (white solid, 20 mg, 15.6%). LCMS: 623 (M+H)+; HPLC: 100% (@254 nm); ¹H NMR (400 MHz, DMSO) δ 10.51 (s, 1H), 8.38 (s, H), 8.23 (s, 1H), 8.07 (d, J=8.5 Hz, 1H), 7.94-7.77 (m, 2H), 7.66-7.38 (m, 4H), 6.84 (s, 1H), 6.25 (t, J=5.3 Hz, 1H), 4.04 (s, 3H), 3.41 (ddd, J=20.7, 12.3, 6.7 Hz, 6H), 2.65 (t, J=6.4 Hz, 2H), 2.48-2.34 (m, 4H), 2.18 (s, 3H), 2.00 (s, 3H).

### Compound (I-8)

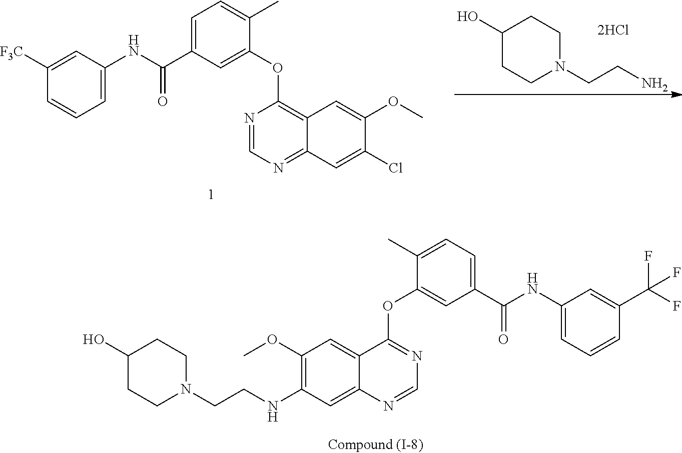 [SMILES: COc1cc2c(Oc3cc(C(=O)Nc4cccc(C(F)(F)F)c4)ccc3C)ncnc2cc1Cl ; NCCN1CCC(O)CC1 ; COc1cc2c(Oc3cc(C(=O)Nc4cccc(C(F)(F)F)c4)ccc3C)ncnc2cc1NCCN1CCC(O)CC1]

### 3-(7-(2-(4-hydroxypiperidin-1-yl)ethylamino)-6-methoxyquinazolin-4-yloxy)-4-methyl-N-(3-(trifluoromethyl)phenyl)benzamide (Compound (I-8))

The mixture of 3-(7-chloro-6-methoxyquinazolin-4-yloxy)-4-methyl-N-(3-(trifluoromethyl)phenyl)benzamide (120 mg, 0.246 mmol), 1-(2-aminoethyl)piperidin-4-ol hydrochloride (53 mg, 0.246 mmol), Cs₂CO₃ (159 mg, 0.246 mmol), Xant-phos (12 mg, 0.025 mmol), and Pd₂(dba)₃ (18 mg, 0.025 mmol) in toluene (5 mL) was stirred at 100° C. for 16 h, after the reaction was completed the mixture was concentrated and purified by prep-HPLC (C18 column, CH₃CN/H₂O, containing 0.05% NH₄HCO₃) to get Compound (I-8) (white solid, 20 mg, 13.6%). LCMS: 596 (M+H)+; HPLC: 100% (@254 nm); ¹H NMR (400 MHz, DMSO) δ 10.50 (s, 1H), 8.38 (s, 1H), 8.23 (s, 1H), 8.07 (d, J=8.3 Hz, 1H), 7.95-7.77 (m, 2H), 7.66-7.29 (m, 4H), 6.81 (s, 1H), 6.20 (t, J=5.1 Hz, 1H), 4.57 (d, J=4.1 Hz, 1H), 4.03 (s, 3H), 3.47 (d, J=4.3 Hz, 3H), 2.77 (d, J=11.3 Hz, 2H), 2.59 (t, J=6.4 Hz, 2H), 2.18 (s, 3H), 2.15-1.92 (m, 2H), 1.74 (d, J=9.9 Hz, 2H), 1.50-1.09 (m, 2H).

### Compound (I-9)

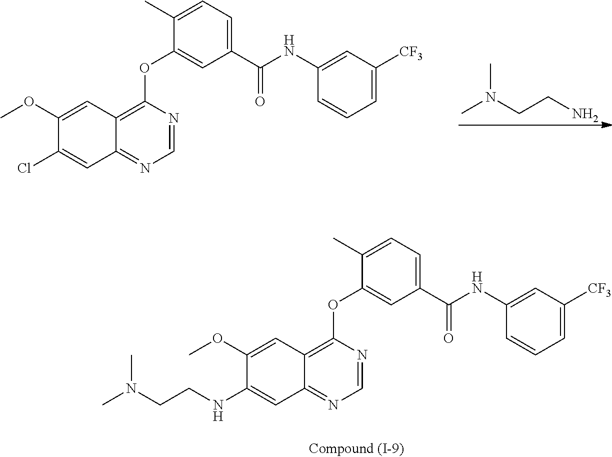

### 3-(7-(2-(dimethylamino)ethylamino)-6-methoxyquinazolin-4-yloxy)-4-methyl-N-(3-(trifluoromethyl)phenyl)benzamide (Compound (I-9))

The mixture of 3-(7-chloro-6-methoxyquinazolin-4-yloxy)-4-methyl-N-(3-(trifluoromethyl)phenyl)benzamide (80 mg, 0.16 mmol), K₂CO₃ (44 mg, 0.32 mmol), Pd₂(dba)₃ (15 mg, 0.016 mmol), x-Phos (8 mg, 0.016 mmol) and N1,N1-dimethylethane-1,2-diamine (14 mg, 0.16 mmol) in toluene (5 mL) was stirred at 90° C. for 16 h, after the reaction was completed, the reaction mixture was concentrated and purified by prep-HPLC (C18 column, CH₃CN/H₂O, containing 0.05% NH₄HCO₃) to get Compound (I-9) (white solid, 15 mg, 17%). LCMS: 540 (M+H)⁺; HPLC: 100% (@254 nm); ¹H NMR (400 MHz, DMSO) δ 10.51 (s, 1H), 8.38 (s, 1H), 8.23 (s, 1H), 8.07 (d, J=8.8 Hz, 1H), 7.94-7.75 (m, 2H), 7.64-7.51 (m, 2H), 7.45 (d, J=8.9 Hz, 2H), 6.82 (s, 1H), 6.10 (s, 1H), 4.03 (s, 3H), 3.33 (s, 2H), 2.56 (t, J=6.2 Hz, 2H), 2.22 (s, 6H), 2.18 (s, 3H).

### Compound (I-10)

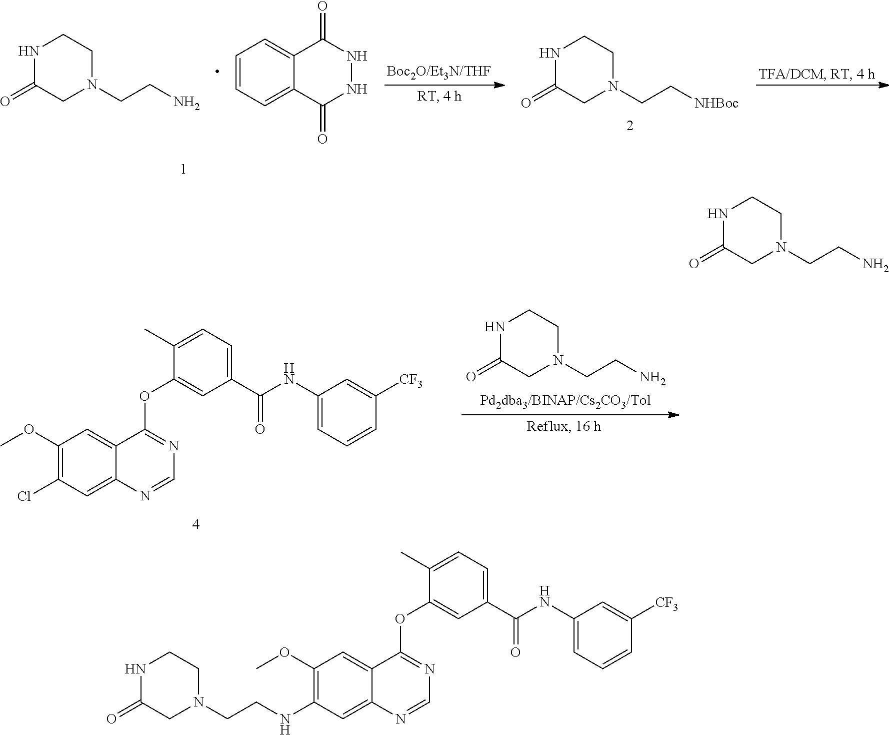

### tert-butyl 2-(3-oxopiperazin-1-yl)ethylcarbamate (2)

Complex compound of 4-(2-aminoethyl)piperazin-2-one with 2,3-dihydrophthalazine-1,4-dione was synthesized according to reference (Molecules, 2003, 8(7), p 565-592). The mixture of this complex compound (4.5 g, 14.74 mmol), (Boc)₂O (4.82 g, 22.11 mmol) and Et₃N (7.46 g, 73.71 mmol) in THF (110 mL) was stirred at RT for 4 h. The mixture was concentrated in vacuum, the residue was diluted with brine (500 mL) and extracted with DCM (500 mL×3), the combined organic phase was washed with brine (200 mL), dried over anhydrous Na₂SO₄, filtered, concentrated and purified by preparative HPLC (C18 column, CH₃CN/H₂O, containing 0.05% NH₄HCO₃) to give tert-butyl 2-(3-oxopiperazin-1-yl)ethylcarbamate as white solid (2 g, yield 56%). LCMS (m/z):244 [M+H]⁺.

### 4-(2-aminoethyl)piperazin-2-one (3)

The mixture of tert-butyl 2-(3-oxopiperazin-1-yl)ethylcarbamate (300 mg, 1.23 mmol) and TFA (1.4 g, 12.35 mmol) in THF (100 mL) was stirred at RT for 4 h. The mixture was concentrated in vacuum to leave crude 4-(2-aminoethyl)piperazin-2-one as white solid (150 mg, yield 56%). LCMS (m/z):144 [M+H]⁺.

### 3-(6-methoxy-7-(2-(3-oxopiperazin-1-yl)ethylamino)quinazolin-4-yloxy)-4-methyl-N-(3-(trifluoromethyl)phenyl)benzamide (Compound (I-10))

The mixture of 4-(2-aminoethyl)piperazin-2-one (150 mg, 1.05 mmol), 3-(7-chloro-6-methoxyquinazolin-4-yloxy)-4-methyl-N-(3-(trifluoromethyl)phenyl)benzamide (78 mg, 0.16 mmol), Pd₂(dba)₃ (9.3 mg, 0.01 mmol), BINAP (1.4 g, 12.45 mmol) and t-BuOK (89.3 mg, 0.80 mmol) in toluene (10 mL) was stirred at 100° C. for 16 h. The mixture was diluted with water (200 mL) and extracted with DCM (100 mL×2), the combined organic was dried over anhydrous Na₂SO₄, filtered, concentrated and purified by preparative HPLC (C18 column, CH₃CN/H₂O, containing 0.05% NH₄HCO₃) to give Compound (I-10) as white solid (20.7 mg, 22%).

LCMS (m/z): 595.2 [M+H]⁺; ¹H NMR (400 MHz, DMSO-d₆) δ (PPM): 8.50 (s, 1H), 8.40 (s, 1H), 7.88 (s, 1H), 7.79 (d, J=8.0 Hz, 1H), 7.68 (t, J=6 Hz, 2H), 7.36-7.30 (m, 4H), 6.79 (s, 1H), 6.09 (s, 1H), 5.50 (t, J=4 Hz, 1H), 3.97 (s, 3H), 3.40-3.20 (m, 4H), 3.16 (s, 2H), 2.75 (t, J=6 Hz, 2H), 2.67 (t, J=4 Hz, 2H), 2.17 (s, 3H).

### Compound (I-11)

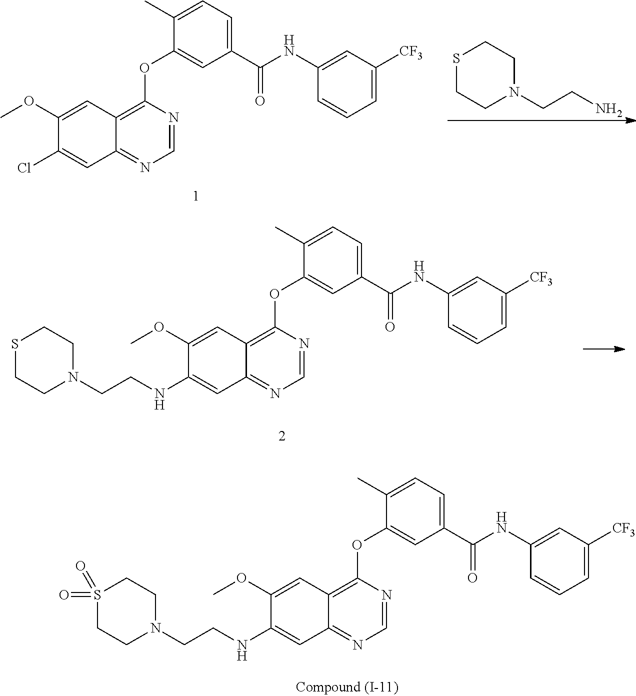 [SMILES: COc1cc2c(Oc3cc(C(=O)Nc4cccc(C(F)(F)F)c4)ccc3C)ncnc2cc1Cl ; NCCN1CCSCC1 ; COc1cc2c(Oc3cc(C(=O)Nc4cccc(C(F)(F)F)c4)ccc3C)ncnc2cc1NCCN1CCSCC1 ; COc1cc2c(Oc3cc(C(=O)Nc4cccc(C(F)(F)F)c4)ccc3C)ncnc2cc1NCCN1CCS(=O)(=O)CC1]

### 3-(6-methoxy-7-(2-thiomorpholinoethylamino)quinazolin-4-yloxy)-4-methyl-N-(3-(trifluoromethyl)phenyl)benzamide (2)

The mixture of 3-(7-chloro-6-methoxyquinazolin-4-yloxy)-4-methyl-N-(3-(trifluoromethyl)phenyl)benzamide (100 mg, 0.206 mmol), K₂CO₃ (56 mg, 0.412 mmol), Pd₂(dba)₃ (18 mg, 0.02 mmol), x-Phos (10 mg, 0.02 mmol) and 2-thiomorpholinoethanamine (30 mg, 0.206 mmol) in toluene (5 mL) was stirred at 90° C. for 16 h, after the reaction was completed the mixture was concentrated and purified by silica gel (DCM/MeOH=10/1) to obtain title compound 2 (yellow solid, 120 mg, 97% yield). LCMS: 598 (M+H)⁺.

### 3-(6-methoxy-7-(2-thiomorpholinoethylamino)quinazolin-4-yloxy)-4-methyl-N-(3-(trifluoromethyl)phenyl)benzamide 1,1-dioxide (Compound (I-11))

The mixture of 3-(6-methoxy-7-(2-thiomorpholinoethylamino)quinazolin-4-yloxy)-4-methyl-N-(3-(trifluoromethyl)phenyl)benzamide (60 mg, 0.1 mmol), oxone (184 mg, 0.3 mmol) in MeOH (5 mL) and H₂O (2 mL) was stirred at rt for 16 h, after completion the mixture was washed with sat.NaHSO₃ solution (100 mL), extracted with EA (100 mL), concentrated and purified by prep-HPLC (C18 column, CH₃CN/H₂O, containing 0.05% NH₄HCO₃) to get Compound (I-11) (white solid, 20 mg, 27%). LCMS: 630 (M+H)⁺; HPLC: 100% (@254 nm); ¹H NMR (400 MHz, DMSO) δ 10.51 (s, 1H), 8.38 (s, 1H), 8.23 (s, 1H), 8.07 (d, J=8.2 Hz, 1H), 7.97-7.78 (m, 2H), 7.67-7.51 (m, 2H), 7.45 (d, J=7.9 Hz, 2H), 6.85 (s, 1H), 6.25 (t, J=5.4 Hz, 1H), 4.04 (s, 3H), 3.50-3.32 (m, 2H), 3.09 (t, J=14.1 Hz, 4H), 3.03 (s, 4H), 2.83 (t, J=6.3 Hz, 2H), 2.18 (s, 3H).

### Compound (I-15)

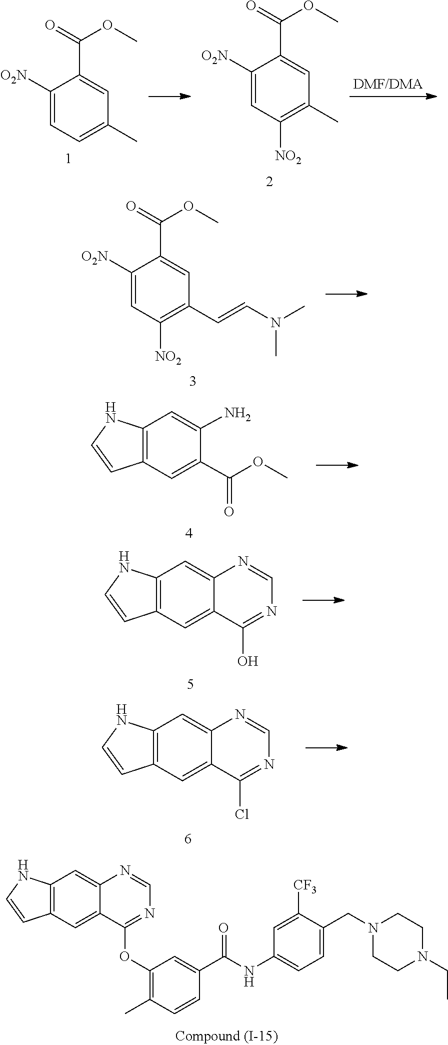

### Methyl 6-nitro-1H-benzo[d]imidazole-5-carboxylate (2)

The mixture of methyl 5-methyl-2-nitrobenzoate (4.5 g, 23 mmol), HNO₃ (2 mL) and H₂SO₄ (4 mL) was stirred at 0° C. for 3 h, after the reaction was completed the mixture was concentrated and purified by silica gel (DCM/MeOH=10/1) to obtain title compound 2 (yellow solid, 2.5 g, 45% yield). LCMS: 263 (M+Na)⁺.

### (E)-methyl 5-(2-(dimethylamino)vinyl)-2,4-dinitrobenzoate (3)

The mixture of methyl 5-methyl-2,4-dinitrobenzoate (2 g, 8.83 mmol), DMF-DMA (990 mg, 8.83 mmol) and dioxane (40 mL) was stirred at 115° C. for 3 h, after completion, was used for next step. LCMS: 296 (M+H)⁺.

### Methyl 6-amino-1H-indole-5-carboxylate (4)

The mixture of (E)-methyl 5-(2-(dimethylamino)vinyl)-2,4-dinitrobenzoate (2.2 g, 7.45 mmol), Pd/C (220 mg) in MeOH (40 mL) was stirred at rt for 3 h, after completion, was concentrated to obtain title compound 4 (yellow solid, 1.3 g, 92% yield). LCMS: 191 (M+H)⁺.

### 8H-pyrrolo[3,2-g]quinazolin-4-ol (5)

The mixture of methyl 6-amino-1H-indole-5-carboxylate (1.3 g, 6.84 mmol) in HCONH₂ (25 mL) was stirred at 140° C. for 13 h, after the reaction was completed the mixture was concentrated and purified by silica gel (DCM/MeOH=10/1) to obtain title compound 5 (yellow solid, 800 mg, 63% yield). LCMS: 186 (M+H)⁺.

### 4-chloro-8H-pyrrolo[3,2-g]quinazoline (6)

The mixture of 8H-pyrrolo[3,2-g]quinazolin-4-ol (200 mg, 1.05 mmol), POCl₃ (0.1 mL) and DIPEA (0.4) in DCE (10 mL) was stirred at 80° C. for 3 h, after the reaction was completed the mixture was concentrated and purified by silica gel (PE/EA=10/1) to obtain title compound 6 (yellow solid, 70 mg, 32% yield). LCMS: 204 (M+H)⁺.

### 3-(8H-pyrrolo[3,2-g]quinazolin-4-yloxy)-N-(4-((4-ethylpiperazin-1-yl)methyl)-3-(trifluoromethyl)phenyl)-4-methylbenzamide (Compound (I-15))

The mixture of N-(4-((4-ethylpiperazin-1-yl)methyl)-3-(trifluoromethyl)phenyl)-3-hydroxy-4-methylbenzamide (164 mg, 0.39 mmol), 4-chloro-8H-pyrrolo[3,2-g]quinazoline (80 mg, 0.39 mmol) and Cs₂CO₃ (383 mg, 1.18 mmol) in DCE (10 mL) was stirred at 80° C. for 3 h, after the reaction was completed the mixture was concentrated and purified by silica gel (DCM/MeOH=10/1) to obtain Compound (I-15) (yellow solid, 11 mg, 5% yield). LCMS: 589 (M+H)⁺. ¹H NMR (400 MHz, DMSO) δ 11.70 (s, 1H), 10.47 (s, 1H), 8.72 (s, 1H), 8.54 (s, 1H), 8.18 (d, J=2.0 Hz, 1H), 8.05 (d, J=8.6 Hz, 1H), 7.98 (s, 1H), 7.92 (dd, J=10.2, 2.4 Hz, 2H), 7.83 (d, J=3.3 Hz, 1H), 7.70 (d, J=8.6 Hz, 1H), 7.58 (d, J=8.0 Hz, 1H), 6.84 (d, J=3.1 Hz, 1H), 3.55 (s, 2H), 2.45-2.24 (m, 10H), 2.14 (d, J=53.9 Hz, 3H), 0.97 (t, J=7.2 Hz, 3H).

### Compound (I-16)

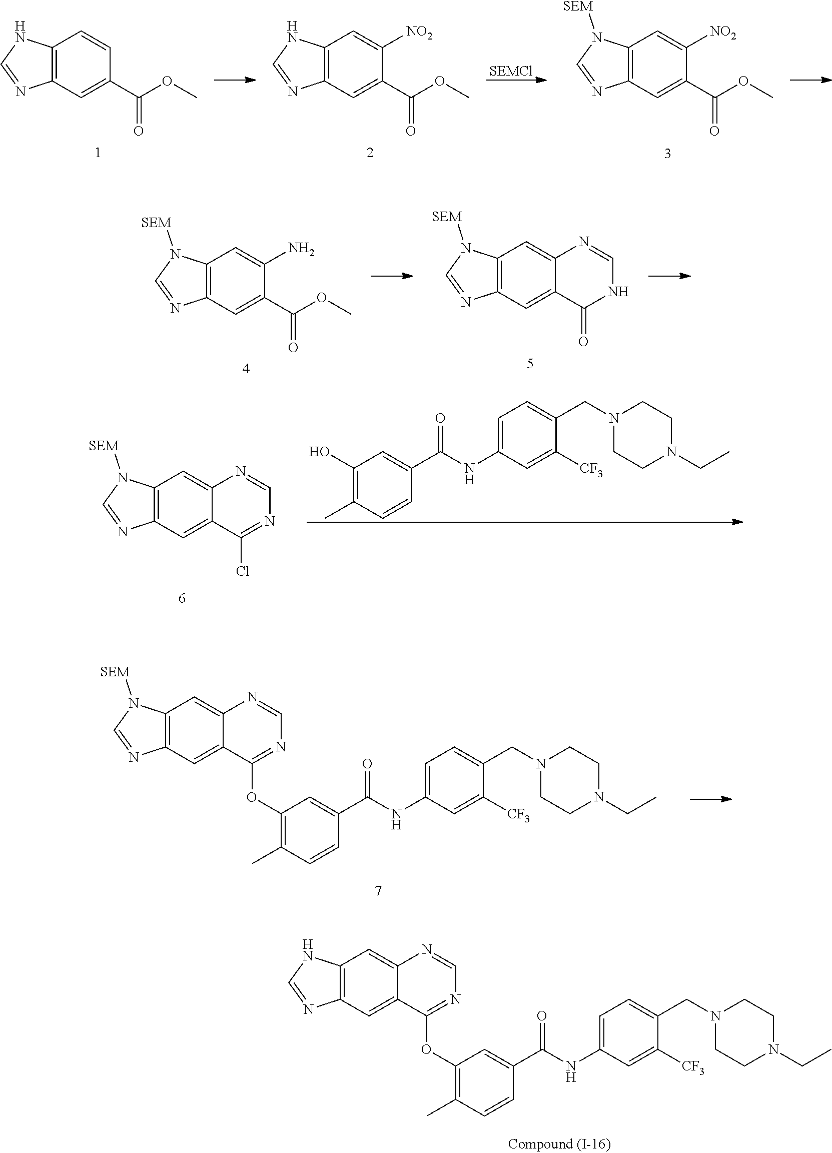

### Methyl 6-nitro-1H-benzo[d]imidazole-5-carboxylate (2)

The mixture of methyl 1H-benzo[d]imidazole-5-carboxylate (2 g, 11.36 mmol), HNO₃ (2 mL) and H₂SO₄ (4 mL) was stirred at 0° C. for 3 h, after the reaction was completed the mixture was concentrated and purified by silica gel (DCM/MeOH=10/1) to obtain title compound 2 (yellow solid, 1.6 g, 64% yield). LCMS: 222 (M+H)⁺.

### Methyl 6-nitro-1-((2-(trimethylsilyl)ethoxy)methyl)-1H-benzo[d]imidazole-5-carboxylate (3)

The mixture of methyl 6-nitro-1H-benzo[d]imidazole-5-carboxylate (1.6 g, 7.2 mmol), SEMCl (1.8 g, 10.8 mmol) and NaH (60%) (720 mg, 18 mmol) in THF (100 mL) was stirred at 0° C. for 3 h, after the reaction was completed the mixture was concentrated and purified by silica gel (PE/EA=10/1) to obtain title compound 32 (yellow oil, 1.5 g, 60% yield). LCMS: 352 (M+H)⁺.

### Methyl 6-amino-1-((2-(trimethylsilyl)ethoxy)methyl)-1H-benzo[d]imidazole-5-carboxylate (4)

The mixture of methyl 6-nitro-1-((2-(trimethylsilyl)ethoxy)methyl)-1H-benzo[d]imidazole-5-carboxylate (1.6 g, 4.56 mmol), Zn (3 g, 45.6 mmol)) in AcOH (25 mL) was stirred at 0° C. for 3 h, after the reaction was completed the mixture was concentrated and purified by silica gel (DCM/MeOH=10/1) to obtain title compound 4 (yellow solid, 1.2 g, 82% yield). LCMS: 322 (M+H)⁺.

### 3-((2-(trimethylsilyl)ethoxy)methyl)-3H-imidazo[4,5-g]quinazolin-8(7H)-one (5)

The mixture of methyl 6-amino-1-((2-(trimethylsilyl)ethoxy)methyl)-1H-benzo[d]imidazole-5-carboxylate (1.2 g, 3.73 mmol) in HCONH₂ (25 mL) was stirred at 140° C. for 13 h, after the reaction was completed the mixture was concentrated and purified by silica gel (DCM/MeOH=10/1) to obtain title compound 5 (yellow solid, 800 mg, 68% yield). LCMS: 317 (M+H)⁺.

### 8-chloro-3-((2-(trimethylsilyl)ethoxy)methyl)-3H-imidazo[4,5-g]quinazoline (6)

The mixture of 3-((2-(trimethylsilyl)ethoxy)methyl)-3H-imidazo[4,5-g]quinazolin-8(7H)-one (400 mg, 1.26 mmol), POCl₃ (0.1 mL) and DIPEA (0.4) in DCE (10 mL) was stirred at 80° C. for 3 h, after the reaction was completed the mixture was concentrated and purified by silica gel (PE/EA=10/1) to obtain title compound 6 (yellow solid, 180 mg, 42% yield). LCMS: 335 (M+H)⁺.

### N-(4-((4-ethylpiperazin-1-yl)methyl)-3-(trifluoromethyl)phenyl)-4-methyl-3-(3-((2-(trimethylsilyl)ethoxy)methyl)-3H-imidazo[4,5-g]quinazolin-8-yloxy)benzamide (7)

The mixture of N-(4-((4-ethylpiperazin-1-yl)methyl)-3-(trifluoromethyl)phenyl)-3-hydroxy-4-methylbenzamide (126 mg, 0.299 mmol), 8-chloro-3-((2-(trimethylsilyl)ethoxy)methyl)-3H-imidazo[4,5-g]quinazoline (100 mg, 0.299 mmol) and Cs₂CO₃ (195 mg, 0.599 mmol) in DCE (10 mL) was stirred at 80° C. for 3 h, after the reaction was completed the mixture was concentrated and purified by silica gel (DCM/MeOH=10/1) to obtain title compound 7 (yellow solid, 40 mg, 19% yield). LCMS: 720 (M+H)⁺.

### 3-(3H-imidazo[4,5-g]quinazolin-8-yloxy)-N-(4-((4-ethylpiperazin-1-yl)methyl)-3-(trifluoromethyl)phenyl)-4-methylbenzamide (Compound (I-16))

The mixture of N-(4-((4-ethylpiperazin-1-yl)methyl)-3-(trifluoromethyl)phenyl)-4-methyl-3-(3-((2-(trimethylsilyl)ethoxy)methyl)-3H-imidazo[4,5-g]quinazolin-8-yloxy)benzamide (35 mg, 0.049 mmol), TBAF (1.0 M) (0.3 mL) in THF (4 mL) was stirred at 70° C. for 16 h, after the reaction was completed the mixture was concentrated and purified by silica gel (DCM/MeOH=10/1) to obtain Compound (I-16) (white solid, 4 mg, 14% yield). LCMS: 590 (M+H)⁺. ¹H NMR (400 MHz, DMSO) δ 10.48 (s, 1H), 8.73 (s, 1H), 8.67 (s, 1H), 8.62 (s, 1H), 8.28-8.13 (m, 2H), 8.05 (d, J=8.5 Hz, 1H), 7.93 (dd, J=11.6, 3.7 Hz, 2H), 7.70 (d, J=8.6 Hz, 1H), 7.59 (d, J=8.1 Hz, 1H), 3.52 (d, J=23.0 Hz, 2H), 2.43-2.25 (m, 10H), 2.22 (s, 3H), 0.97 (t, J=7.2 Hz, 3H).

### Compound (I-19)

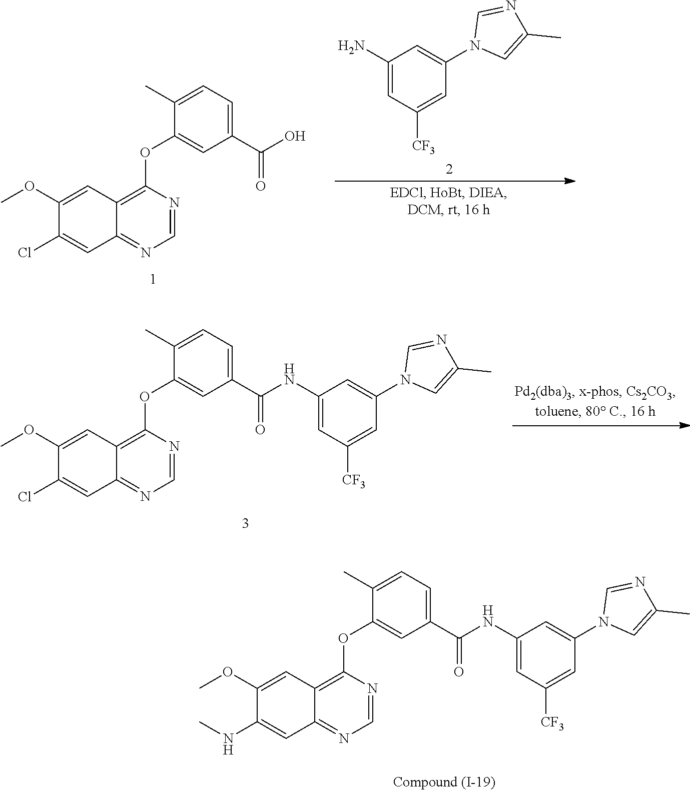

### 3-(7-chloro-6-methoxyquinazolin-4-yloxy)-4-methyl-N-(3-(4-methyl-1H-imidazol-1-yl)-5-(trifluoromethyl)phenyl)benzamide (3)

The mixture of 3-(7-chloro-6-methoxyquinazolin-4-yloxy)-4-methylbenzoic acid (756 mg, 2.194 mmol), 3-(4-methyl-1H-imidazol-1-yl)-5-(trifluoromethyl)aniline (481 mg, 1.994 mmol), EDCI (764 mg, 3.988 mmol), HOBt (404 mg, 2.991 mmol) and DIEA (2 mL) in DCM (20 mL) was stirred at rt for 16 h, after completion, the mixture was concentrated, the residue was diluted with water (100 mL) and extracted with EtOAc (100 mL), the organic was washed with saturated brine (50 mL×2), dried over anhydrous Na₂SO₄, concentrated and purified by column chromatography (silica gel, DCM:MeOH=20:1) to obtain target compound (486 mg, isolated yield 43%) as solid. LCMS (m/z): 567.9 [M+H]⁺.

### 3-(6-methoxy-7-(methylamino)quinazolin-4-yloxy)-4-methyl-N-(3-(4-methyl-1H-imidazol-1-yl)-5-(trifluoromethyl)phenyl)benzamide (Compound (I-19))

The mixture of 3-(7-chloro-6-methoxyquinazolin-4-yloxy)-4-methyl-N-(3-(4-methyl-1H-imidazol-1-yl)-5-(trifluoromethyl)phenyl)benzamide (50 mg, 0.088 mmol), CH₃NH₂—HCl (59 mg, 0.88 mmol), Pd₂(dba)₃ (12 mg, 0.013 mmol), X-Phos (13 mg, 0.026 mmol) and Cs₂CO₃ (344 mg, 1.06 mmol) in toluene (10 mL) was purged with N₂ for 5 times, and then heated at 80° C. for 16 h, the mixture was concentrated and purified by preparative HPLC to get the desired product (1 mg) as solid. LCMS (m/z): 563.0 [M+H]⁺. ¹H NMR (400 MHz, DMSO-d₆) δ (PPM): 8.50 (d, J=3.6 Hz, 1H), 8.19 (d, J=3.6 Hz, 1H), 7.82 (s, 1H), 7.78 (d, J=8.4 Hz, 1H), 7.71 (s, 2H), 7.47 (d, J=8.0 Hz, 1H), 7.39-7.30 (m, 3H), 7.07 (s, 1H), 6.88 (s, 1H), 5.02 (d, J=5.6 Hz, 1H), 4.04 (s, 3H), 3.04 (d, J=5.2 Hz, 3H), 2.29-2.17 (m, 6H).

### Compound (I-20)

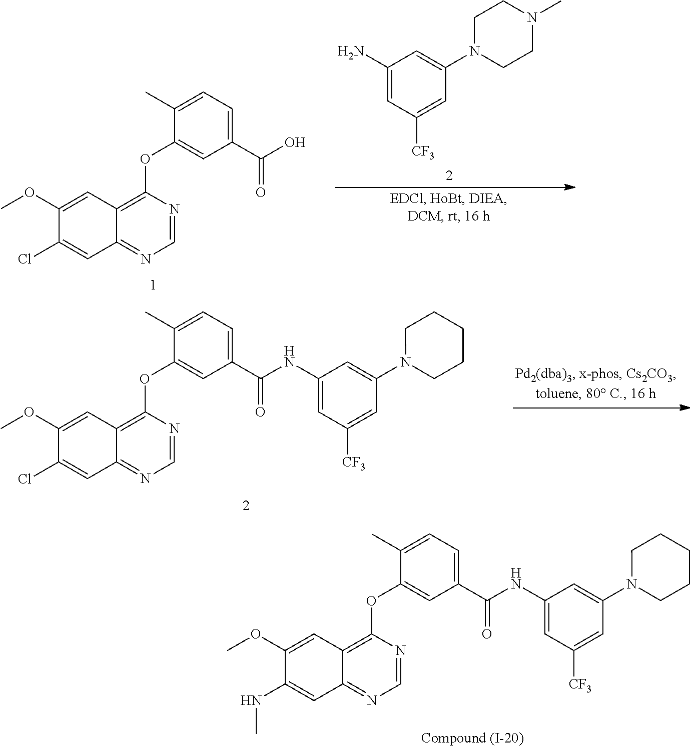 [SMILES: COc1cc2c(Oc3cc(C(=O)O)ccc3C)ncnc2cc1Cl ; CN1CCN(c2cc(N)cc(C(F)(F)F)c2)CC1 ; COc1cc2c(Oc3cc(C(=O)Nc4cc(N5CCCCC5)cc(C(F)(F)F)c4)ccc3C)ncnc2cc1Cl ; CNc1cc2ncnc(Oc3cc(C(=O)Nc4cc(N5CCCCC5)cc(C(F)(F)F)c4)ccc3C)c2cc1OC]

### 3-(7-chloro-6-methoxyquinazolin-4-yloxy)-4-methyl-N-(3-(4-methylpiperazin-1-yl)-5-(trifluoromethyl)phenyl)benzamide (3)

The a stirred mixture of 3-(7-chloro-6-methoxyquinazolin-4-yloxy)-4-methylbenzoic acid (285 mg, 0.827 mmol) and HOBt (153 mg, 1.128 mmol) in DCM/THF (1:1, 6 mL) was added DIEA (1 mL) and EDCI (288 mg, 1.504 mmol), the resulting mixture was stirred at rt for 10 min, and then 3-(4-methylpiperazin-1-yl)-5-(trifluoromethyl)aniline (195 mg, 0.752 mmol) was added. The mixture was stirred at rt for 16 h and concentrated in vacuum, the residue was diluted with EtOAc (50 mL), washed with saturated brine (50 mL×2), dried over anhydrous Na₂SO₄, concentrated and purified by column chromatography (silica gel, DCM:MeOH=20:1) to get desired product as solid (147 mg, isolated yield 30%). LCMS (m/z): 586.1 [M+H]⁺.

### 3-(6-methoxy-7-(methylamino)quinazolin-4-yloxy)-4-methyl-N-(3-(4-methylpiperazin-1-yl)-5-(trifluoromethyl)phenyl)benzamide (Compound (I-20))

The mixture of 3-(7-chloro-6-methoxyquinazolin-4-yloxy)-4-methyl-N-(3-(4-methylpiperazin-1-yl)-5-(trifluoromethyl)phenyl)benzamide (105 mg, 0.179 mmol), CH₃NH₂—HCl (121 mg, 1.790 mmol), Pd₂(dba)₃ (15 mg, 0.018 mmol), X-Phos (26 mg, 0.054 mmol) and Cs₂CO₃ (700 mg, 2.148 mmol) in toluene (10 mL) was purged with N₂ for 5 times, and then stirred at 80° C. for 16 h, the mixture was concentrated and purified by preparative HPLC to get the desired product as solid (19.06 mg, isolated yield 18%). LCMS (m/z): 581.0 [M+H]⁺. ¹H NMR (400 MHz, DMSO-d₆) δ (PPM) 10.28 (s, 1H), 8.37 (s, 1H), 7.85-7.90 (m, 2H), 7.68 (s, 1H), 7.62 (s, 1H), 7.55 (d, J=7.6 Hz, 1H), 7.44 (s, 1H), 6.95 (s, 1H), 6.72 (s, 1H), 6.58-6.54 (m, 1H), 4.02 (s, 3H), 3.21 (br, 4H), 2.89 (d, J=4.8 Hz, 3H), 2.51-2.45 (m, 4H), 2.22 (s, 3H), 2.18 (s, 3H).

### Compound (I-21)

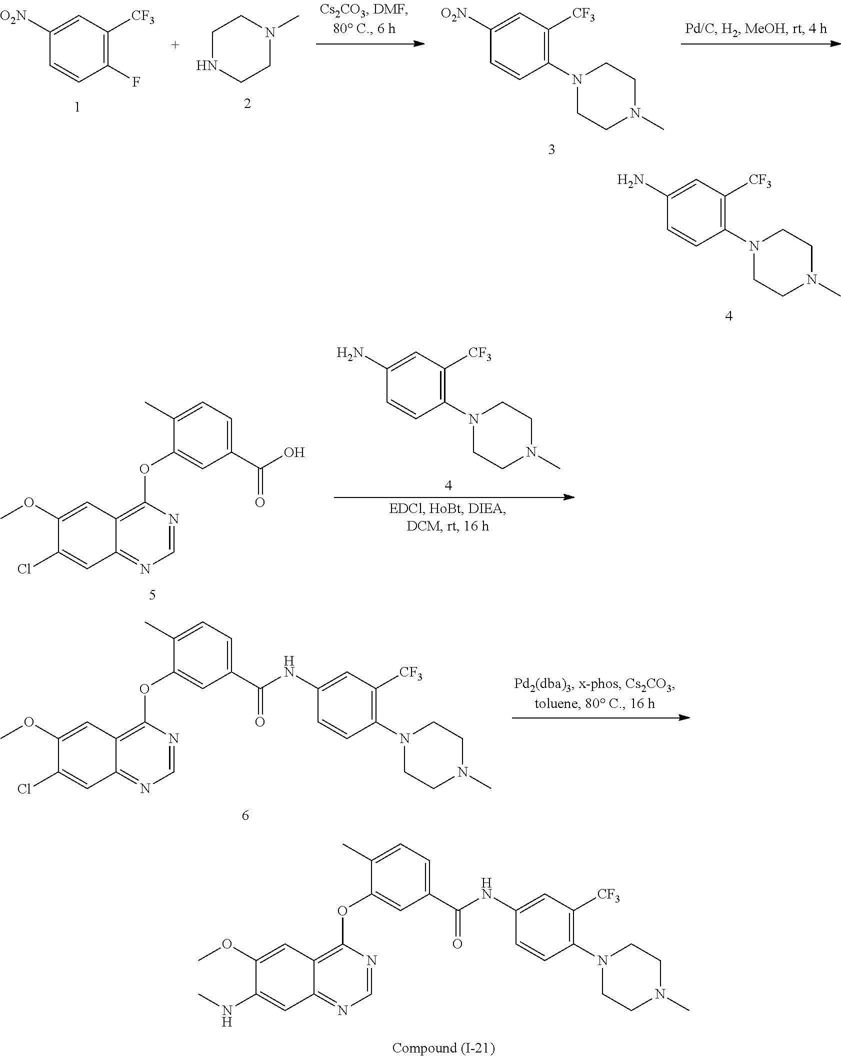

### 1-methyl-4-(4-nitro-2-(trifluoromethyl)phenyl)piperazine (3)

The mixture of 1-fluoro-4-nitro-2-(trifluoromethyl)benzene (2000 mg, 9.565 mmol), 1-methylpiperazine (958 mg, 9.565 mmol) and Cs₂CO₃ (6232 mg, 19.130 mmol) in DMF (30 mL) was stirred at 80° C. for 6 h, the mixture was cooled down to rt, diluted with water (100 mL) and extracted with EtOAc (50 mL×2), the combined organic was washed with saturated brines (50 mL×5), dried over anhydrous Na₂SO₄, concentrated and purified by column chromatography (silica gel, DCM:MeOH=20:1) to obtain target compound (1190 mg, isolated yield 43%) as solid. LCMS (m/z): 290.0 [M+H]⁺.

### 4-(4-methylpiperazin-1-yl)-3-(trifluoromethyl)aniline (4)

The mixture of 1-methyl-4-(4-nitro-2-(trifluoromethyl)phenyl)piperazine (1050 mg, 3.630 mmol) and Pd/C (10%, 77 mg, 0.726 mmol) in MeOH (40 mL) was purged with H₂ for 5 times, and then stirred at rt under H₂ (1 atm) for 4 h, the mixture was filtered through Celite™, the filtrate was concentrated to leave crude product (550 mg, yield 58%) as oil. LCMS (m/z): 260.1 [M+H]⁺.

### 3-(7-chloro-6-methoxyquinazolin-4-yloxy)-4-methyl-N-(4-(4-methylpiperazin-1-yl)-3-(trifluoromethyl)phenyl)benzamide (6)

The mixture of 3-(7-chloro-6-methoxyquinazolin-4-yloxy)-4-methylbenzoic acid (512 mg, 1.485 mmol), 4-(4-methylpiperazin-1-yl)-3-(trifluoromethyl)aniline (350 mg, 1.350 mmol), EDCI (518 mg, 2.70 mmol) and HOBt (274 mg, 2.025 mmol) in DCM/THF (1:1, 10 mL) was stirred at rt for 16 h. The mixture was concentrated in vacuum, the residue was diluted with EtOAc (100 mL), washed with saturated brines (50 mL×2), dried over anhydrous Na₂SO₄, filtered and concentrated to leave crude product as solid (403 mg, isolated yield 51%). LCMS (m/z): 585.9 [M+H]⁺.

### 3-(6-methoxy-7-(methylamino)quinazolin-4-yloxy)-4-methyl-N-(4-(4-methylpiperazin-1-yl)-3-(trifluoromethyl)phenyl)benzamide (Compound (I-21))

The mixture of 3-(7-chloro-6-methoxyquinazolin-4-yloxy)-4-methyl-N-(4-(4-methylpiperazin-1-yl)-3-(trifluoromethyl)phenyl)benzamide (50 mg, 0.085 mmol), CH₃NH₂—HCl (58 mg, 0.85 mmol), Pd₂(dba)₃ (12 mg, 0.013 mmol), X-Phos (12 mg, 0.026 mmol) and Cs₂CO₃ (332 mg, 1.02 mmol) in toluene (20 mL) was purged with N₂ for 5 times and stirred at 80° C. for 16 h. The mixture was diluted with EtOAc (50 mL), washed with saturated brine (50 mL×2), dried over anhydrous Na₂SO₄, concentrated and purified by preparative HPLC to afford desired product as white solid (5.24 mg, isolated yield 11%). LCMS (m/z): 581.0 [M+H]⁺. ¹H NMR (400 MHz, DMSO-d₆) δ (PPM) 10.394 (s, 1H), 8.37 (s, 1H), 8.13 (d, J=2.0 Hz, 1H), 8.05 (d, J=8.4 Hz, 1H), 7.88-7.85 (m, 2H), 7.54 (t, J=8.8 Hz, 2H), 7.44 (s, 1H), 6.72 (s, 1H), 6.57 (q, J=4.8 Hz, 1H), 4.02 (s, 3H), 2.89 (d, J=4.8 Hz, 3H), 2.84 (d, J=4.4 Hz, 4H) 2.46 (br, 4H), 2.23 (s, 3H), 2.18 (s, 3H).

### Compound (I-22)

Compound (I-22) was synthesized with similar procedure as Compound (I-1). 1H NMR (400 MHz, DMSO-d6) δ (PPM) 10.666 (s, 1H), 8.373 (s, 1H), 8.13 (d, J=2.0 Hz, 1H), 7.94 (d, J=8.4 Hz, 1H), 7.70 (d, J=8.4 Hz, 2H), 7.54 (d, J=6.4 Hz, 1H), 7.42 (d, J=10.0 Hz, 1H), 7.405 (s, 1H), 6.71 (s, 1H), 6.58-6.54 (m, 1H), 4.012 (s, 3H), 3.552 (s, 2H), 2.87 (d, J=5.2 Hz, 3H), 2.45-2.24 (m, 9H), 2.16 (s, 3H), 0.973 (t, J=7.2 Hz, 3H).

### Compound (I-23)

¹H NMR (400 MHz, DMSO-d₆) δ (PPM) 10.706 (s, 1H), 8.423 (s, 1H), 8.15 (d, J=1.6 Hz, 1H), 7.97 (d, J=8.8 Hz, 1H), 7.78 (d, J=8.4 Hz, 2H), 7.55 (d, J=6.4 Hz, 1H), 7.43 (d, J=10.0 Hz, 1H), 7.417 (s, 1H), 6.71 (s, 1H), 6.686 (br, 1H), 4.019 (s, 3H), 3.762 (s, 2H), 3.17-3.10 (m, 4H), 2.93-2.81 (m, 7H), 2.168 (s, 3H), 0.973 (t, J=7.2 Hz, 3H).

### Compound (I-24)

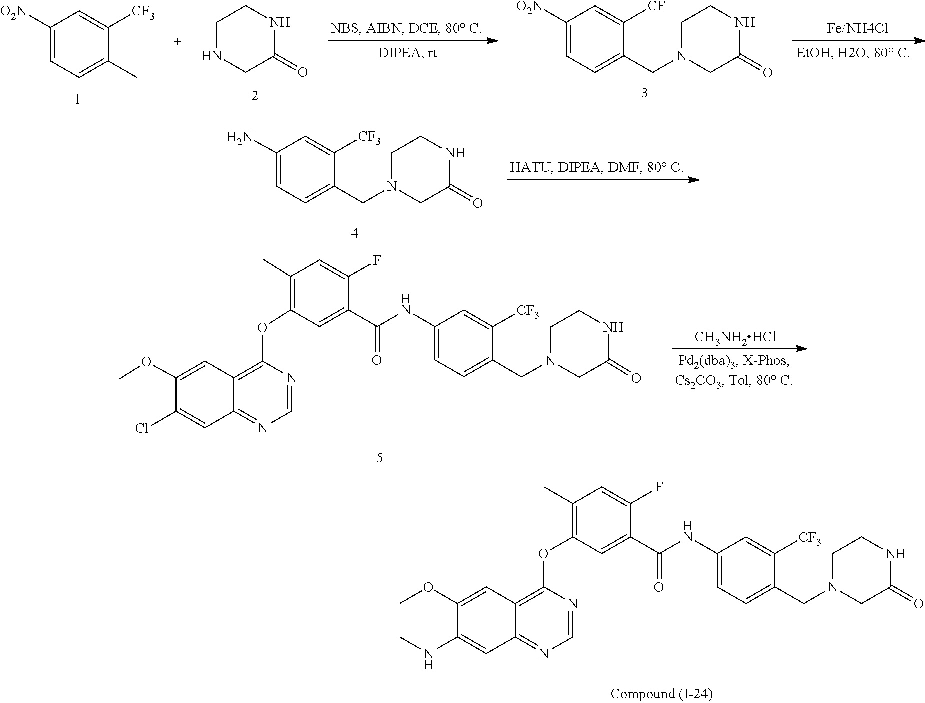

### 4-(4-nitro-2-(trifluoromethyl)benzyl)piperazin-2-one (3)

The mixture of 1-methyl-4-nitro-2-(trifluoromethyl)benzene (1.5 g, 7.3 mmol), NBS (2.0 g, 11.0 mmol) and AIBN (120 mg, 0.73 mmol) in DCE (20 mL) was stirred at 80° C. overnight. The mixture was cooled down to rt, and then a mixture of piperazin-2-one (900 mg, 8.7 mmol) and DIPEA (3.0 mL) was added dropwise, the resulting mixture was stirred at rt for 3 h and concentrated in vacuum, the residue was purified by column chromatography (silica gel, MeOH/DCM= 1/10) to give 4-(4-nitro-2-(trifluoromethyl)benzyl)piperazin-2-one as brown solid (1.8 g, yield 82%). LCMS (m/z): 304 [M+H]⁺.

### 4-(4-amino-2-(trifluoromethyl)benzyl)piperazin-2-one (4)

The mixture of 4-(4-nitro-2-(trifluoromethyl)benzyl)piperazin-2-one (500 mg, 1.65 mmol), Fe (462 mg, 8.25 mmol) and NH₄Cl (462 mg, 8.25 mmol) in EtOH (15 mL) and H₂O (3 mL) was stirred at 80° C. for 2 h. The mixture was cooled down to rt and filtered, the filtrate was concentrated in vacuum, the residue was diluted with ethyl acetate (150 mL), washed with brine (50 mL×2), dried over anhydrous Na₂SO₄, filtered and concentrated to leave crude 4-(4-amino-2-(trifluoromethyl)benzyl)piperazin-2-one as brown oil (400 mg, yield 88%). LCMS (m/z): 274.1 [M+H]⁺.

### 5-(7-chloro-6-methoxyquinazolin-4-yloxy)-2-fluoro-4-methyl-N-(4-((3-oxopiperazin-1-yl)methyl)-3-(trifluoromethyl)phenyl)benzamide (5)

The mixture of 5-(7-chloro-6-methoxyquinazolin-4-yloxy)-2-fluoro-4-methylbenzoic acid (265 mg, 0.73 mmol), 4-(4-amino-2-(trifluoromethyl)benzyl)piperazin-2-one (200 mg, 0.73 mmol), HATU (416 mg, 1.09 mmol) and DIPEA (1.0 mL) in DMF (5.0 mL) was stirred at 80° C. overnight. The mixture was diluted with water (100 mL) and extracted with ethyl acetate (150 mL×2), the combined organic phase was washed with brine (100 mL×2), dried over anhydrous Na₂SO₄, concentrated and purified by column chromatography (silica gel, DCM/MeOH=10/1) to obtain 5-(7-chloro-6-methoxyquinazolin-4-yloxy)-2-fluoro-4-methyl-N-(4-((3-oxopiperazin-1-yl)methyl)-3-(trifluoromethyl)phenyl)benzamide as red solid (80 mg, yield 18%). LCMS (m/z): 617.9 [M+H]⁺.

### 2-fluoro-5-(6-methoxy-7-(methylamino)quinazolin-4-yloxy)-4-methyl-N-(4-((3-oxopiperazin-1-yl)methyl)-3-(trifluoromethyl)phenyl)benzamide (Compound (I-24))

The mixture of 5-(7-chloro-6-methoxyquinazolin-4-yloxy)-2-fluoro-4-methyl-N-(4-((3-oxopiperazin-1-yl)methyl)-3-(trifluoromethyl)phenyl)benzamide (70 mg, 0.11 mmol), methanamine hydrochloride (76 mg, 1.1 mmol), Pd₂(dba)₃ (11 mg, 0.011 mmol), X-Phos (11 mg, 0.022 mmol) and Cs₂CO₃ (430 mg, 1.32 mmol) in toluene (5.0 mL) was stirred at 80° C. overnight. The mixture was diluted with ethyl acetate (50 mL), washed with brine (50 mL×2), dried over anhydrous Na₂SO₄, filtered, concentrated and purified by preparative HPLC (C18 column, CH₃CN/H₂O, containing 0.05% NH₄HCO₃) to give Compound (I-24) as white solid (9.2 mg, yield 13.4%). LCMS (m/z): 613.0 [M+H]⁺; ¹H NMR (400 MHz, DMSO-d₆) δ (PPM): 10.70 (s, 1H), 8.37 (s, 1H), 8.16 (d, J=1.6 Hz, 1H), 7.97-7.95 (m, 1H), 7.78 (s, 1H), 7.72 (d, J=8.4 Hz, 1H), 7.54 (d, J=6.4 Hz, 1H), 7.43-7.40 (m, 2H), 6.71 (s, 1H), 6.57-6.56 (m, 1H), 4.01 (s, 3H), 3.64 (s, 2H), 3.15 (br, 2H), 2.96 (s, 2H), 2.87 (d, J=4.8 Hz, 2H), 2.56-2.53 (m, 2H), 2.16 (s, 3H).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 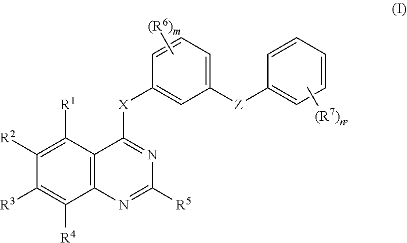
or a pharmaceutically acceptable salt, solvate, hydrate, polymorph, co-crystal, tautomer, stereoisomer, isotopically labeled compound, or prodrug thereof, wherein:
each instance of R¹, R², R³, R⁴, and R⁵ is independently hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —N(R^{d})₂, —OR^{a}, —SR^{a}, —CN, —SCN, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
provided that at least one of R¹, R², R³, and R⁴ is —N(R^{d})₂, —NR^{a}C(═O)R^{a}, —NR^{a}C(═O)OR^{a}, or —NR^{a}C(═O)N(R^{a})₂;
each instance of R^{a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on the same nitrogen atom are joined to form substituted or unsubstituted heterocyclyl or substituted or unsubstituted heteroaryl;
each instance of R^{d} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group; or two instances of R^{d} on the same nitrogen atom are joined to form substituted or unsubstituted heterocyclyl or substituted or unsubstituted heteroaryl; or one instance of R^{d} is joined with one of R¹, R², R³, R⁴, or the other instance of R^{d} to form an optionally substituted heterocyclyl or optionally substituted heteroaryl;
each instance of R⁶ and R⁷ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}—CN, —SCN, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, or —OC(═O)N(R^{b})₂;
each instance of R^{b} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{b} on the same nitrogen atom are joined to form substituted or unsubstituted heterocyclyl or substituted or unsubstituted heteroaryl;
m is 0, 1, 2, 3, or 4;
n is 0, 1, 2, 3, 4, or 5;
X is —N(R^{b})—, —O—, or —S—;
Z is —C(═O)N(R^{c})—, —N(R^{c})C(═O)—, —N(R^{c})C(═NR^{c})—, —C(═O)O—, or —C(═O)—; and
each instance of R^{c} is independently hydrogen, optionally substituted alkyl, optionally substituted acyl, or a nitrogen protecting group.

2. The compound of claim 1, or a pharmaceutically acceptable salt, solvate, hydrate, polymorph, co-crystal, tautomer, stereoisomer, isotopically labeled compound, or prodrug thereof, wherein X is —O—.

3. The compound of claim 1 or 2, or a pharmaceutically acceptable salt, solvate, hydrate, polymorph, co-crystal, tautomer, stereoisomer, isotopically labeled compound, or prodrug thereof, wherein R¹ is H.

4. The compound of any one of claims 1 to 3, or a pharmaceutically acceptable salt, solvate, hydrate, polymorph, co-crystal, tautomer, stereoisomer, isotopically labeled compound, or prodrug thereof, wherein R⁴ is H.

5. The compound of any one of claims 1 to 4, or a pharmaceutically acceptable salt, solvate, hydrate, polymorph, co-crystal, tautomer, stereoisomer, isotopically labeled compound, or prodrug thereof, wherein R⁵ is H.

6. The compound of any one of claims 1 to 5, of the Formula (II): 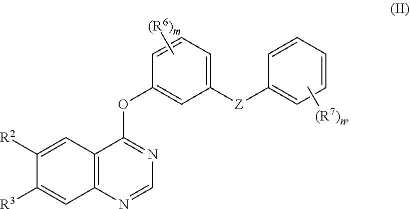
or a pharmaceutically acceptable salt, solvate, hydrate, polymorph, co-crystal, tautomer, stereoisomer, isotopically labeled compound, or prodrug thereof.

7. The compound of any one of claims 1 to 6, or a pharmaceutically acceptable salt, solvate, hydrate, polymorph, co-crystal, tautomer, stereoisomer, isotopically labeled compound, or prodrug thereof, wherein m is 1.

8. The compound of any one of claims 1 to 7, of the Formula (III): 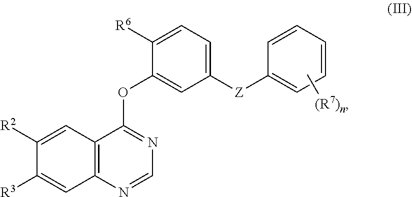
or a pharmaceutically acceptable salt, solvate, hydrate, polymorph, co-crystal, tautomer, stereoisomer, isotopically labeled compound, or prodrug thereof.

9. The compound of any one of claims 1 to 8, or a pharmaceutically acceptable salt, solvate, hydrate, polymorph, co-crystal, tautomer, stereoisomer, isotopically labeled compound, or prodrug thereof, wherein R⁶ is optionally substituted alkyl.

10. The compound of any one of claims 1 to 9, or a pharmaceutically acceptable salt, solvate, hydrate, polymorph, co-crystal, tautomer, stereoisomer, isotopically labeled compound, or prodrug thereof, wherein R⁶ is optionally substituted methyl.

11. The compound of any one of claims 1 to 10, or a pharmaceutically acceptable salt, solvate, hydrate, polymorph, co-crystal, tautomer, stereoisomer, isotopically labeled compound, or prodrug thereof, wherein R⁶ is unsubstituted methyl.

12. The compound of any one of claims 1 to 11, of the Formula (IV): 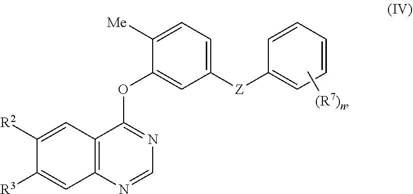
or a pharmaceutically acceptable salt, solvate, hydrate, polymorph, co-crystal, tautomer, stereoisomer, isotopically labeled compound, or prodrug thereof.

13. The compound of any one of claims 1 to 12, or a pharmaceutically acceptable salt, solvate, hydrate, polymorph, co-crystal, tautomer, stereoisomer, isotopically labeled compound, or prodrug thereof, wherein Z is —C(═O)N(R^{c})—.

14. The compound of any one of claims 1 to 13, or a pharmaceutically acceptable salt, solvate, hydrate, polymorph, co-crystal, tautomer, stereoisomer, isotopically labeled compound, or prodrug thereof, wherein R^{c} is H.

15. The compound of any one of claims 1 to 14, of the Formula (V): 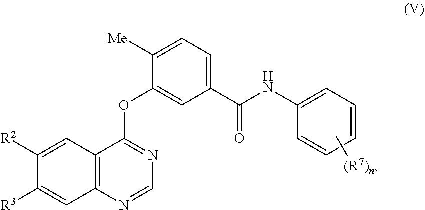
or a pharmaceutically acceptable salt, solvate, hydrate, polymorph, co-crystal, tautomer, stereoisomer, isotopically labeled compound, or prodrug thereof.

16. The compound of any one of claims 1 to 15, or a pharmaceutically acceptable salt, solvate, hydrate, polymorph, co-crystal, tautomer, stereoisomer, isotopically labeled compound, or prodrug thereof, wherein n is 1.

17. The compound of any one of claims 1 to 16, of the Formula (VI): 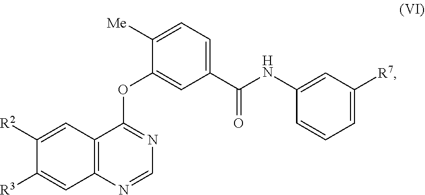
or a pharmaceutically acceptable salt, solvate, hydrate, polymorph, co-crystal, tautomer, stereoisomer, isotopically labeled compound, or prodrug thereof.

18. The compound of any one of claims 1 to 15, or a pharmaceutically acceptable salt, solvate, hydrate, polymorph, co-crystal, tautomer, stereoisomer, isotopically labeled compound, or prodrug thereof, wherein n is 2.

19. The compound of any one of claims 1 to 15 or 18, of the Formula (VII): 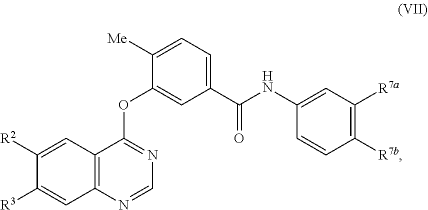
or a pharmaceutically acceptable salt, solvate, hydrate, polymorph, co-crystal, tautomer, stereoisomer, isotopically labeled compound, or prodrug thereof;
wherein each instance of R^{7a} or R^{7b} is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}—CN, —SCN, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, or —OC(═O)N(R^{b})₂.

20. The compound of any one of claims 1 to 19, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is optionally substituted alkyl.

21. The compound of any one of claims 1 to 20, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula: 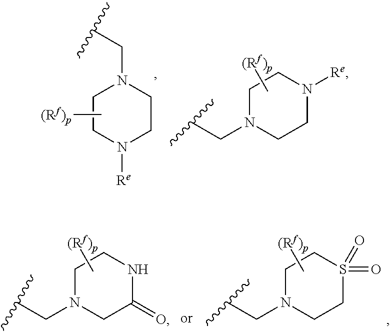
wherein R^{e} is hydrogen, substituted or unsubstituted alkyl, or substituted or unsubstituted acyl, or a nitrogen protecting group;
each instance of R^{f} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}—CN, —SCN, —C(═O)R^{g}, —C(═O)OR^{g}, —C(═O)N(R^{g})₂, —NO₂, —NR^{g}C(═O)R^{g}, —NR^{g}C(═O)OR^{g}, —NR^{g}C(═O)N(R^{g})₂, —OC(═O)R^{g}, —OC(═O)OR^{g}, or —OC(═O)N(R^{g})₂, wherein each instance of R^{g} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom; and
p is 0, 1, 2, 3, 4, 5, 6, 7, or 8.

22. The compound of claim 21, or a pharmaceutically acceptable salt, solvate, hydrate, polymorph, co-crystal, tautomer, stereoisomer, isotopically labeled compound, or prodrug thereof, wherein p is 0.

23. The compound of any one of claims 1 to 22, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula:

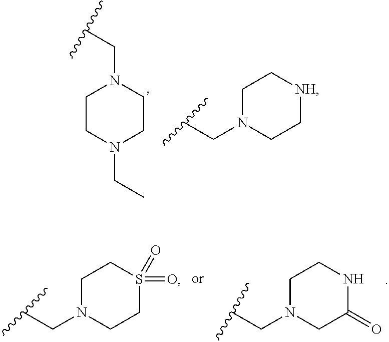

24. The compound of any one of claims 1 to 20, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is C₁₋₆ perhaloalkyl.

25. The compound of any one of claims 1 to 20 or 24, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is —CF₃.

26. The compound of any one of claims 1 to 20, 24, or 25, of the Formula (VI-a): 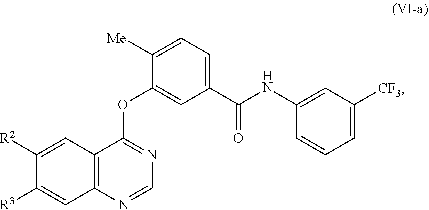
or a pharmaceutically acceptable salt, solvate, hydrate, polymorph, co-crystal, tautomer, stereoisomer, isotopically labeled compound, or prodrug thereof.

27. The compound of any one of claims 1 to 19 or 18 to 25, of the Formula (VII-a): 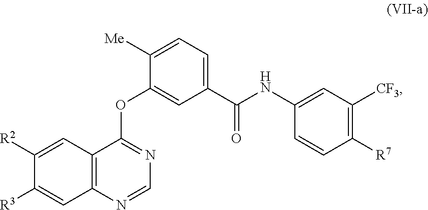
or a pharmaceutically acceptable salt, solvate, hydrate, polymorph, co-crystal, tautomer, stereoisomer, isotopically labeled compound, or prodrug thereof.

28. The compound of any one of claims 1 to 19 or 18 to 25, of the Formula (VII-b): 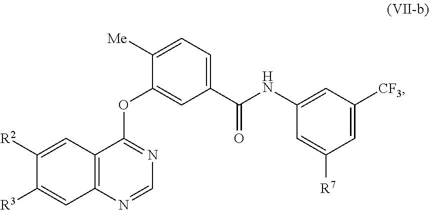
or a pharmaceutically acceptable salt, solvate, hydrate, polymorph, co-crystal, tautomer, stereoisomer, isotopically labeled compound, or prodrug thereof.

29. The compound of any one of claims 1 to 15, 18 to 25, or 27, of the Formula (VII-c): 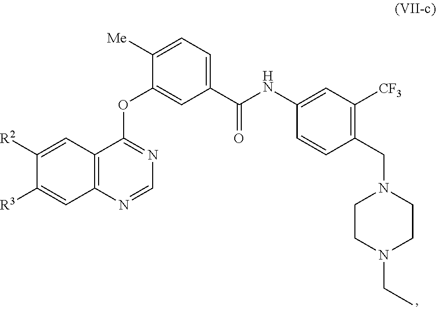
or a pharmaceutically acceptable salt, solvate, hydrate, polymorph, co-crystal, tautomer, stereoisomer, isotopically labeled compound, or prodrug thereof.

30. The compound of any one of claims 1 to 25, 27, or 28, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is optionally substituted acyl.

31. The compound of any one of claims 1 to 25, 27, 28, or 30, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula: 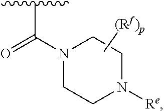
wherein R^{e} is hydrogen, substituted or unsubstituted alkyl, or substituted or unsubstituted acyl, or a nitrogen protecting group;
each instance of R^{f} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}—CN, —SCN, —C(═O)R^{g}, —C(═O)OR^{g}, —C(═O)N(R^{g})₂, —NO₂, —NR^{g}C(═O)R^{g}, —NR^{g}C(═O)OR^{g}, —NR^{g}C(═O)N(R^{g})₂, —OC(═O)R^{g}, —OC(═O)OR^{g}, or —OC(═O)N(R^{g})₂, wherein each instance of R^{g} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom; and
p is 0, 1, 2, 3, 4, 5, 6, 7, or 8.

32. The compound of claim 30, or a pharmaceutically acceptable salt, solvate, hydrate, polymorph, co-crystal, tautomer, stereoisomer, isotopically labeled compound, or prodrug thereof, wherein p is 0.

33. The compound of any one of claims 1 to 25, 27, 28, or 30, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula:

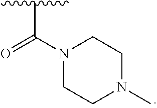 [SMILES: *C(=O)N1CCN(C)CC1]

34. The compound of any one of claims 1 to 25, 27, or 28, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is optionally substituted heterocyclyl.

35. The compound of any one of claims 1 to 25, 27, 28, or 34, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula: 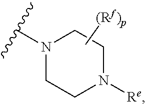
wherein R^{e} is hydrogen, substituted or unsubstituted alkyl, or substituted or unsubstituted acyl, or a nitrogen protecting group;
each instance of R^{f} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}—CN, —SCN, —C(═O)R^{g}, —C(═O)OR^{g}, —C(═O)N(R^{g})₂, —NO₂, —NR^{g}C(═O)R^{g}, —NR^{g}C(═O)OR^{g}, —NR^{g}C(═O)N(R^{g})₂, —OC(═O)R^{g}, —OC(═O)OR^{g}, or —OC(═O)N(R^{g})₂, wherein each instance of R^{g} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom; and
p is 0, 1, 2, 3, 4, 5, 6, 7, or 8.

36. The compound of claim 33, or a pharmaceutically acceptable salt, solvate, hydrate, polymorph, co-crystal, tautomer, stereoisomer, isotopically labeled compound, or prodrug thereof, wherein p is 0.

37. The compound of any one of claims 1 to 25, 27, 28, 34, or 35, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula:

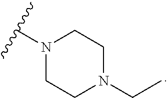 [SMILES: *N1CCN(CC)CC1]

38. The compound of any one of claims 1 to 25, 27, or 28, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is optionally substituted heteroaryl.

39. The compound of any one of claims 1 to 25, 27, 28, or 38, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula: 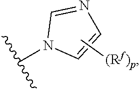
wherein each instance of R is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}—CN, —SCN, —C(═O)R^{g}, —C(═O)OR^{g}, —C(═O)N(R^{g})₂, —NO₂, —NR^{g}C(═O)R^{g}, —NR^{g}C(═O)OR^{g}, —NR^{g}C(═O)N(R^{g})₂, —OC(═O)R^{g}, —OC(═O)OR^{g}, or —OC(═O)N(R^{g})₂, wherein each instance of R^{g} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom; and
p is 0, 1, 2, or 3.

40. The compound of claim 36, or a pharmaceutically acceptable salt, solvate, hydrate, polymorph, co-crystal, tautomer, stereoisomer, isotopically labeled compound, or prodrug thereof, wherein p is 0.

41. The compound of any one of claims 1 to 25, 27, 28, 38, or 39, or a pharmaceutically acceptable salt, solvate, hydrate, polymorph, co-crystal, tautomer, stereoisomer, isotopically labeled compound, or prodrug thereof, wherein at least one instance of R⁷, R^{7a}, or R^{7b} is of the formula:

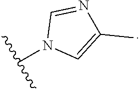 [SMILES: *n1cnc(C)c1]

42. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is —OR^{a}, —OC(═O)R^{a}, —OC(═O)OR^{a}, or —OC(═O)N(R^{a})₂.

43. The compound of any one of claims 1 to 42, or a pharmaceutically acceptable salt, solvate, hydrate, polymorph, co-crystal, tautomer, stereoisomer, isotopically labeled compound, or prodrug thereof, wherein R² is —OR^{a}.

44. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is —N(R^{d})₂, —NR^{a}C(═O)R^{a}, —NR^{a}C(═O)OR^{a}, or —NR^{a}C(═O)N(R^{a})₂.

45. The compound of any one of claims 1 to 44, or a pharmaceutically acceptable salt, solvate, hydrate, polymorph, co-crystal, tautomer, stereoisomer, isotopically labeled compound, or prodrug thereof, wherein R^{a} is substituted or unsubstituted alkyl.

46. The compound of any one of claims 1 to 45, or a pharmaceutically acceptable salt, solvate, hydrate, polymorph, co-crystal, tautomer, stereoisomer, isotopically labeled compound, or prodrug thereof, wherein R^{a} is optionally substituted C₁-C₆ alkyl.

47. The compound of any one of claims 1 to 46, or a pharmaceutically acceptable salt, solvate, hydrate, polymorph, co-crystal, tautomer, stereoisomer, isotopically labeled compound, or prodrug thereof, wherein R^{a} is optionally substituted methyl.

48. The compound of any one of claims 1 to 47, or a pharmaceutically acceptable salt, solvate, hydrate, polymorph, co-crystal, tautomer, stereoisomer, isotopically labeled compound, or prodrug thereof, wherein R^{a} is unsubstituted methyl.

49. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is H.

50. The compound of any one of claims 1 to 49, or a pharmaceutically acceptable salt, solvate, hydrate, polymorph, co-crystal, tautomer, stereoisomer, isotopically labeled compound, or prodrug thereof, wherein R³ is —N(R^{d})₂, —NR^{a}C(═O)R^{a}, —NR^{a}C(═O)OR^{a}, or —NR^{a}C(═O)N(R^{a})₂.

51. The compound of any one of claims 1 to 49, or a pharmaceutically acceptable salt, solvate, hydrate, polymorph, co-crystal, tautomer, stereoisomer, isotopically labeled compound, or prodrug thereof, wherein R³ is —OR^{a}, —OC(═O)R^{a}, —OC(═O)OR^{a}, or —OC(═O)N(R^{a})₂.

52. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is —N(R^{d})₂, —NR^{a}C(═O)R^{a}, —NR^{a}C(═O)OR^{a}, or —NR^{a}C(═O)N(R^{a})₂; and
R³ is —OR^{a}, —OC(═O)R^{a}, —OC(═O)OR^{a}, or —OC(═O)N(R^{a})₂.

53. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is —N(R^{d})₂, —NR^{a}C(═O)R^{a}, —NR^{a}C(═O)OR^{a}, or —NR^{a}C(═O)N(R^{a})₂; and
R³ is —OR^{a}.

54. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is —OR^{a}, —OC(═O)R^{a}, —OC(═O)OR^{a}, or —OC(═O)N(R^{a})₂; and
R³ is —N(R^{d})₂, —NR^{a}C(═O)R^{a}, —NR^{a}C(═O)OR^{a}, or —NR^{a}C(═O)N(R^{a})₂.

55. The compound of any one of claims 1 to 41, or a pharmaceutically acceptable salt, solvate, hydrate, polymorph, co-crystal, tautomer, stereoisomer, isotopically labeled compound, or prodrug thereof, wherein R² is —OR^{a}; and
R³ is —N(R^{d})₂, —NR^{a}C(═O)R^{a}, —NR^{a}C(═O)OR^{a}, or —NR^{a}C(═O)N(R^{a})₂.

56. The compound of any one of claims 1 to 55, or a pharmaceutically acceptable salt, solvate, hydrate, polymorph, co-crystal, tautomer, stereoisomer, isotopically labeled compound, or prodrug thereof, wherein R^{d} is substituted or unsubstituted alkyl.

57. The compound of any one of claims 1 to 56, or a pharmaceutically acceptable salt, solvate, hydrate, polymorph, co-crystal, tautomer, stereoisomer, isotopically labeled compound, or prodrug thereof, wherein R^{d} is optionally substituted C₁-C₆ alkyl.

58. The compound of any one of claims 1 to 57, or a pharmaceutically acceptable salt, solvate, hydrate, polymorph, co-crystal, tautomer, stereoisomer, isotopically labeled compound, or prodrug thereof, wherein R^{d} is optionally substituted methyl.

59. The compound of any one of claims 1 to 58, or a pharmaceutically acceptable salt, solvate, hydrate, polymorph, co-crystal, tautomer, stereoisomer, isotopically labeled compound, or prodrug thereof, wherein R^{d} is unsubstituted methyl.

60. The compound of any one of claims 1 to 48 or 50 to 59 of the formula: 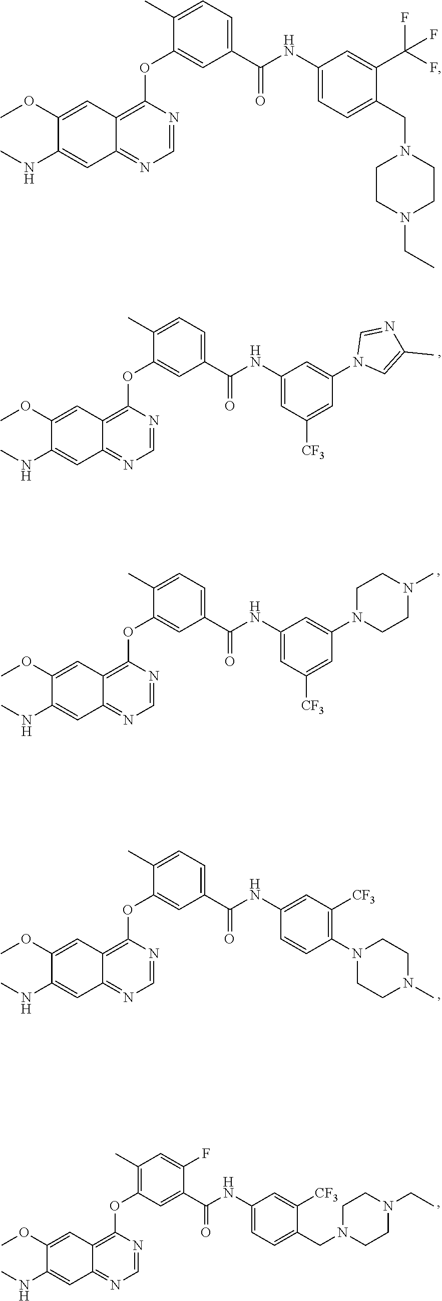 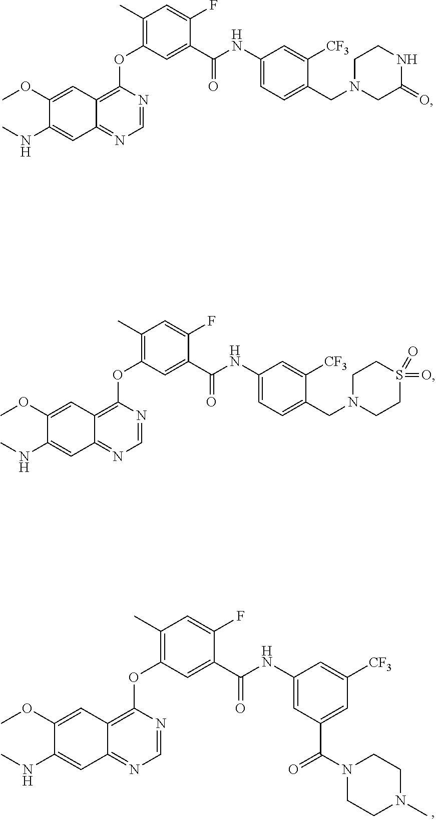 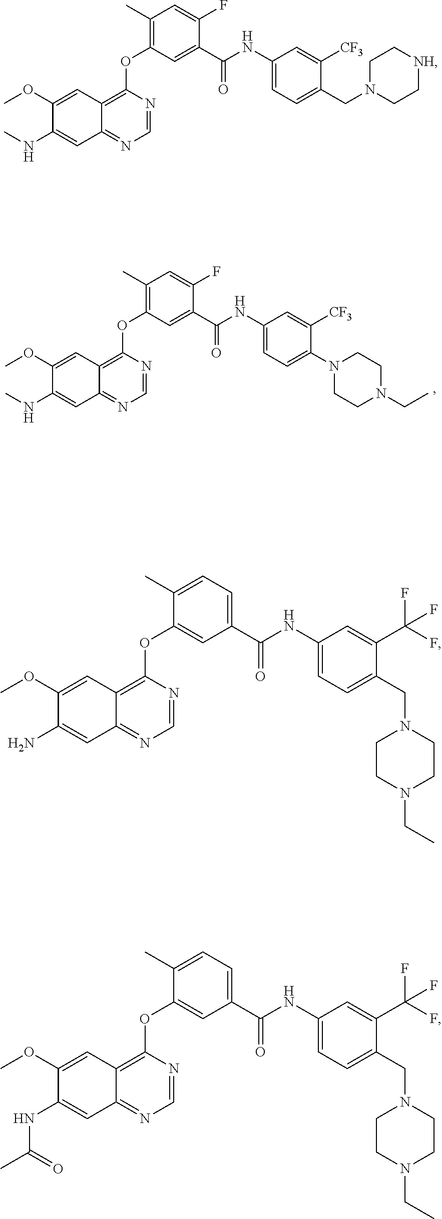
or a pharmaceutically acceptable salt, solvate, hydrate, polymorph, co-crystal, tautomer, stereoisomer, isotopically labeled compound, or prodrug thereof.

61. The compound of any one of claims 1 to 41 or 42 to 59, of the formula: 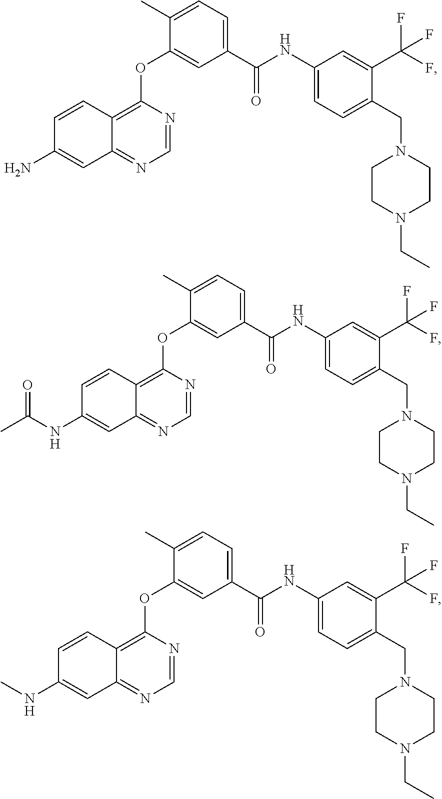
or a pharmaceutically acceptable salt, solvate, hydrate, polymorph, co-crystal, tautomer, stereoisomer, isotopically labeled compound, or prodrug thereof.

62. The compound of any one of claims 1 to 55, or a pharmaceutically acceptable salt, solvate, hydrate, polymorph, co-crystal, tautomer, stereoisomer, isotopically labeled compound, or prodrug thereof, wherein R^{d} is optionally substituted ethyl.

63. The compound of any one of claims 1 to 56 or 62, or a pharmaceutically acceptable salt, solvate, hydrate, polymorph, co-crystal, tautomer, stereoisomer, isotopically labeled compound, or prodrug thereof, wherein R^{d} is ethyl substituted with a nitrogen containing heterocyclic ring.

64. The compound of any one of claims 1 to 56, 62, or 63, or a pharmaceutically acceptable salt, solvate, hydrate, polymorph, co-crystal, tautomer, stereoisomer, isotopically labeled compound, or prodrug thereof, wherein R^{d} is of the formula: 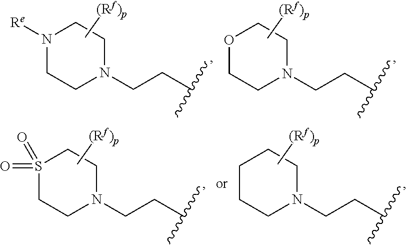
wherein R^{e} is hydrogen, substituted or unsubstituted alkyl, or substituted or unsubstituted acyl, or a nitrogen protecting group; and
each instance of R^{f} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}—CN, —SCN, —C(═O)R^{g}, —C(═O)OR^{g}, —C(═O)N(R^{g})₂, —NO₂, —NR^{g}C(═O)R^{g}, —NR^{g}C(═O)OR^{g}, —NR^{g}C(═O)N(R^{g})₂, —OC(═O)R^{g}, —OC(═O)OR^{g}, or —OC(═O)N(R^{g})₂, wherein each instance of R^{g} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom.

65. The compound of claim 64, or a pharmaceutically acceptable salt, solvate, hydrate, polymorph, co-crystal, tautomer, stereoisomer, isotopically labeled compound, or prodrug thereof, wherein p is 0.

66. The compound of any one of claims 1 to 56 or 62 to 65, or a pharmaceutically acceptable salt, solvate, hydrate, polymorph, co-crystal, tautomer, stereoisomer, isotopically labeled compound, or prodrug thereof, wherein R^{d} is of the formula:

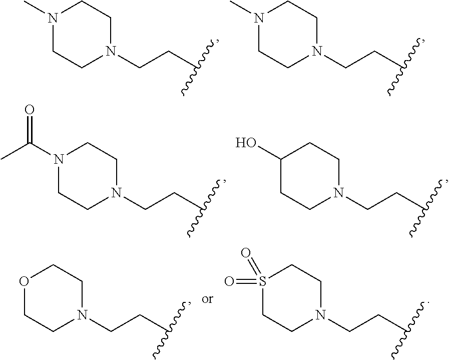

67. The compound of any one of claims 1 to 56 or 62 to 66, of the formula: 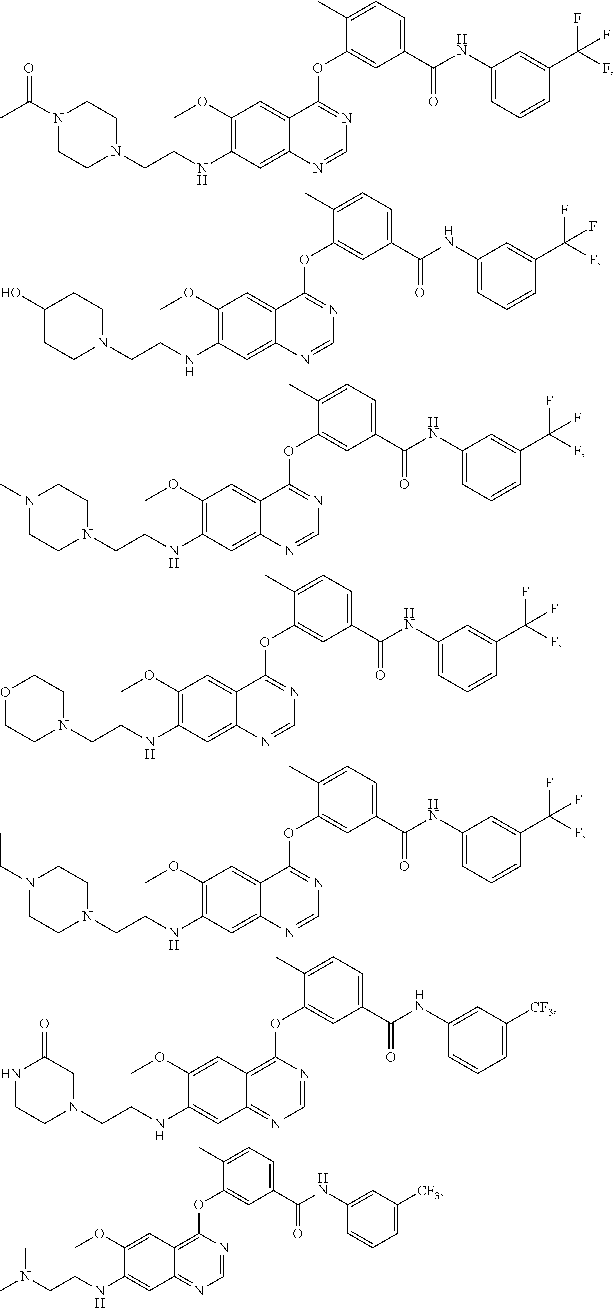 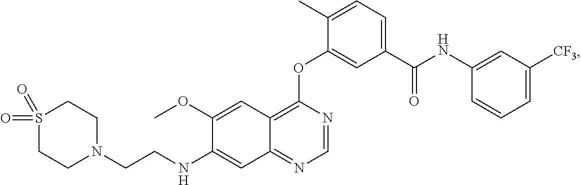 [SMILES: COc1cc2c(Oc3cc(C(=O)Nc4cccc(C(F)(F)F)c4)ccc3C)ncnc2cc1NCCN1CCS(=O)(=O)CC1]
or a pharmaceutically acceptable salt, solvate, hydrate, polymorph, co-crystal, tautomer, stereoisomer, isotopically labeled compound, or prodrug thereof.

68. The compound of any one of claims 1 to 41 or 50, or a pharmaceutically acceptable salt, solvate, hydrate, polymorph, co-crystal, tautomer, stereoisomer, isotopically labeled compound, or prodrug thereof, wherein one instance of R^{d} is joined with one instance of R², or R⁴ to form an optionally substituted heteroaryl ring.

69. The compound of any one of claims 1 to 41, 50, or 68, or a pharmaceutically acceptable salt, solvate, hydrate, polymorph, co-crystal, tautomer, stereoisomer, isotopically labeled compound, or prodrug thereof, wherein one instance of R^{d} is joined with one instance of R² to form an optionally substituted heteroaryl ring.

70. The compound of any one of claims 1 to 41, 50, 68, or 69, or a pharmaceutically acceptable salt, solvate, hydrate, polymorph, co-crystal, tautomer, stereoisomer, isotopically labeled compound, or prodrug thereof, wherein one instance of R^{d} is joined with one instance of R² to form an optionally substituted pyrrole, pyrazole, or imidazole.

71. The compound of any one of claims 1 to 41, 50, or 68 to 70, or a pharmaceutically acceptable salt, solvate, hydrate, polymorph, co-crystal, tautomer, stereoisomer, isotopically labeled compound, or prodrug thereof, of the Formulae (II-a), (II-b), or (II-c): 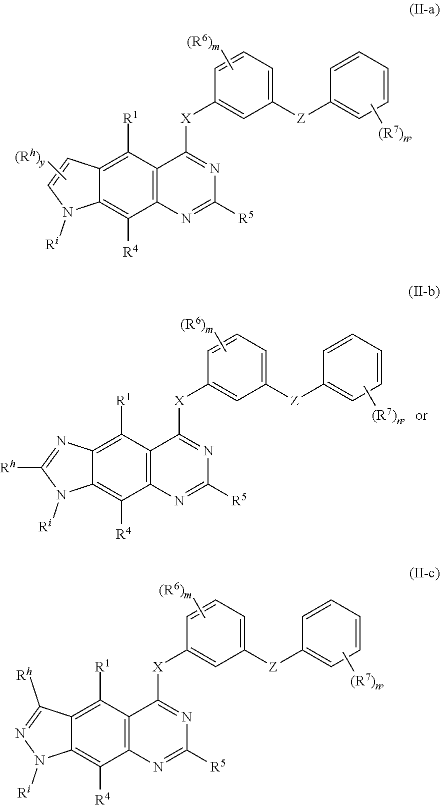
wherein Rⁱ is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl;
each instance of R^{h} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{j}, —N(R^{j})₂, —SR^{j}—CN, —SCN, —C(═O)R^{j}, —C(═O)OR^{j}, —C(═O)N(R^{j})₂, —NO₂, —NR^{j}C(═O)R^{j}, —NR^{j}C(═O)OR^{j}, —NR^{j}C(═O)N(R^{j})₂, —OC(═O)R^{j}, —OC(═O)OR^{j}, or —OC(═O)N(R^{j})₂, wherein each instance of R^{j} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom; and
y is 1 or 2.

72. The compound of claim 71, or a pharmaceutically acceptable salt, solvate, hydrate, polymorph, co-crystal, tautomer, stereoisomer, isotopically labeled compound, or prodrug thereof, wherein Rⁱ is optionally substituted C₁-C₆ alkyl.

73. The compound of claim 71 or 72, or a pharmaceutically acceptable salt, solvate, hydrate, polymorph, co-crystal, tautomer, stereoisomer, isotopically labeled compound, or prodrug thereof, wherein Rⁱ is optionally substituted methyl.

74. The compound of any one of claims 71 to 73, or a pharmaceutically acceptable salt, solvate, hydrate, polymorph, co-crystal, tautomer, stereoisomer, isotopically labeled compound, or prodrug thereof, wherein Rⁱ is unsubstituted methyl.

75. The compound of claim 71, or a pharmaceutically acceptable salt, solvate, hydrate, polymorph, co-crystal, tautomer, stereoisomer, isotopically labeled compound, or prodrug thereof, wherein Rⁱ is hydrogen.

76. The compound of any one of claims 71 to 75, or a pharmaceutically acceptable salt, solvate, hydrate, polymorph, co-crystal, tautomer, stereoisomer, isotopically labeled compound, or prodrug thereof, wherein at least one instance of R^{h} is hydrogen.

77. The compound of any one of claims 1 to 41, 50, or 68 to 76, of the formula: 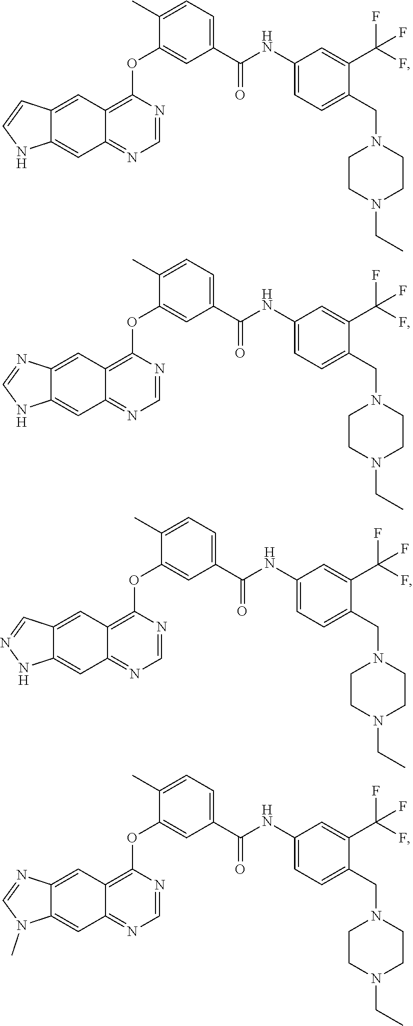
or a pharmaceutically acceptable salt, solvate, hydrate, polymorph, co-crystal, tautomer, stereoisomer, isotopically labeled compound, or prodrug thereof.

78. A pharmaceutical composition comprising a compound of any one of claims 1 to 77, or a pharmaceutically acceptable salt, solvate, hydrate, polymorph, co-crystal, tautomer, stereoisomer, isotopically labeled derivative, or prodrug thereof, and a pharmaceutically acceptable excipient.

79. The pharmaceutical composition of claim 78 further comprising an additional pharmaceutical agent.

80. The pharmaceutical composition of claim 79, wherein the additional pharmaceutical agent is an anti-cancer agent.

81. The pharmaceutical composition of claim 80, wherein the additional pharmaceutical agent is a chemotherapeutic agent.

82. The pharmaceutical composition of claim 81, wherein the additional pharmaceutical agent is a BCL-2 inhibitor.

83. The pharmaceutical composition of claim 82, wherein the additional pharmaceutical agent is venetoclax.

84. The pharmaceutical composition of claim 79 or 80, wherein the additional pharmaceutical agent is a proteasome inhibitor.

85. The pharmaceutical composition of claim 84, wherein the proteasome inhibitor is bortezomib, carfilzomib, ixazomib, or oprozomib.

86. The pharmaceutical composition of claim 79 or 80, wherein the additional pharmaceutical agent is a monoclonal antibody.

87. The pharmaceutical composition of claim 86, wherein the monoclonal antibody is rituximab, daratumumab, ofatumumab, or obinutuzumab.

88. The pharmaceutical composition of claim 79 or 80, wherein the additional pharmaceutical agent is a CXCR4 antagonist.

89. The pharmaceutical composition of claim 88, wherein the CXCR4 antagonist is KRH-3955, plerixafor, motixafortide, or a T140 analog.

90. A method of treating a disease in a subject in need thereof, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.

91. A method of treating a disease associated with an MYD88 mutation in a subject in need thereof, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.

92. A method of treating a proliferative disease in a subject in need thereof, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.

93. The method of any one of claims 90 to 92, wherein the disease is a proliferative disease and the proliferative disease is cancer.

94. The method of claim 93, wherein the cancer is breast cancer, colon cancer, stomach cancer, testicular cancer, or cancer of the central nervous system.

95. The method of claim 94, wherein the cancer is lymphoma.

96. The method of claim 95, wherein the lymphoma is a B-cell lymphoma.

97. The method of claim 96, wherein the B-cell lymphoma is lymphoplasmacytic lymphoma.

98. The method of claim 97, wherein the lymphoplasmacytic lymphoma is associated with immunoglobulin M (IgM) secreting lymphoplasmacytic lymphoma.

99. The method of claim 98, wherein the lymphoplasmacytic lymphoma is Waldenström's macroglobulinemia.

100. The method of claim 97, wherein the lymphoplasmacytic lymphoma is non-IgM secreting lymphoplasmacytic lymphoma.

101. The method of claim 96, wherein the B-cell lymphoma is diffuse large B-cell lymphoma (DLBCL).

102. The method of claim 101, wherein, the diffuse large B-cell lymphoma is activated B-cell-like (ABC)-DLBCL.

103. The method of claim 101, wherein, the diffuse large B-cell lymphoma is germinal center B-cell-like (GBC)-DLBCL).

104. The method of claim 96, wherein the B-cell lymphoma is follicular lymphoma.

105. The method of claim 96, wherein the B-cell lymphoma is marginal zone B-cell lymphoma.

106. The method of claim 96, wherein the B-cell lymphoma is small lymphocytic lymphoma.

107. The method of claim 106, wherein the small lymphocytic lymphoma is mantle cell lymphoma.

108. The method of claim 93, wherein the cancer is leukemia.

109. The method of claim 108, wherein the leukemia is myelogenous leukemia.

110. The method of claim 109, wherein the myelogenous leukemia is chronic myelogenous leukemia.

111. The method of claim 109, wherein the myelogenous leukemia is acute myelogenous leukemia.

112. The method of claim 111, wherein the acute myelogenous leukemia is mast cell leukemia.

113. The method of claim 108, wherein the leukemia is chronic lymphocytic leukemia.

114. The method of claim 93, wherein the cancer is myeloma.

115. The method of claim 114, wherein the myeloma is an IgM myeloma.

116. The method of claim 115, wherein the IgM myeloma is IgM multiple myeloma.

117. The method of claim 93, wherein the cancer is a myeloproliferative disease.

118. The method of claim 117, wherein the myeloproliferative disease is myelodysplastic syndrome.

119. The method of claim 93, wherein the proliferative disease is an IgM gammopathy.

120. The method of claim 119, wherein the IgM gammopathy is an IgM Monoclonal gammopathy of undetermined significance (MGUS).

121. The method of claim 119, wherein the IgM gammopathy is amyloid light chain (AL) amyloidosis.

122. The method of claim 92, wherein the proliferative disease is mastocytosis.

123. The method of claim 122, wherein the mastocytosis is systemic mastocytosis.

124. A method of treating a disease associated with aberrant activity of HCK in a subject in need thereof, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.

125. The method of claim 124, wherein the aberrant activity is overexpression.

126. A method of inhibiting the activity of a kinase in a subject in need thereof, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.

127. The method of any one of claims 74 to 126, wherein the disease is resistant to treatment with a BTK inhibitor.

128. The method of claim 127, wherein the BTK inhibitor is ibrutinib, CC-292, ONO-4059, evobrutinib, spebrutinib, BGB-3111, HM71224, or ACP-196.

129. The method of claim 128, wherein the BTK inhibitor is ibrutinib.

130. The method of any one of claims 72 to 129, wherein the subject is a mammal.

131. The method of any one of claims 72 to 129, wherein the subject is a human.

132. A method of inhibiting the activity of a kinase in a biological sample or cell, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.

133. The method of claim 132, wherein the kinase is an SRC family of cytoplasmic tyrosine kinases (SFKs).

134. The method of claim 132 or 133, wherein the kinase is a hematopoietic cell kinase (HCK).

135. The method of claim 132 or 133, wherein the kinase is LYN proto-oncogene tyrosine kinase (LYN).

136. The method of claim 132 or 133, wherein the kinase is a Tec family kinase.

137. The method of claim 132 or 136, wherein the kinase is Bruton's tyrosine kinase (BTK).

138. The method of claim 137, wherein the BTK is a BTK C481S mutant.

139. The method of claim 137 or 138, wherein the BTK is resistant to inhibition by Ibrutinib.

140. A kit comprising:
a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83; and
instructions for using the compound, or a pharmaceutically acceptable salt, solvate, hydrate, polymorph, co-crystal, tautomer, stereoisomer, isotopically labeled derivative, or prodrug thereof, or the pharmaceutical composition.

141. The kit of claim 140, wherein the instructions are for treating a disease selected from an IgM Monoclonal gammopathy of undetermined significance (MGUS), amyloid light chain (AL) amyloidosis), breast cancer, colon cancer, testicular cancer, CNS cancer, stomach cancer, IgM secreting lymphoplasmacytic lymphoma, non-IgM secreting lymphoplasmacytic lymphoma, activated B-cell-like (ABC)-DLBCL, germinal center B-cell-like (GBC)-DLBCL), follicular lymphoma, marginal zone B-cell lymphoma, small lymphocytic lymphoma, mantle cell lymphoma, IgM multiple myeloma, chronic lymphocytic leukemia (CLL), acute lymphoblastic leukemia, chronic myelogenous leukemia, acute myelogenous leukemia, and myelodysplastic syndrome.

142. The kit of claim 140, wherein the instructions are for a method of inhibiting the activity of a kinase in a biological sample or cell, the method comprising administering to the subject a therapeutically effective amount of a compound of any one of claims 1 to 77, or a pharmaceutically acceptable salt, solvate, hydrate, polymorph, co-crystal, tautomer, stereoisomer, isotopically labeled derivative, or prodrug thereof, or a pharmaceutical composition of any one of claims 78 to 83.
